SEQUENCE LISTING (114 sequences)

SEQ ID NO: 1 - protein, 668 aa, Chlamydomonas reinhardtii
MQCQLKHGARPQSQRPNWLPARAATLRPAVQHGVRRGLTLGVKAAAAPLEDKKMPADMTTRQYRRVVYDFALWAKHRDVNRYLYNLRTIPGSRIIRQLSQPMGVVLAWAALFGFYETCLEAGVLPSYLPKMTLMSAEPQGLTSFALSLLLVFRTNSSYGRFDEARKIWGGILNRARNIANQAVTFIPAEDQAGREAVGKWTVGFTRALQAHLQEDIDLRKELEKATPRWSKEEIDMLVNAQHRPIKAISVLSELTRQLSITQFQALQMQENCTFFYDALGGCERLLRTPIPVSYTRHTARFLTIWLAMLPLGLWERYHWSMLPVIALIGFLLLGIDEIGISIEEPFGILPLDAICGRAQTDVNSLLKEDPAVMKYVDDVRSGRVKSPPPLPPAPAAPAAAAAAAAAAARSVSPQPDVAKTLGSLFTNVRAGVGAVAPGAPLMPQAPVRSPSPTRSVSPSFPRASAGTGMPPPVGMNGATPRVAAAPPTPPPVSRPAAPAAAPAAGSGFTMPNFSASLSGLTGGAAAAAKSAADAASSKLTKMADSMSSGAAAPAPPAAPARPSTSPRPSASSPISSSADADRSDSSRRPVNWRDELQSLKATREPNGNGNGSGVAPAAGRADADEEALRRFGNLAGRSRSGNGGGGSSDTELSEANRPRTRPDWRNQL

SEQ ID NO: 2 - protein, 1690 aa, Chlamydomonas reinhardtii
MKATAGALSAAGTSSAAQLPAAAAARGSVRASPAAGQAKRWLLRPLQPGQPGSSSLLPVAALNGEGQGPAVGAADWSSFPFQLSDDPLLRRSQLLLAASRRLRGEEPFPTPLADAELDPSAPRTVAGNAASVPDSPAVVSPLPFTRVGGTRPALTTFQSAASPDAAAGASLGELAVAAARMSTSTASPAGLLAAATAAAAVPSLMAPAAASASPASAAAAAAATPGAAAWLAIDNLLSEAAYSLSQQLDNSGLGGRTLASKTAVWSSAGGSLPEGLDDLLYSLAAELDALGLTAAGQALAGAAKGAVAGLTGAAAELPRAAAQVYRSAADAASVATNLSASRNQGVTLITPSPLPPDAGGPDLTQLEPELLAAAGLTPNPAWDPFGTIRAAEALSRGEVVPEGLVVPPALVAKAAAAAPVVTGTPSVSGAATATAAATVEAATTAAAGTIVIPAPAPAPTAPVPAPPVVAAVPVAPAPAPVPPPAVAAAGAPPAPTVCPVPSVPEPSAVVPPPAVVPPAPAPPVVAAAPPSPLLPPAAPVVAEAPDLSSDKLNSAVQDLLAAGSPPPVDAAAAAANAAAPAAPAAAPLPADAEAALGQLSEALQRELKAVVGPDVDVEAAAADPSALAEAAGRAVDSALGSLDSGALEALGQLPPDVRLSSLLGAVLQSALDLVDAAVSGVRQADSEVVGGVAIVVVLGLAIRSLVSVLGNALGGPRGGAMPAASAGGGGVDAAGGAPRTLAEAVAAEGSARATGSRTSRALGVTALEAAALLNNEPKALLLDVRNSGDVYEQGLADLRPFRRGSGAASAALPYLDFRTTPTLANPSGLLGGSGGAGAAGSVVAAVDPQFVPRFKQLKGLGRDSRVLLLDSYGVEAPEAVALLRSDPDIERLLGGEGVSFVEGGFAGPEGWKLTGLPVMDPPEPAAEARGAAGAGRPLLRGGPLDTSGLVGGLAALQMRYPALLTRVLAVGAVGGVGVAAASRVDWGAVSRGGVALAALLLVADRALPTGVRPSAKLRSQLQAQLEAPADSNAAAAAASSQQPGDKRRAALILRALDLVDAVGDAVVKAGQTAFSAAGGAASAAAKTAAASATSAAATAWPTASAGMGSEAAGDAASARASTVMSNWRDVIDSGAEVSAAPATAASASPSITRSPSPGPAFAAGISRSLGDAFHSAVSAVKRAASPARQPVAAVSGSNSRSSSPTRSGQAADTRDLVAAVAAASAMNGTAVLPGMAPLTFPKASAGQVPGWGAEEEAAAAAVPDDWRQAAEAEAYAPAGTQLDADAAAAAALAAIDAALADNSAAAPAPVSFRSASRSSWRDEVAAEAVPVPAAPSTSRSRSVTNWRDQVEAEAARAATAAASADASAVNRQGDDNGRTGSSRRKQPLRTASPERAAAAAEAMRRLRSEAAGADDDGLRVGVMGGEDKFFGGDSGEWDEVQLERRRESLRAAAGADSADEEAEARGGRERELVTVGVSASRAAAREKEVGATAAAADPRAARGRSSSRRVVARTLSPERTSEVAAAMRRMRLEAGLPPNDGSGDHAAAGFASPSNGHRASVNGNGSANGNGSGASRYTPSVSPSASAVVPRDWRRELQSSAGEGAESSGVEGQAQPQRRAGSGRARVVVSAGSRAPSNWRQQVDGGSNGNGNGNGNGNGQSSPRHATPANLSPSERLAREARMRDWRARV

SEQ ID NO: 3 - protein, 19 aa, Artificial Sequence
  Synthetic Construct
RGTGDSPTRRAFGDWRKNL

SEQ ID NO: 4 - protein, 29 aa, Artificial Sequence
  Synthetic Construct
KAQPQQPGRSTSADWRRLVSGGDAAGKDA

SEQ ID NO: 5 - protein, 19 aa, Artificial Sequence
  Synthetic Construct
RNASPVRRTAIPANWRDAL

SEQ ID NO: 6 - protein, 29 aa, Artificial Sequence
  Synthetic Construct
SGADSRSPSPRTLAWREAAEAQEREQESK

SEQ ID NO: 7 - protein, 29 aa, Artificial Sequence
  Synthetic Construct
PPAPAPAPAKTKPDWREQAQAPVQAAAAA

SEQ ID NO: 8 - protein, 29 aa, Artificial Sequence
  Synthetic Construct
ASDSEGGETVTKANWREALAALHDAGSDG

SEQ ID NO: 9 - protein, 19 aa, Artificial Sequence
  Synthetic Construct
TELSEANRPRTRPDWRNQL

SEQ ID NO: 10 - protein, 29 aa, Artificial Sequence
  Synthetic Construct
ADADRSDSSRRPVNWRDELQSLKATREPN

SEQ ID NO: 11 - protein, 19 aa, Artificial Sequence
  Synthetic Construct
SPSERLAREARMRDWRARV

SEQ ID NO: 12 - protein, 29 aa, Artificial Sequence
  Synthetic Construct
ARVVVSAGSRAPSNWRQQVDGGSNGNGNG

SEQ ID NO: 13 - protein, 29 aa, Artificial Sequence
  Synthetic Construct
PSVSPSASAVVPRDWRRELQSSAGEGAES

SEQ ID NO: 14 - protein, 29 aa, Artificial Sequence
  Synthetic Construct
PAAPSTSRSRSVTNWRDQVEAEAARAATA

SEQ ID NO: 15 - protein, 29 aa, Artificial Sequence
  Synthetic Construct
APAPVSFRSASRSSWRDEVAAEAVPVPAA

SEQ ID NO: 16 - protein, 29 aa, Artificial Sequence
  Synthetic Construct
GAEEEAAAAAVPDDWRQAAEAEAYAPAGT

SEQ ID NO: 17 - protein, 29 aa, Artificial Sequence
  Synthetic Construct
GDAASARASTVMSNWRDVIDSGAEVSAAP

SEQ ID NO: 18 - protein, 19 aa, Artificial Sequence
  Synthetic Construct
KSKPEIKRTALPADWRKGL

SEQ ID NO: 19 - protein, 29 aa, Artificial Sequence
  Synthetic Construct
ASSAPAPARSSSASWRDAPASSSSSSADK

SEQ ID NO: 20 - protein, 29 aa, Artificial Sequence
  Synthetic Construct
KKAVTPSRSALPSNWKQELESLRSNSPAP

SEQ ID NO: 21 - protein, 29 aa, Artificial Sequence
  Synthetic Construct
KKAVTPSRSALPSNWKQELESLRSSSPAP

SEQ ID NO: 22 - protein, 29 aa, Artificial Sequence
  Synthetic Construct
ASSAPAPARSSSASWRDAAPASSAPARSS

SEQ ID NO: 23 - protein, 29 aa, Artificial Sequence
  Synthetic Construct
TNRVSPTRSVLPANWRQELESLRNGNGSS

SEQ ID NO: 24 - protein, 29 aa, Artificial Sequence
  Synthetic Construct
SGRVKTVKVAARGSWRESSTATVQASRAS

SEQ ID NO: 25 - protein, 19 aa, Artificial Sequence
  Synthetic Construct
RSATTGRSGSVPKDWRSSL

SEQ ID NO: 26 - protein, 29 aa, Artificial Sequence
  Synthetic Construct
RRDLKIKRTVLPANWRDSLDEDEPAKPAA

SEQ ID NO: 27 - protein, 14 aa, Artificial Sequence
  Synthetic Construct
  VARIANT 1: Xaa = Arg or Lys
  VARIANT 2, 3, 4, 5: Xaa = Any Amino Acid, and up to three can be present or absent
  VARIANT 7: Xaa = Any Amino Acid, and can be present or absent
  VARIANT 8: Xaa = Asp or Asn
  VARIANT 10: Xaa = Arg or Lys
  VARIANT 11, 12: Xaa = Any Amino Acid
  VARIANT 13: Xaa = Ala, Ile, Leu, or Val
  VARIANT 14: Xaa = Asp, Glu, or absent
XXXXXPXXWXXXXX

SEQ ID NO: 28 - protein, 6 aa, Artificial Sequence
  Synthetic Construct
  VARIANT 2: Xaa = Arg or Lys
  VARIANT 3, 4: Xaa = Any Amino Acid
  VARIANT 5: Xaa = Ala, Ile, Leu, or Val
  VARIANT 6: Xaa = Asp, Glu, or absent
WXXXXX

SEQ ID NO: 29 - protein, 130 aa, Chlamydomonas reinhardtii
MALVARPVLSARVAASRPRVAARKAVRVSAKYGENSRYFDLQDMENTTGSWDMYGVDEKKRYPDNQAKFFTQATDIISRRESLRALVALSGIAAIVTYGLKGAKDADLPITKGPQTTGENGKGGSVRSRL

SEQ ID NO: 30 - protein, 21 aa, Chlamydomonas reinhardtii
RESLRALVALSGIAAIVTYGL

SEQ ID NO: 31 - protein, 55 aa, Arabidopsis thaliana
MASSMLSSAAVVTSPAQATMVAPFTGLKSSASFPVTRKANNDITSITSNGGRVSC

SEQ ID NO: 32 - protein, 55 aa, Arabidopsis thaliana
MASSMFSSTAVVTSPAQATMVAPFTGLKSSASFPVTRKANNDITSITSNGGRVSC

SEQ ID NO: 33 - protein, 55 aa, Arabidopsis thaliana
MASSMLSSAAVVTSPAQATMVAPFTGLKSSAAFPVTRKTNKDITSIASNGGRVSC

SEQ ID NO: 34 - protein, 55 aa, Arabidopsis thaliana
MASSMLSSATMVASPAQATMVAPFNGLKSSAAFPATRKANNDITSITSNGGRVNC

SEQ ID NO: 35 - protein, 85 aa, Artificial Sequence
  Synthetic Construct
MAPSVMASSATTVAPFQGLKSTAGMPVARRSGNSSFGNVSNGGRIRCMQVWPIEGIKKFETLSYLPPLGNSSFGNVSNGGRIRCM

SEQ ID NO: 36 - protein, 705 aa, Volvox carteri f. nagariensis
MQSQLQPRLQLQGTRLNWLPQRSCVQRRSLRVDATSGAAPPPPAGKELSNDMVTRQYRRTVYDFSLWAKHRDVNRYLYNLKTIPGSRIIRTLGQPMGIVLAWAAMFGFYETCLESGVLPSYFPKLTLMSAEPQGLTSFALSLLLVFRTNSSYGRFDEARKIWGGILNRARNIANQAVTFIPAEDVAGREAVGKWAVGFCRALQAHLQEDANLREELQKAQPRWSREEIDMLCSAQHSWQQLQSCVNAFWPIKAISMLSELTRQLPISQFQALQMQENVTFFYDALGGCERLLRTPIPVSYTRILPLDAICTRAQTDVVSLLKDDPAVVKYISDVRQGRIAPPTEPPVAGAAPVAAAPPPPPPASAGGGISRSGSPTAQQQPDVMKTVTSMLHNVKAGIGAVAPAPPRPPSPQPRARSPRAASPGGPSPFPRASAGTGGAAAAVPSPPPIKPLTSSSSSSSGAVSKDSNNSTATAKKPASAPAASSAGFSMGFSGLADGAAAAAKSASAAAAKFSKIADSVVAGTPAAPASEAKRETAAAAAMQAQPRNTPSSSSSTPSAAPANGSSDDDRSSSGRRTAAAVNWREELAALRAGREDAEEPASASASYDREFPSSSWSFSSASSAAVVQSGDAEDEARRRFGGLAGRGARSDTTTSAAAVMRGNGNGLSENGYGNGYGNDNGNGNGNTVEARGARPRTRPDWRNQL

SEQ ID NO: 37 - protein, 1669 aa, Volvox carteri f. nagariensis
MAATSTATAASATASASPATASEPVGRAALAAALTTASIAAAAAVLPPAASASAVGGGAAGLTAAAASVASAATSGWLAIEELLEEAAYSLSQEVPDGPGRSPLEALARLASTSDGGSGSSIAAGAGLDLDLPQQLQGLLPPQVAESLAAGPALPVSLSGGLGSGPGGLGLASGGAAGSGSGGLLAAVPGGDRLSEVLEDLLYSLSQEINPAAAQDAVLSAAMAAAAGLAGAAEELPRAAASVYHSAAEAAAAAAAARAASAARVPSGGGSGGTALVVPDESQTQLAMATGVEYGAGEEYDMELDGVDDMTVAAGNLAWDPYDSVRAAEGALDAMVPDASAADAAVAAASSAVVDATAAVLPPIITASAAPSVADAATATALAVVEAATTAAAGTITINPPSVATLPSASAEPAAAAAAATAVSAAMAGEGAGADGQMGVYGMDPLDTQQLDELTTALQATLQSSLDMVEAALSGIRSADSTLVGGVTIAAVLAVVIRSLVSMAGAALSRTRGPGGGGGEAGGGGGGSAAAAARMRPVGVTALEAAAALNNDPQALLLDIRNGSDVQEQGLPDLRPFRRGAGATTVPLPYCDFRTTPTLANPSGSLLAAAAATGPAAGSPRGGKAAAAAALAPVAGPVTVSVDPLFCSKFKQLEGLNRDSRVFLMDSYGVEAPEAVLLLRSDLEVEGLLGAQGVKFVEGGFAGPEGWKPDLNPLTLLSSFLIFAAIQYYTADISSCHVYLPFMYPSLFGRTLAVGAVGGAGVAAASSLDWASVSRGAVGLAAAMLLTDRVLPPGVRPSGKIRQYLQSQLSDPQPNDGGSGASAAADLAAARRRTALLLRALDLAEAVGDAVVRAGGAAVAAAGSAARGAIAASGGGSAAAAAATAANAGPADVEAELETPPPPAAAAATIVLPHGMDSRQWSEAAEMAVAQVQQQQPAAPPPPENQTRSASPLQPKWRLGPSAPSAGSSSNSVDAVPASTAAAFSNSPPPPPPPVPRAPSPSVVGRLNDALRSAADAVVRAASPSSKSSAAAASRAGSPGGPSGGGSSSRSQPDTLELMTALATASALSNVDNGLLPGWPALNFPKASAGQMPQQPQLQPVPKAGDSEQEEEEEKDRGEMAPAATGRRGPIAKERQPAAAAAEQDNVPLFSSLSALSASYNAAEATALADMWSNWRQDLEAVAPPPPALPSSEDYDDEDDEGEVKGRRDSGSGDSPSGSRFSENDGRGRLYGSGDTNAVAASAAMPHMDLAATQRFGIPTHDRPASAGASTTAVGASSTAPLAGAAWKSATNSGADGSGAVARQQQQQQQRKPLRTSSPERAAAGGAALRRLRMRMDARDDDGLASGAYGSGSFFGGSSGDEGSNGDGGDGPAAAATAAGKRYGSTSAAVPASGTAWTEAWGRSAGAVDGGGGRGLPSCSPHSVTRRKQSLRTASPQRAAAAAAAMRQLRAEMGLPPNDGTDAADAVFARDWRRELDAAAAAAADTEPSGSASESEWEAAAAAATEPGDRTSLYGSVGGTASNGRTASGSIRSRNAAAVGATAAFPRSPSNWRVQVEGLDRPDSSTSSSSSRGFGGVPADWRTRIESGAPATATAIAADGLNGQDSSGSAAASIGSVYDDDVSTSGNNRYSRGSPYPSSPGGSRGSSRKLGAAERAARTARLQDWRARV

SEQ ID NO: 38 - protein, 211 aa, Volvox carteri f. nagariensis
MAAMVMKSSVATAVVRPARSSVRPCAVLKPAVKAATVTAPAQANKMMVWTPVNNKASMYHTDLLHLPCYNTKNPCFFQSGRGFRNPHGIRFLTARWLRWFAACKRPPGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 39 - protein, 185 aa, Volvox carteri f. nagariensis
MAAVIAKSSVATAVARPARSGVRPVAVLKPSVRATPVATPTQANKMMVWTPVNNKMFETFSYLPPLSDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 40 - protein, 284 aa, Volvox carteri f. nagariensis
MQLGGWGEFRRVLDGASLRVPVSLILHGPLRCRFDLEQEGFRVRDETLAKALEKLGRAPYHGQETPPYVDAAVWRWSCLWIAVKVITLDSLVRISYFLRVPGVYVVLGLTTQTANGSSRSVRPCAVLKPAVKAATVAAPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 41 - protein, 186 aa, Volvox carteri f. nagariensis
MAAIVAKSSVASAVARPSRNSVQRSVAALKPAVKAAPVTAPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 42 - protein, 185 aa, Volvox carteri f. nagariensis
MAALLAKSSVAAAVARPQRSSVRPCAALKPAVKAAPVATPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 43 - protein, 185 aa, Volvox carteri f. nagariensis
MAAIVAKSSVAAVVARPARSSVRPVAGLKPAVKAAPVAAPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 44 - protein, 185 aa, Volvox carteri f. nagariensis
MAAIVAKSSVATAVVRPARSSVRPVAVLKPAIKAAPVASPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 45 - protein, 20 aa, Artificial Sequence
  Synthetic Construct
  VARIANT 2, 4: Xaa = Ser or Thr
  VARIANT 7: Xaa = Ala or Val
  VARIANT 10: Xaa = Ala or Ser
  VARIANT 13: Xaa = Arg or Lys
VXPXRSXLPXNWXQELESLR

SEQ ID NO: 46 - protein, 21 aa, Artificial Sequence
  Synthetic Construct
PARSSSASWRDAAPASSAPAR

SEQ ID NO: 47 - protein, 21 aa, Artificial Sequence
  Synthetic Construct
  VARIANT 1: Xaa = Val or Pro
  VARIANT 3: Xaa = Pro or Ile
  VARIANT 4: Xaa = Gly, Ser, Thr, Tyr, Cys, Gln, or Asn
  VARIANT 6: Xaa = Ser or Thr
  VARIANT 7: Xaa = Ala or Val
  VARIANT 10: Xaa = Ser or Ala
  VARIANT 11: Xaa = Asn or Asp
  VARIANT 13: Xaa = Lys or Arg
  VARIANT 14: Xaa = Gln or Lys
  VARIANT 15: Xaa = Glu or Gly
  VARIANT 21: Xaa = Gly, Ser, Thr, Tyr, Cys, Gln, or Asn
XTXXRXXLPXXWXXXLESLRX

SEQ ID NO: 48 - protein, 25 aa, Artificial Sequence
  Synthetic Construct
  VARIANT 1: Xaa = Pro or Val
  VARIANT 2: Xaa = Ala or Lys
  VARIANT 3: Xaa = Arg or Val
  VARIANT 4, 5: Xaa = Ser or Ala
  VARIANT 6: Xaa = Ser or Arg
  VARIANT 7: Xaa = Ala or Gly
  VARIANT 11: Xaa = Asp or Glu
  VARIANT 12: Xaa = Ala or Ser
  VARIANT 13: Xaa = Ala, Val, Leu, Ile, Pro, Trp, Phe, or Met
  VARIANT 14: Xaa = Pro or Thr
  VARIANT 16: Xaa = Ser or Thr
  VARIANT 17: Xaa = Ser or Val
  VARIANT 19, 20: Xaa = Ala, Val, Leu, Ile, Pro, Trp, Phe, or Met
  VARIANT 22, 25: Xaa = Ser or Ala
XXXXXXXSWRXXXXAXXAXXRXSSX

SEQ ID NO: 49 - protein, 13 aa, Artificial Sequence
  Synthetic Construct
AVIYDVQAAIQED

SEQ ID NO: 50 - protein, 14 aa, Artificial Sequence
  Synthetic Construct
PMQVLREIVACTKA

SEQ ID NO: 51 - protein, 24 aa, Artificial Sequence
  Synthetic Construct
TNRVSPTRSVLPANWRQELESLRN

SEQ ID NO: 52 - protein, 317 aa, Chlamydomonas reinhardtii
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 53 - protein, 14 aa, Artificial Sequence
  Synthetic Construct
KVAARGSWRESSTA

SEQ ID NO: 54 - protein, 16 aa, Artificial Sequence
  Synthetic Construct
RSVLPANWRQELESLR

SEQ ID NO: 55 - protein, 12 aa, Artificial Sequence
  Synthetic Construct
PARSSSASWRDA

SEQ ID NO: 56 - protein, 16 aa, Artificial Sequence
  Synthetic Construct
RSALPSNWKQELESLR

SEQ ID NO: 57 - protein, 10 aa, Artificial Sequence
  Synthetic Construct
PARSSSASWR

SEQ ID NO: 58 - protein, 14 aa, Artificial Sequence
  Synthetic Construct
IKRTALPADWRKGL

SEQ ID NO: 59 - protein, 18 aa, Artificial Sequence
  Synthetic Construct
  VARIANT 2: Xaa = Ser or Thr
  VARIANT 5: Xaa = Val or Ala
  VARIANT 8: Xaa = Ser or Ala
  VARIANT 11: Xaa = Arg or Lys
PXRSXLPXNWXQELESLR

SEQ ID NO: 60 - protein, 140 aa, Chlamydomonas reinhardtii
MMVWTPVNNKMFETFSYLPPLTDEQIAAQVDYIVANGWIPCLEFAEADKAYVSNESAIRFGSVSCLYYDNRYWTMWKLPMFGCRDPMQVLREIVACTKAFPDAYVRLVAFDNQKQVQIMGFLVQRPKTARDFQPANKRSV

SEQ ID NO: 61 - protein, 140 aa, Chlamydomonas reinhardtii
MMVWTPVNNKMFETFSYLPPLSDEQIAAQVDYIVANGWIPCLEFAESDKAYVSNESAIRFGSVSCLYYDNRYWTMWKLPMFGCRDPMQVLREIVACTKAFPDAYVRLVAFDNQKQVQIMGFLVQRPKSARDWQPANKRSV

SEQ ID NO: 62 - protein, 11 aa, Chlamydomonas reinhardtii
KTVKVAARGSW

SEQ ID NO: 63 - protein, 13 aa, Chlamydomonas reinhardtii
RSVLPANWRQELE

SEQ ID NO: 64 - protein, 11 aa, Chlamydomonas reinhardtii
RSSSASWRDAA

SEQ ID NO: 65 - protein, 13 aa, Chlamydomonas reinhardtii
RSALPSNWKQELE

SEQ ID NO: 66 - protein, 8 aa, Chlamydomonas reinhardtii
RSSSASWR

SEQ ID NO: 67 - protein, 12 aa, Chlamydomonas reinhardtii
RTALPADWRKGL

SEQ ID NO: 68 - protein, 439 aa, Chlamydomonas reinhardtii
MQSSMRARVAGGARRAVGTAGRRLTVKVMNSNVLIANTKGGGHAFIGLYLAKELLKKGHKVTIMNDGDSDKLTKKNPYAKYSDLERQGLNVVWADPAKPSTYPRGTFDVVYDNNGKDLASCQPLIDHFKHKVDHYVFVSSAGAYKADPIEPMHVEGDARKSTAGHVEVEAYLEKARLPYTVFQPLYIYGPNTAKDCEQWFVDRIIRDRPVLLPAPGVQLTSLTHVEDVASMLAAVPGNRAAIGQHYNVCSDRCITFTGIAKAIGKALGKDPEIILYSPEKVGTGKSGKAEGFPFRTVHFFASADKAKRELGWKPKHDFQKDVQGLVNDYKANGRDKKEVDFSVDDKILAALGKSVPKSSSNSSVSASFSRLSSSGPKAEELPRSRSSFSPRRDLKIKRTVLPANWRDSLDEDEPAKPAAGRSATTGRSGSVPKDWRSSL

SEQ ID NO: 69 - protein, 13 aa, Chlamydomonas reinhardtii
RTVLPANWRDSLD

SEQ ID NO: 70 - protein, 6 aa, Chlamydomonas reinhardtii
PKDWRS

SEQ ID NO: 71 - protein, 13 aa, Chlamydomonas reinhardtii
RRPVNWRDELQSL

SEQ ID NO: 72 - protein, 12 aa, Chlamydomonas reinhardtii
RPRTRPDWRNQL

SEQ ID NO: 73 - protein, 7 aa, Chlamydomonas reinhardtii
NWRDVID

SEQ ID NO: 74 - protein, 9 aa, Chlamydomonas reinhardtii
PDDWRQAAE

SEQ ID NO: 75 - protein, 12 aa, Chlamydomonas reinhardtii
RSASRSSWRDEV

SEQ ID NO: 76 - protein, 13 aa, Chlamydomonas reinhardtii
RSRSVTNWRDQVE

SEQ ID NO: 77 - protein, 8 aa, Chlamydomonas reinhardtii
PRDWRREL

SEQ ID NO: 78 - protein, 31 aa, Chlamydomonas reinhardtii
RAPSNWRQQVDGGSNGNGNGNGNGNGQSSPR

SEQ ID NO: 79 - protein, 12 aa, Chlamydomonas reinhardtii
REARMRDWRARV

SEQ ID NO: 80 - protein, 11 aa, Chlamydomonas reinhardtii
RSTSADWRRLV

SEQ ID NO: 81 - protein, 11 aa, Chlamydomonas reinhardtii
RRAFGDWRKNL

SEQ ID NO: 82 - protein, 6 aa, Chlamydomonas reinhardtii
NWREAL

SEQ ID NO: 83 - protein, 10 aa, Chlamydomonas reinhardtii
KTKPDWREQA

SEQ ID NO: 84 - protein, 6 aa, Chlamydomonas reinhardtii
WREAAE

SEQ ID NO: 85 - protein, 12 aa, Chlamydomonas reinhardtii
RTAIPANWRDAL

SEQ ID NO: 86 - protein, 8 aa, Artificial Sequence
  Synthetic Construct
NWRQELES

SEQ ID NO: 87 - DNA, 5 bp, Artificial Sequence
  Synthetic Construct
ggggs

SEQ ID NO: 88 - DNA, 10 bp, Artificial Sequence
  Synthetic Construct
ggggsggggs

SEQ ID NO: 89 - DNA, 643 bp, Artificial Sequence
  Synthetic Construct
ctggagtacaactacaacagccacaacgtgtacatcaccgccgacaagcagaagaacggcatcaaggccaacttcaagatccgccacaacatcgaggacggcggcgtgcagctggccgaccactaccagcagaacacccccatcggcgacggccccgtgctgctgcccgacaaccactacctgagctaccagagcgccctgagcaaggaccccaacgagaagcgcgaccacatggtgctgctggagttcgtgaccgccgccggcatcaccctgggcatggacgagctgatcaagggcggtggcgactacaaggaccatgacggtgactataaggatcacgacatcgactacaaggacgatgacgacaagggcggcggcggctcgggcggcggcggcagccccgtgcgccgcaccgctatcccggccaactggcgtgacgcactgggcggcggcggctcgggcggcggcggctcgggcggcggcggcagccccgtgcgccgcaccgctatcccggccaactggcgtgacgcactgggcggcggcggctcgggcggcggcggctcgggcggcggcggcagccccgtgcgccgcaccgctatcccggccaactggcgtgacgcactgtaagatctctaactgcacg

SEQ ID NO: 90 - protein, 18 aa, Artificial Sequence
  Synthetic Construct
TRSVLPANWRQELESLRN

SEQ ID NO: 91 - protein, 18 aa, Artificial Sequence
  Synthetic Construct
TASVLPANWRQELESLRN

SEQ ID NO: 92 - protein, 18 aa, Artificial Sequence
  Synthetic Construct
TRSVLPAAWRQELESLRN

SEQ ID NO: 93 - protein, 18 aa, Artificial Sequence
  Synthetic Construct
TRSVLPANARQELESLRN

SEQ ID NO: 94 - protein, 18 aa, Artificial Sequence
  Synthetic Construct
TRSVLPANWAQELESLRN

SEQ ID NO: 95 - protein, 18 aa, Artificial Sequence
  Synthetic Construct
TRSVLPANWRAELESLRN

SEQ ID NO: 96 - protein, 18 aa, Artificial Sequence
  Synthetic Construct
TRSVLPANWRQALESLRN

SEQ ID NO: 97 - protein, 18 aa, Artificial Sequence
  Synthetic Construct
TRSVLPANWRQEAESLRN

SEQ ID NO: 98 - protein, 18 aa, Artificial Sequence
  Synthetic Construct
TRSVLPANWRQELASLRN

SEQ ID NO: 99 - protein, 18 aa, Artificial Sequence
  Synthetic Construct
TRSVLPANWRQELESARN

SEQ ID NO: 100 - protein, 18 aa, Artificial Sequence
  Synthetic Construct
TRSVLPANWRQELESLAN

SEQ ID NO: 101 - protein, 16 aa, Artificial Sequence
  Synthetic Construct
TRSVLPANWWRQELES

SEQ ID NO: 102 - protein, 12 aa, Artificial Sequence
  Synthetic Construct
TRSVLPANWRQE

SEQ ID NO: 103 - protein, 9 aa, Artificial Sequence
  Synthetic Construct
TRSVLPANW

SEQ ID NO: 104 - protein, 9 aa, Artificial Sequence
  Synthetic Construct
RQELESLRN

SEQ ID NO: 105 - protein, 12 aa, Artificial Sequence
  Synthetic Construct
ANWRQELESLRN

SEQ ID NO: 106 - protein, 15 aa, Artificial Sequence
  Synthetic Construct
VLPANWRQELESLRN

SEQ ID NO: 107 - protein, 200 aa, Tetrabaena socialis
MATLSSMRIGAAPRVAVARTQRASTVKVVAKGSWRDAPTVTAQPGRAASSAKPTSPTRSVLPANWRQELESLRGGNGNGAAAAPAAAAPRAQSAGWRDAPASAPAASAPMKKTATPARTALPANWKQELESLRSSSTGGASAAPAAAPARASSASWRDAPAAAPASKSSSPAPAGTNPWTGKSKIEIKRTALPADWRKGL

SEQ ID NO: 108 - protein, 245 aa, Gonium pectorale
MALSAMRVGAAPRAAVSRPQTVQVVARGSWRESSTVTATPAGRSSSAANRVSPTRSVLPANWRQELESLRNGNGNGSSAAAAPAPAPARSASASWRDAPAAAAPARPSSSPKKAVTPSRSSLPANWKQELEALRGGSSSSSASWRTESAPAAAPARSGSKKAVTPSRSSLPANWKQELESMRSASPAPSSAPAAPARSSSASWRSESGSSSSSAAADKAGTNPWTGKAKVEIKRTALPADWRKGL

SEQ ID NO: 109 - protein, 298 aa, Volvox carteri f. nagariensis
MAMSTMRVGAAPRVAVARSQSVKVVARGSWRESATVTAQPAGRASSSNRVSPTRSVLPANWRQELESLRNGNGNGAAAAPAPAPAPARSSSASWRSESSAAPAAASTPSRSTKKPVTPTRTSLPANWKQELESLRGSSSSSPAAAAPAPARSSSSPKKAVTPTRSSLPANWKQELESLRGGSSSAASAPAAAAAPAAASAPSRSPKKAVTPTRSSLPANWKQELESLRGGSSSSSSAPAPAAAPAPARSSSASWRTESPAPANESSSAAAKAGTNPWTGKAKIEIKRTTLPADWRRQL

SEQ ID NO: 110 - protein, 18 aa, Artificial Sequence
  Synthetic Construct
TRSVLPANWRQELESLRG

SEQ ID NO: 111 - protein, 13 aa, Artificial Sequence
  Synthetic Construct
DEQIAAQVDYIVA

SEQ ID NO: 112 - protein, 14 aa, Artificial Sequence
  Synthetic Construct
PMQVLREIVSCTRA

SEQ ID NO: 113 - protein, 11 aa, Artificial Sequence
  Synthetic Construct
NWRQELESLRN

SEQ ID NO: 114 - protein, 18 aa, Artificial Sequence
  Synthetic Construct
PARSSSASWRDAPASSSS

(19) United States
(12) Patent Application Publication
(10) Pub. No.: US 2022/0275390 A1
(43) Pub. Date: Sep. 1, 2022

(54) RUBISCO-BINDING PROTEIN MOTIFS AND USES THEREOF

(71) Applicants: The Trustees of Princeton University, Princeton, NJ (US); The Board of Trustees of the Leland Stanford Junior University, Stanford, CA (US); University of York, Heslington, York (GB); Howard Hughes Medical Institute, Chevy Chase, MD (US)

(72) Inventors: Martin C. Jonikas, Princeton, NJ (US); Moritz Meyer, Esch-sur-Alzette (LU); Shan He, Princeton, NJ (US); Alan Itakura, Stanford, CA (US); Vivian Chen Wong, San Jose, CA (US); Luke Colin Martin Mackinder, Norton, Malton, North Yorkshire (GB); Zhiheng Yu, Ashburn, VA (US); Doreen Matthies, Sterling, VA (US); Hui-Ting Chou, San Mateo, CA (US)

(73) Assignees: The Trustees of Princeton University, Princeton, NJ (US); The Board of Trustees of the Leland Stanford Junior University, Stanford, CA (US); University of York, Heslington, York (GB); Howard Hughes Medical Institute, Chevy Chase, MD (US)

(21) Appl. No.: 17/631,427

(22) PCT Filed: Jul. 30, 2020
(86) PCT No.: PCT/US2020/044326

§ 371 Date: Jan. 28, 2022

Related U.S. Application Data
(60) Provisional application No. 62/882,306, filed on Aug. 2, 2019.

(51) Int. Cl. C12N 15/82; C07K 14/405

(52) U.S. Cl. CPC C12N 15/8269; C07K 14/405; C07K 2319/08; C07K 2319/03

(57) ABSTRACT

Described herein are chimeric polypeptides that include one or more Rubisco-binding motifs (RBMs) and a heterologous polypeptide. Additional aspects of the present disclosure provide genetically altered plants having a chimeric polypeptide including one or more Rubisco-binding motifs (RBMs) and a heterologous polypeptide. Further aspects of the present disclosure relate to genetically altered plants having a stabilized polypeptide including two or more RBMs and one or both of an algal Rubisco-binding membrane protein (RBMP) and a Rubisco small subunit (SSU) protein. Other aspects of the present disclosure relate to methods of making such chimeric polypeptides and plants, as well as cultivating these genetically altered plants.

### CROSS-REFERENCE TO RELATED APPLICATIONS

This is the U.S. National Phase of International Application No PCT/US2020/044326, filed Jul. 30, 2020, which claims priority to and the benefit of the earlier filing date of U.S. Provisional Application No. 62/882,306, filed Aug. 2, 2019, which is hereby incorporated by reference in its entirety.

### STATEMENT REGARDING FEDERALLY SPONSORED RESEARCH OR DEVELOPMENT

This invention was made with government support under Grant Nos. 10S-1737710 and MCB-1935444 awarded by the National Science Foundation. The government has certain rights in the invention.

### SUBMISSION OF SEQUENCE LISTING AS ASCII TEXT FILE

The content of the following submission on ASCII text file is incorporated herein by reference in its entirety: a computer readable form (CRF) of the Sequence Listing (2BX5171.TXT, date recorded: Jan. 5, 2021, size: 96 KB).

### TECHNICAL FIELD

The present disclosure relates to chimeric polypeptides that include one or more Rubisco-binding motifs (RBMs) and a heterologous polypeptide. The present disclosure further relates to genetically altered plants. In particular, it relates to genetically altered plants with a chimeric polypeptide including one or more RBMs and a heterologous polypeptide. In addition, the present disclosure relates to genetically altered plants having a stabilized polypeptide including two or more RBMs and one or both of an algal Rubisco-binding membrane protein (RBMP) and a Rubisco small subunit (SSU) protein.

### BACKGROUND

Approximately one-third of global CO₂ fixation is mediated by an algal organelle called the pyrenoid (Freeman Rosenzweig et al., Cell 171: 148-162, 2017). The pyrenoid is a subcellular compartment found in the chloroplast that enhances the efficiency of photosynthesis by delivering a high concentration of CO₂ to the primary carbon-fixing enzyme Rubisco, as part of a cell-wide process termed CO₂-concentrating mechanism (CCM). Existing data suggest that the pyrenoid forms by the phase-separation of Rubisco with a linker protein (Mackinder et al., PNAS 113: 5958-5963, 2016; Wunder et al., Nat. Commun. 9: 5076, 2018). The molecular interactions underlying this condensation, however, remained unknown.

The pyrenoid represents a promising means of enhancing photosynthetic efficiency, because it does not require an enclosing membrane to be functional. Instead, the pyrenoid is composed of three sub-compartments, namely a Rubisco matrix, a means of delivering CO₂ such as thylakoid membrane tubules, and starch plates that surround the Rubisco matrix. An understanding of the assembly of each of these sub-compartments could be used to engineer a pyrenoid into plants to improve plant photosynthetic efficiency. In particular, understanding the molecular interactions that result in formation of the Rubisco matrix would be an essential first step toward engineering functional pyrenoid-like structures to improve photosynthetic efficiency in plants.

### BRIEF SUMMARY OF ASPECTS OF THE DISCLOSURE

Surprisingly, it has been found that Essential Pyrenoid Component 1 (EPYC1) of C. reinhardtii actually has ten Rubisco-binding motifs (RBMs) that bound, and linked, Rubisco. More surprisingly, it has been found that pyrenoid-associated proteins also had these RBMs. The inventors hypothesized that RBMs are hallmarks of pyrenoid proteins and that RBMs are responsible for associating these pyrenoid proteins with the pyrenoid matrix. Further, the essential amino acid residues on Rubisco that bind to the RBMs were identified through structural analysis of the interface and confirmed through mutagenesis. To prove their hypothesis and the utility of these RBMs, the inventors generated a chimeric polypeptide linking RBMs to a non-pyrenoid protein, FDX1, which resulted in the chimeric polypeptide being targeted to the pyrenoid, demonstrating that this motif can be used to target non-pyrenoid proteins to the pyrenoid and proving the hypothesis. Further, this result indicated that RBMs can be used to organize pyrenoid sub-compartments by targeting proteins. The surprising finding that RBMs are able to bind Rubisco and target pyrenoid proteins serves as the basis for many of the aspects and their various embodiments of the present disclosure.

An aspect of the disclosure includes a genetically altered higher plant or part thereof including a chimeric polypeptide including one or more Rubisco-binding motifs (RBMs) and a heterologous polypeptide. A further embodiment of this aspect includes the chimeric polypeptide including one or more, two or more, three or more, four or more, five or more, six or more, seven or more, eight or more, nine or more, or ten or more RBMs. An additional embodiment of this aspect includes the chimeric polypeptide including one or more RBMs. Yet another embodiment of this aspect includes the chimeric polypeptide including three or more RBMs. In still another embodiment of this aspect, which may be combined with any of the preceding embodiments, the one or more RBMs are independently selected from the group of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. In still another embodiment of this aspect, the one or more RBMs are independently selected from SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.

Yet another embodiment of this aspect, which may be combined with any of the preceding embodiments, includes the heterologous polypeptide being selected from a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein. A further embodiment of this aspect includes the heterologous polypeptide being the Rubisco SSU and the one or more RBMs being linked to the N-terminus or C-terminus of the Rubisco SSU, optionally through a linker polypeptide. An additional embodiment of this aspect includes the Rubisco SSU protein being an algal Rubisco SSU protein or a modified higher plant Rubisco SSU protein. In a further embodiment of this aspect, which may be combined with any of the preceding embodiments and any of the following embodiments that have the chimeric polypeptide including one or more RBMs and a heterologous polypeptide, the plant or part thereof further includes an algal Rubisco SSU protein or a modified higher plant Rubisco SSU protein. Yet another embodiment of this aspect, which may be combined with any of the preceding embodiments that have the Rubisco SSU protein, includes the Rubisco SSU protein being the algal Rubisco SSU protein. Still another embodiment of this aspect includes the algal Rubisco SSU protein being selected from the group of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. In a further embodiment of this aspect, which may be combined with any of the preceding embodiments that have the algal Rubisco SSU protein, the one or more RBMs and the algal Rubisco SSU protein are from the same algal species. In a further embodiment of this aspect, the Rubisco SSU protein is the modified higher plant Rubisco SSU protein. In an additional embodiment of this aspect, the modified higher plant Rubisco SSU includes one or more amino acid substitutions for an algal Rubisco SSU corresponding to residues 23, 24, 87, 90, 91, and 94 in SEQ ID NO: 60. In yet another embodiment of this aspect, the modified higher plant Rubisco SSU includes one or more amino acid substitutions for an algal Rubisco SSU corresponding to residues 23, 87, 90, and 94 in SEQ ID NO: 60. In yet another embodiment of this aspect that can be combined with any preceding embodiment that has the modified higher plant Rubisco SSU including one or more amino acid substitutions, the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val. Still another embodiment of this aspect includes the heterologous polypeptide being the Rubisco LSU and the one or more RBMs being linked to the N-terminus or C-terminus of the Rubisco LSU, optionally through a linker polypeptide. A further embodiment of this aspect includes the heterologous polypeptide being the membrane anchor and the membrane anchor anchoring the heterologous polypeptide to a thylakoid membrane of a chloroplast and being selected from the group of a membrane bound protein, a protein that binds to a membrane-bound protein, a transmembrane domain, or a lipidated amino acid residue in the heterologous polypeptide. An additional embodiment of this aspect includes the transmembrane domain including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 30. Yet another embodiment of this aspect includes the heterologous polypeptide being the starch binding protein and the starch binding protein including an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9.

An additional embodiment of this aspect, which may be combined with any of the preceding embodiments, includes the chimeric polypeptide being localized to a chloroplast stroma of at least one chloroplast of a plant cell of the plant or part thereof. A further embodiment of this aspect includes the plant cell being a photosynthetic cell. Yet another embodiment of this aspect includes the plant cell being a leaf mesophyll cell. In yet another embodiment of this aspect, which may be combined with any of the previous embodiments including the chimeric polypeptide being localized to a chloroplast stroma, the chimeric polypeptide is encoded by a first nucleic acid sequence and the first nucleic acid sequence is operably linked to a promoter. An additional embodiment of this aspect includes the promoter being selected from the group of a constitutive promoter, an inducible promoter, a leaf specific promoter, a mesophyll cell specific promoter, or a photosynthesis gene promoter. A further embodiment of this aspect includes the promoter being a constitutive promoter selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. Yet another embodiment of this aspect includes the promoter being a photosynthesis gene promoter selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter. Still another embodiment of this aspect, which may be combined with any previous embodiments including the first nucleic acid sequence include the first nucleic acid sequence being operably linked to a second nucleic acid sequence encoding a chloroplast transit peptide functional in the higher plant cell. In a further embodiment of this aspect, the chloroplast transit peptide is includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35. Yet another embodiment of this aspect that can be combined with any of the preceding embodiments includes the plant being a C3 crop plant. Still another embodiment of this aspect includes the C3 crop plant being selected from the group of cowpea, soybean, cassava, rice, wheat, plantain, yam, sweet potato, or potato.

An additional aspect of the disclosure includes a genetically altered higher plant or part thereof, including a polypeptide including two or more RBMs, and one or both of: an algal Rubisco-binding membrane protein (RBMP) and a Rubisco SSU protein. A further embodiment of this aspect includes the polypeptide being a stabilized polypeptide that has been modified to remove one or more chloroplastic protease cleavage sites. An additional embodiment of this aspect, which may be combined with any previous embodiments that have the polypeptide including two or more RBMs, includes the polypeptide including EPYC1 or CSP41A. Yet another embodiment of this aspect includes EPYC1 including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 52; and wherein CSP41A is selected from the group of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 68.

Yet another embodiment of this aspect, which may be combined with any previous embodiments that have the polypeptide including two or more RBMs, includes the plant or part thereof including the Rubisco SSU protein, and the Rubisco SSU protein being an algal Rubisco SSU protein or a modified higher plant Rubisco SSU protein. A further embodiment of this aspect includes the Rubisco SSU protein being the algal Rubisco SSU protein. Yet another embodiment of this aspect includes the algal Rubisco SSU protein including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. An additional embodiment of this aspect, which may be combined with any preceding aspect that has an algal Rubisco SSU protein, includes the two or more RBMs and the algal Rubisco SSU protein being from the same algal species. A further embodiment of this aspect includes the Rubisco SSU protein being the modified higher plant Rubisco SSU protein. Still another embodiment of this aspect includes the modified higher plant Rubisco SSU including one or more amino acid substitutions for an algal Rubisco SSU corresponding to residues 23, 24, 87, 90, 91, and 94 in SEQ ID NO: 60, or the modified higher plant Rubisco SSU including one or more amino acid substitutions for an algal Rubisco SSU corresponding to residues 23, 87, 90, and 94 in SEQ ID NO: 60. In a further embodiment of this aspect, the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val. In still another embodiment of this aspect, which may be combined with any of the preceding embodiments, the plant or part thereof includes the algal RBMP, and the RBMP includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 1, SEQ ID NO: 2, SEQ ID NO: 36, or SEQ ID NO: 37. An additional embodiment of this aspect, which may be combined with any of the preceding embodiments, includes the two or more RBMs independently including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NOs SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. A further embodiment of this aspect, which may be combined with any of the preceding embodiments, includes the stabilized polypeptide, the RBMP, and/or the Rubisco SSU protein being localized to a chloroplast stroma of at least one chloroplast of a plant cell of the plant or part thereof. An additional embodiment includes the plant cell being a photosynthetic cell or a leaf mesophyll cell. Yet another embodiment of this aspect, which may be combined with any of the preceding embodiments, includes the plant being a C3 crop. Still another embodiment of this aspect includes the C3 crop plant being selected from the group of cowpea, soybean, cassava, rice, wheat, plantain, yam, sweet potato, or potato.

A further aspect of the disclosure includes methods of producing the genetically altered plant of any one of the preceding embodiments that has a chimeric polypeptide including one or more RBMs and a heterologous polypeptide, including a) introducing a first nucleic acid sequence encoding a chimeric polypeptide including one or more RBMs and a heterologous polypeptide into a plant cell, tissue, or other explant; b) regenerating the plant cell, tissue, or other explant into a genetically altered plantlet; and c) growing the genetically altered plantlet into a genetically altered plant with the first nucleic acid sequence encoding the chimeric polypeptide including one or more RBMs and the heterologous polypeptide. An additional embodiment of this aspect further includes identifying successful introduction of the first nucleic acid sequence by screening or selecting the plant cell, tissue, or other explant prior to step (b); screening or selecting plantlets between step (b) and (c); or screening or selecting plants after step (c). In still another embodiment of this aspect, which may be combined with any of the preceding embodiments, transformation includes using a transformation method selected from the group of particle bombardment (i.e., biolistics, gene gun), Agrobacterium-mediated transformation, Rhizobium-mediated transformation, or protoplast transfection or transformation. Yet another embodiment of this aspect, which may be combined with any of the preceding embodiments, includes the first nucleic acid sequence being introduced with a vector. A further embodiment of this aspect includes the first nucleic acid sequence being operably linked to a promoter. An additional embodiment of this aspect includes the promoter including one or more of a constitutive promoter, an inducible promoter, a leaf specific promoter, a mesophyll cell specific promoter, or a photosynthesis gene promoter. Yet another embodiment of this aspect includes the promoter being the constitutive promoter and being selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. A further embodiment of this aspect includes the promoter being the photosynthesis gene promoter and being selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter. An additional embodiment of this aspect that may be combined with any of the preceding embodiments includes the first nucleic acid sequence being operably linked to a second nucleic acid sequence encoding a chloroplast transit peptide functional in the higher plant cell. A further embodiment of this aspect includes the chloroplast transit peptide including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35. Still another embodiment of this aspect that can be combined with any of the preceding embodiment includes the chimeric polypeptide including one or more, two or more, three or more, four or more, five or more, six or more, seven or more, eight or more, nine or more, or ten or more RBMs. An additional embodiment of this aspect includes the one or more RBMs independently including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. A further embodiment of this aspect includes the one or more RBMs being independently selected from SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID

NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.

In a further embodiment of this aspect, which may be combined with any of the preceding embodiments, the heterologous polypeptide includes a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein. A further embodiment of this aspect includes the heterologous polypeptide being the Rubisco SSU and the one or more RBMs being linked to the N-terminus or C-terminus of the Rubisco SSU, optionally through a linker polypeptide. An additional embodiment of this aspect includes the Rubisco SSU protein being an algal Rubisco SSU protein or a modified higher plant Rubisco SSU protein. Yet another embodiment of this aspect includes the Rubisco SSU protein being the algal Rubisco SSU protein, and the algal Rubisco SSU protein including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. Still another embodiment of this aspect includes the one or more RBMs and the algal Rubisco SSU protein being from the same algal species.

An additional embodiment of this aspect includes the Rubisco SSU protein being the modified higher plant Rubisco SSU protein, and the modified higher plant Rubisco SSU including one or more amino acid substitutions for an algal Rubisco SSU corresponding to residues 23, 24, 87, 90, 91, and 94 in SEQ ID NO: 60. Yet another embodiment of this aspect includes the modified higher plant Rubisco SSU including one or more amino acid substitutions for an algal Rubisco SSU corresponding to residues 23, 87, 90, and 94 in SEQ ID NO: 60. In a further embodiment of this aspect, which may be combined with any of the preceding embodiments including the modified higher plant Rubisco SSU including one or more amino acid substitutions, the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val. An additional embodiment of this aspect, which may be combined with any of the preceding embodiments including the modified higher plant Rubisco SSU including one or more amino acid substitutions, includes the vector including one or more gene editing components that target a nuclear genome sequence operably linked to a nucleic acid encoding an endogenous higher plant Rubisco SSU polypeptide. A further embodiment of this aspect includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence. In yet another embodiment of this aspect that can be combined with any preceding embodiment that includes gene editing components includes the result of gene editing being that at least part of the endogenous higher plant Rubisco SSU polypeptide is replaced with at least part of an algal Rubisco SSU polypeptide.

A further embodiment of this aspect includes the heterologous polypeptide being the Rubisco LSU and the one or more RBMs being linked to the N-terminus or C-terminus of the Rubisco LSU, optionally through a linker polypeptide. An additional embodiment of this aspect includes the heterologous polypeptide being the membrane anchor and the membrane anchor anchoring the heterologous polypeptide to a thylakoid membrane of a chloroplast and being selected from the group of a membrane bound protein, a protein that binds to a membrane-bound protein, a transmembrane domain, or a lipidated amino acid residue in the heterologous polypeptide. Still another embodiment of this aspect includes the transmembrane domain being selected from the group of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 30. Yet another embodiment of this aspect includes the heterologous polypeptide being the starch binding protein and the starch binding protein being selected from the group of an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LC19. Still another embodiment of this aspect, which may be combined with any of the preceding embodiments, further includes introducing a third nucleic acid sequence encoding an algal Rubisco SSU protein or a modified higher plant Rubisco SSU protein. A further embodiment of this aspect that can be combined with any of the preceding embodiments includes a plant or plant part produced by the method of any one of the preceding embodiments.

Yet another aspect of the disclosure includes methods of producing the genetically altered plant of any one of the preceding embodiments that has a polypeptide including two or more RBMs, including a) introducing a first nucleic acid sequence encoding a stabilized polypeptide including two or more RBMs, and introducing one or both of a second nucleic acid sequence encoding an algal RBMP and a third nucleic acid sequence encoding a Rubisco SSU protein into a plant cell, tissue, or other explant; b) regenerating the plant cell, tissue, or other explant into a genetically altered plantlet; and c) growing the genetically altered plantlet into a genetically altered plant including the first nucleic acid sequence encoding the stabilized polypeptide including two or more RBMs, and one or both of the second nucleic acid sequence encoding an algal Rubisco-binding membrane protein (RBMP) and the third nucleic acid sequence encoding a Rubisco SSU protein. An additional embodiment of this aspect includes identifying successful introduction of the first nucleic acid sequence and one or both of the second nucleic acid sequence and the third nucleic acid sequence by screening or selecting the plant cell, tissue, or other explant prior to step (b); screening or selecting plantlets between step (b) and (c); or screening or selecting plants after step (c). A further embodiment of this aspect, which may be combined with any preceding embodiment of this aspect, includes transformation including using a transformation method selected from the group of particle bombardment (i.e., biolistics, gene gun), Agrobacterium-mediated transformation, Rhizobium-mediated transformation, or protoplast transfection or transformation. Still another embodiment of this aspect, which may be combined with any preceding embodiment of this aspect, includes the first nucleic acid sequence being introduced with a first vector, the second nucleic acid sequence being introduced with a second vector, and the third nucleic acid sequence being introduced with a third vector. Yet another embodiment of this aspect includes the first nucleic acid sequence being operably linked to a first promoter, the second nucleic acid sequence being operably linked to a second promoter, and the third nucleic acid sequence being operably linked to a third promoter. A further embodiment of this aspect includes the first promoter, the second promoter, and/or the third promoter including one or more of a constitutive promoter, an inducible promoter, a leaf specific promoter, a mesophyll cell specific promoter, or a photosynthesis gene promoter. Yet another embodiment of this aspect includes the first promoter, the second promoter, and/or the third promoter being the constitutive promoter, and the constitutive promoter being selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. An additional embodiment of this aspect includes the first promoter, the second promoter, and/or the third promoter being the photosynthesis gene promoter, and the photosynthesis gene promoter being selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter.

Still another embodiment of this aspect, which may be combined with any one of the preceding embodiments, includes the first nucleic acid sequence being operably linked to a fourth nucleic acid sequence encoding a chloroplast transit peptide functional in the higher plant cell, the second nucleic acid sequence being operably linked to a fifth nucleic acid sequence encoding a chloroplast transit peptide functional in the higher plant cell, and the third nucleic acid sequence being operably linked to a sixth nucleic acid sequence encoding a chloroplast transit peptide functional in the higher plant cell. A further embodiment of this aspect includes the chloroplast transit peptide including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35. An additional embodiment of this aspect that can be combined with any preceding embodiment includes the stabilized polypeptide having been modified to remove one or more chloroplastic protease cleavage sites. Yet another embodiment of this aspect includes the stabilized polypeptide including EPYC1 or CSP41A, wherein EPYC1 includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 52; and wherein CSP41A includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 68.

An additional embodiment of this aspect that may be combined with any one of the preceding embodiments includes the third nucleic acid sequence encoding the Rubisco SSU protein being introduced in step a), and the Rubisco SSU protein being an algal Rubisco SSU protein or a modified higher plant Rubisco SSU protein. Still another embodiment of this aspect includes the Rubisco SSU protein being the algal Rubisco SSU protein, and the algal Rubisco SSU protein including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. A further embodiment of this aspect includes the two or more RBMs and the algal Rubisco SSU protein being from the same algal species. Yet another embodiment of this aspect includes the Rubisco SSU protein being the modified higher plant Rubisco SSU protein. Still another embodiment of this aspect includes the modified higher plant Rubisco SSU including one or more amino acid substitutions for an algal Rubisco SSU corresponding to residues 23, 24, 87, 90, 91, and 94 in SEQ ID NO: 60, or the modified higher plant Rubisco SSU including one or more amino acid substitutions for an algal Rubisco SSU corresponding to residues 23, 87, 90, and 94 in SEQ ID NO: 60. In an additional embodiment of this aspect, the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val. In a further embodiment of this aspect, which can be combined with any preceding embodiment that has the modified higher plant Rubisco SSU including one or more amino acid substitutions, the third vector includes one or more gene editing components that target a nuclear genome sequence operably linked to a nucleic acid encoding an endogenous higher plant Rubisco SSU polypeptide. Still another embodiment of this aspect includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence. An additional embodiment of this aspect, which can be combined with any preceding embodiment that has gene editing components, includes the result of gene editing being that at least part of the endogenous higher plant Rubisco SSU polypeptide is replaced with at least part of an algal Rubisco SSU polypeptide.

Still another embodiment of this aspect that can be combined with any one of the preceding embodiments includes the second nucleic acid sequence encoding the algal Rubisco-binding membrane protein (RBMP) being introduced in step a), and the algal RBMP including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 1, SEQ ID NO: 2, SEQ ID NO: 36, or SEQ ID NO: 37. Yet another embodiment of this aspect that can be combined with any one of the preceding embodiments includes the two or more RBMs being independently including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. A further embodiment of this aspect that can be combined with any of the preceding embodiments includes a plant or plant part produced by the method of any one of the preceding embodiments.

A further aspect of the disclosure includes methods of cultivating the genetically altered plant of any of the preceding embodiments that has a genetically altered plant, including the steps of: a) planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; b) cultivating the plant to produce harvestable seed, harvestable leaves, harvestable roots, harvestable cuttings, harvestable wood, harvestable fruit, harvestable kernels, harvestable tubers, and/or harvestable grain; and c) harvesting the harvestable seed, harvestable leaves, harvestable roots, harvestable cuttings, harvestable wood, harvestable fruit, harvestable kernels, harvestable tubers, and/or harvestable grain.

Yet another aspect of the disclosure includes chimeric polypeptides that include one or more Rubisco-binding motifs (RBMs) and a heterologous polypeptide. In examples of this aspect, the RBM includes the peptide sequence W[+]xxΨ[−] (SEQ ID NO: 28) or SEQ ID NO: 27. In other examples, the RBM includes an amino acid sequence motif including WR or WK, where the W is assigned to position ‘0’, and which motif scores 5 or higher using the following criteria: points are assigned as follows: R or K in −6 to −8: +1 point; P in −3 or −2: +1 point; D/N at −1: +1 point; optionally D/E at +2 or +3: +1 point; A/I/L/V at +4: +2 points; and D/E/COO⁻ terminus at +5: +1 point. In additional embodiments, the chimeric polypeptide includes two or more RBMs. In further embodiments, the chimeric polypeptide includes three or more RBMs. In still another embodiment of this aspect, which may be combined with any of the prior embodiments, the one or more RBMs are independently selected from the group of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. In still another embodiment of this aspect, the one or more RBMs are independently selected from SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.

In yet another chimeric polypeptide embodiment, which may be combined with any of the preceding embodiments, the heterologous polypeptide includes a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein. A further embodiment of this aspect includes the heterologous polypeptide being the Rubisco SSU and the one or more RBMs are linked to the N-terminus or C-terminus of the Rubisco SSU, optionally through a linker polypeptide. An additional embodiment of this aspect includes the Rubisco SSU protein being an algal Rubisco SSU protein or a modified higher plant Rubisco SSU protein. Yet another embodiment of this aspect includes the Rubisco SSU protein being the modified higher plant Rubisco SSU protein. In an additional embodiment of this aspect, the modified higher plant Rubisco SSU includes one or more amino acid substitutions for an algal Rubisco SSU corresponding to residues 23, 24, 87, 90, 91, and 94 in SEQ ID NO: 60. In a further embodiment, the modified higher plant Rubisco SSU includes one or more amino acid substitutions for an algal Rubisco SSU corresponding to residues 23, 87, 90, and 94 in SEQ ID NO: 60. In yet a further aspects of these chimeric polypeptide embodiment, the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val.

Still another embodiment of this aspect includes the heterologous polypeptide being the Rubisco LSU and the one or more RBMs are linked to the N-terminus or C-terminus of the Rubisco LSU, optionally through a linker polypeptide. A further embodiment of this aspect includes the heterologous polypeptide being the membrane anchor and the membrane anchor anchoring the heterologous polypeptide to a thylakoid membrane of a chloroplast and being optionally selected from the group of a membrane bound protein, a protein that binds to a membrane-bound protein, a transmembrane domain, or a lipidated amino acid residue in the heterologous polypeptide. An additional embodiment of this aspect includes the transmembrane domain including a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 30. Yet another embodiment of this aspect includes the heterologous polypeptide being the starch binding protein and the starch binding protein includes an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9.

An additional embodiment of this aspect, which may be combined with any of the preceding embodiments, includes the chimeric polypeptide being localized to a chloroplast stroma of at least one chloroplast of a plant cell of the plant or part thereof. A further embodiment of this aspect includes the plant cell being a photosynthetic cell. Yet another embodiment of this aspect includes the plant cell being a leaf mesophyll cell. In yet another embodiment of this aspect, which may be combined with any of the previous embodiments including the chimeric polypeptide being localized to a chloroplast stroma, the chimeric polypeptide is encoded by a first nucleic acid sequence and the first nucleic acid sequence is operably linked to a promoter. An additional embodiment of this aspect includes the promoter including at least one of a constitutive promoter, an inducible promoter, a leaf specific promoter, a mesophyll cell specific promoter, or a photosynthesis gene promoter. A further embodiment of this aspect includes the promoter being a constitutive promoter selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. Yet another embodiment of this aspect includes the promoter being a photosynthesis gene promoter selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter. Still another embodiment of this aspect, which may be combined with any previous embodiments including the first nucleic acid sequence includes the first nucleic acid sequence being operably linked to a second nucleic acid sequence encoding a chloroplast transit peptide functional in the higher plant cell. In a further embodiment of this aspect, the chloroplast transit peptide includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35.

Additional chimeric polypeptide embodiments include any and all of the chimeric polypeptides described herein as being expressed in a plant or plant part. Also included in the disclosure are engineered nucleic acid molecules encoding any of the chimeric polypeptides described herein.

A further aspect of the disclosure includes a synthetic pyrenoid including at least one chimeric polypeptide described herein. An additional embodiment of this aspect includes the synthetic pyrenoid being contained in a higher plant cell. Yet another embodiment of this aspect includes genetically altered higher plants or parts thereof including the higher plant cell that contains the synthetic pyrenoid. Further embodiments of this aspect include the higher plant cell being a cell of a C3 plant and/or the higher plant being a C3 plant. In still further embodiments of this aspect, inclusion of the synthetic pyrenoid in the plant cell, plant, or plant part results on CO₂ concentration in the cell, and/or results in more efficient CO₂ fixation, improved photosynthetic performance, improved cell or plant growth, and/or increased crop production.

Yet another aspect of the disclosure includes a genetically altered higher plant or part thereof, containing: an algal Rubisco SSU protein, and at least one of the following: a stabilized polypeptide including two or more RBMs; a polypeptide containing part or all of an algal Rubisco-binding membrane protein (RBMP); or one or more RBMs fused to a heterologous polypeptide that localizes to a thylakoid membrane of a chloroplast. In an additional embodiment of this aspect, the heterologous polypeptide that localizes to a thylakoid membrane of a chloroplast includes at least one of: a membrane bound protein, a protein that binds to a membrane-bound protein, a transmembrane domain, or a lipidated amino acid residue in the heterologous polypeptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show images and illustrations of the pyrenoid of Chlamydomonas reinhardtii. FIG. 1A shows an electron micrograph of a C. reinhardtii cell with anti-Rubisco immuno-gold labeling. Cells were fixed and embedded in a low viscosity epoxy resin as described in Mackinder et. al., PNAS 113: 5958-5963, 2015). Thin sectioning was performed by the Core Imaging Lab, Department of Pathology, Rutgers University, and imaging was performed at the Imaging and Analysis Center, Princeton University, on a Philips CM100 FEG with an electron beam intensity of 100 keV. FIG. 1B shows a colored electron micrograph of a C. reinhardtii cell. The region in the dashed white box (P) is enlarged and shown in the black dashed box on the right. C=chloroplast; P=pyrenoid; N=nucleus; S=starch sheath; T=thylakoid tubules; R=Rubisco matrix. FIG. 10 shows a schematic of a C. reinhardtii cell. The chloroplast and Rubisco matrix are indicated. The box on the right is a magnification of the region indicated by the dashed lines. The grey shapes represent Rubisco; the black lines represent EPYC1; the black circles on EPYC1 represent Rubisco-binding motifs (RBMs) on EPYC1.

FIGS. 2A-2B show the peptide tiling array method to identify RBMs on EPYC1. FIG. 2A shows the production of the peptide tiling array, in which peptides of 18, 22 or 25 amino acids in length tiling across the full length EPYC1 sequence were synthesized and affixed to a peptide array (full length EPYC1 sequence represented as a black line; EPYC1 peptides represented as grey and black lines; black circles represent RBMs). FIG. 2B shows an enlarged version of the region enclosed in a black dashed box in FIG. 2A, showing the Chlamydomonas reinhardtii Rubisco (grey shapes) with which the peptide arrays were incubated, peptides containing an RBM (shown in black) binding to Rubisco, and peptides that do not contain an RBM not binding to Rubisco.

FIGS. 3A-3E show the results of the peptide tiling array experiments, which identified ten RBMs on EPYC1. FIG. 3A shows an exemplary image of a peptide array following detection of binding between EPYC1 peptides on the array to Rubisco (top) or bovine serum albumin (BSA; bottom). Binding of Rubisco or BSA to the peptide array was detected using an anti-Rubisco antibody (each spot represents an EPYC1 peptide, and the darkness of each spot indicates the degree of binding of anti-Rubisco antibody to Rubisco protein or BSA that is bound to EPYC1 peptides affixed to the array). FIG. 3B shows a plot of the Rubisco-binding signal (y-axis) observed in the peptide tiling array assays across the EPYC1 amino acid sequence, with the residue position on the EPYC1 amino acid sequence indicated on the x-axis. For each residue of EPYC1, the Rubisco binding signal was averaged across peptides that included that residue. The numbers in parentheses (1-10) indicate ten RBMs on EPYC1 that exhibited strong binding to Rubisco. FIG. 3C shows the averaged binding affinity of each residue of EPYC1 of the EPYC1 amino acid sequence (SEQ ID NO: 52) as determined by the peptide tiling array results (EPYC1 repeats (Repeats 1-4) and short N- and C- termini labeled on right; shading below the sequence depicts the averaged Rubisco affinities of each residue, with dark shading indicating higher average affinity for Rubisco (see Legend)). The ten RBMs identified by the peptide tiling array experiments are indicated with numbers in parentheses beneath the sequence. The central WR residues on odd RBMs (1, 3, 5, 7, and 9) are highlighted in grey. The central WK or WR residues on even RBMs (2, 4, 6, and 8) are highlighted in grey. The central DW residues on RBM10 are highlighted in grey. FIG. 3D shows a sequence logo plot (made using weblogo.Berkeley.edu) of the consensus sequence of the even RBMs on EPYC1 (SEQ ID NO: 47). FIG. 3E shows a sequence logo plot (made using weblogo.Berkeley.edu) of the consensus sequence of the odd RBMs on EPYC1 (SEQ ID NO: 48). In FIGS. 3D-3E, the amino acid position along the RBM sequence is shown on the x-axis, the degree of conservation of an amino acid at each position along the sequence is measured in bits on the y-axis, and the size of the amino acid symbol shown at each sequence position indicates the degree of conservation (i.e., amino acids represented by tall letters are more highly conserved than amino acids represented by small letters).

FIGS. 4A-4C show the EPYC1 fragment that was used to generate the cryoelectron microscopy structure shown in FIGS. 5A-5D, as well as the binding affinity of the EPYC1 fragment for Rubisco. FIG. 4A shows a schematic of the full length EPYC1 protein sequence. The four nearly identical repeats (Repeats 1-4), flanked by short N- and C- termini are indicated. The dark grey boxes represent the ten RBMs on EPYC1. The dark grey bar above the boxes (“EPYC1 peptide”) spans RBM 2 of EPYC1 and represents the 24 amino acid EPYC1 fragment (SEQ ID NO: 51) that was used to generate the cryoelectron microscopy structure of Rubisco bound to the RBM 2 EPYC1 fragment shown in FIGS. 5A-5D. FIGS. 4B-4C provide results of SPR experiments to determine the binding affinity of the 24 amino acid EPYC1 fragment diagramed in FIG. 4A for Rubisco. FIG. 4B shows the binding affinity of the EPYC1 fragment for Rubisco as determined by SPR with the EPYC1 fragment at the indicated concentrations (0 mM, 0.25 mM, 0.5 mM, 1.0 mM, 2.0 mM, and 4.0 mM) at the times (seconds) indicated on the x-axis. The response difference (Resp. Diff., in RU) is shown on the y-axis. FIG. 4C shows the binding kinetics of the EPYC1 fragment at the concentrations (Conc.) indicated on the x-axis binding to Rubisco. The KD is circled (KD=3.09e⁻³M).

FIGS. 5A-5E show a 2.8 Å cryoelectron microscopy structure of Rubisco bound to a 24 amino acid peptide spanning RBM 2 of EPYC1, along with cartoon representations of the structure. FIG. 5A is a schematic of a Rubisco holoenzyme bound to the 24 amino acid peptide spanning RBM 2 of EPYC1, where the RBM-binding sites on the Rubisco holoenzyme are saturated with the EPYC1 peptide. FIG. 5B provides a side view of the electron density map of the EPYC1 fragment-Rubisco complex; the two boxed regions (1 and 2) are enlarged to show detail in FIGS. 6A-6B. FIG. 5C is a cartoon illustration of the side view of the density map of the EPYC1 fragment-Rubisco complex shown in FIG. 5B. FIG. 5D shows a top view of the density map of the EPYC1 fragment-Rubisco complex (image shown in FIG. 5D was rotated 90 degrees along the horizontal axis relative to the image shown in FIG. 5B). FIG. 5E is a cartoon illustration of the top view of the density map of the EPYC1 fragment-Rubisco complex shown in FIG. 5D.

For FIGS. 5B-5E, white and very light grey=Rubisco large subunit; light grey and very dark grey =Rubisco small subunit; grey=24 amino acid RBM 2 EPYC1 fragment.

FIGS. 6A-6F show detailed views of the 2.8 Å structure of Rubisco bound to the 24 amino acid RBM 2 EPYC1 fragment. FIGS. 6A-6B show EPYC1 fragments (grey with *) sitting on the two a-helices of the Rubisco small subunit (grey) (FIG. 6A is an enlargement of the view of boxed region 1 from FIG. 5A; FIG. 6B is an enlargement of the view of boxed region 2 from FIG. 5A). FIGS. 6C-6D show three salt bridge-interacting residue pairs between helices on the Rubisco SSU (dark grey; residues E24, D23, R91) and the helix of the EPYC1 peptide (grey with *; residues R64, R71, and E66). Salt bridge interactions are illustrated as dashed lines connecting two residues. Helix A and Helix B of Rubisco are indicated (dark grey). FIGS. 6E-6F show that a hydrophobic pocket is formed by one residue (L67) on the EPYC1 peptide (grey with *) and three residues (V94, L90, and M87) on one of the two helices of the Rubisco SSU (grey). Helix A and Helix B of Rubisco are indicated (dark grey).

FIG. 7 shows the interactions between the 24 amino acid EPYC1 fragment peptide spanning RBM 2 (EPYC1 peptide; SEQ ID NO: 51) that was used for cryoelectron microscopy and the Rubisco SSU Helix A (SEQ ID NO: 49) and Rubisco SSU Helix B (SEQ ID NO: 50). Rubisco SSU residues that form helices are highlighted in grey; EPYC1 residues that form a helix are highlighted in grey; residues on EPYC1 and Rubisco that are involved in the formation of salt bridges are bolded; and residues that form the hydrophobic pocket are bolded in black and italicized. Dotted lines connecting residues of EPYC1 and Rubisco SSU indicate salt-bridge forming interactions.

FIG. 8 shows a heat-map of the results of a peptide array experiment assaying the effect of substituting every amino acid in the middle 16 amino acids of the EPYC1 RBM 2 on the interaction of RBM 2 with Rubisco. The original amino acids of the EPYC1 RBM 2 (SEQ ID NO: 90) are shown along the horizontal axis, along with the corresponding residue numbers in the EPYC1 amino acid sequence (EPYC1 residues that form a helix are highlighted in grey; residues on EPYC1 that are involved in the formation of salt bridges are bolded; and residues that form the hydrophobic pocket are bolded and italicized). The amino acid substitutions that were made in the sequence of EPYC1 RBM 2 are shown on the vertical axis, along with a description of the biophysical properties of the substituting amino acid (e.g., aliphatic, aromatic, special, polar, negatively charged, and positively charged). The strength of affinity between each EPYC1 RBM2 modified peptide and Rubisco SSU (“Relative bindings”) is indicated by the color of the corresponding pixel in the heat map (white pixels denote weak or no affinity, pixels with varying shades of yellow indicate stronger affinities, and pixels with varying shades of grey to black indicate intermediate interactions).

FIGS. 9A-9C show the results of a yeast two-hybrid (Y2H) assay to measure the interaction between EPYC1 and Rubisco SSU variants. As shown in FIG. 9A, Y2H interactions were determined on yeast synthetic minimal media (SD media) lacking leucine (L) and tryptophan and histidine (H) (SD-L-W-H), where interaction strength is demonstrated by growth on increasing concentrations of the inhibitor 3-Amino-1,2,4-triazole (3-AT; growth at 20 mM 3-AT=strong interaction) (EPYC1=C. reinhardtii EPYC1; Sic, =C. reinhardtii SSU 1; “+”=positive control interaction). The images shown were taken following three days of cell growth. FIG. 9B provides a summary of the results shown in FIG. 9A. The Rubisco SSU residues that form salt bridges with EPYC1 residues are bolded (D23, E24, and R91) and the residues that form the hydrophobic pocket with EPYC1 residues are bolded and italicized (M87 and V94). The “Control” images were taken from cells grown for three days on SD-L-W media and the “Test” images were taken from cells grown for three days on SD-L-W-H with 3-AT. FIG. 9C provides a schematic summary of the Y2H results shown in FIGS. 9A-9B. Growth of yeast cells expressing the indicated EPYC1 and Rubisco SSU variants was measured after three days on SD-L-W-H with varying 3-AT concentrations. The highest concentration of 3-AT (0, 1, 2.5, 5, 10, and 20 mM) permissive for the growth of each EPYC1 and Rubisco SSU variant combination is shown, as indicated in the “Key” on the right.

FIGS. 10A-10B show the impact of mutations in EPYC1 RBMs on the formation of phase separated EPYC1-Rubisco droplets. FIG. 10A shows the amino acid sequence of EPYC1 (SEQ ID NO: 52), with the central tryptophan (W; highlighted in grey) and the central arginine or lysine (R/K; highlighted in light grey) residues of each RBM shown. FIG. 10B shows the results of phase separation experiments with or without C. reinhardtii (Cr) L8S8 Rubisco (1.875 μM) and the indicated EPYC1 protein variant (3.75 μM) in 50 mM, 100 mM or 150 mM NaCl. The EPYC1 protein variants used in each experiment are depicted on the left. Tryptophan is denoted with a black semi-circle. Lysines or arginines are denoted with grey semi-circles. In each EPYC1 protein schematic, mutation of a residue is indicated by its absence in the EPYC1 schematic. WT=wild type EPYC1; EPYC1 KR mutants (odd)=all the central R/K residues in odd RBMs were mutated to alanine; EPYC1 KR mutants (even)=all the central R/K residues in even RBMs were mutated to alanine; EPYC1 KR mutants (full)=all the central R/K residues in odd and even RBMs were mutated to alanine; EPYC1 W mutant=all the central W residues in odd and even RBMs were mutated to alanine; =no EPYC1 was used in the experiment.

FIGS. 11A-11B show results of proteomics and immunoblot experiments that identified pyrenoid proteins with RBMs. FIG. 11A shows the results of an immunoprecipitation and mass spectrometry (IP-MS) experiment identifying proteins immunoprecipitated by the anti-RBM antibody. The spectral counts of proteins immunoprecipitating with the PAP1 anti-RBM antibody in wild type (WT; x-axis) and pap1 mutant (y-axis) cell lysates are shown. Proteins of interest (RBMP1, PAP2, EPYC1, RBCL, RBMP2, CSP41A, RBCS, and PAP1) are labeled on the plot. FIG. 11B shows an anti-PAP1 immunoblot of WT, pap1 and epyc1 C. reinhardtii cell homogenates. Arrowhead, PAP1. The molecular weights of the protein bands are provided on the left in kilodaltons (kDa) (arrowheads indicate the protein bands corresponding to PAP1 and EPYC1).

FIG. 12 shows an analysis of the amino acid sequences of proteins that are immunoprecipitated by the anti-RBM antibody. On the left, the amino acid sequences of the PAP1, PAP2, RBMP1, RBMP2, EPYC1, and CSP41A are shown as horizontal lines aligned at the C-terminus (“C”) are illustrated (N-terminus denoted by an “N”). The positions of W[+]xxΨ[−] (SEQ ID NO: 28) motifs (RBMs=black circles; anti-RBM antibody depicted binding to the W[+]xxΨ[−] motifs at top), starch binding domains (black U-shapes), and transmembrane domains (black rectangles) along the amino acid sequences of the proteins are shown. The scale of the illustrations is shown by the length of the black bar, which corresponds to 100 amino acids. On the right, a sequence alignment of W[+]xxΨ[−]-motif containing regions on PAP1, PAP2, RBM P1, RBMP2, EPYC1, and CSP41A is shown (in order: SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26), as indicated by the grey connecting lines. Conserved residues are highlighted as shown in the legend: polar positively charged amino acids are indicated by blue squares (e.g., arginine and lysine), polar negatively charged amino acids are indicated by red squares (e.g., aspartic acid and glutamic acid), proline is indicated by yellow squares, aromatic amino acids are indicated by pink squares (e.g., tryptophan), non-polar amino acids are indicated by black squares (e.g., leucine, alanine, and valine), and the C-terminal carboxyl group at the end of the polypeptide is represented by red squares with the carboxyl group chemical structure

FIG. 13 shows the results of Surface Plasmon Resonance (SPR) experiments to measure the interaction between purified Rubisco and peptides containing the W[+]xxΨ[−] (SEQ ID NO: 28) motif. The peptide measured by SPR is indicated by the peptide sequence directly to the left of the graph (in order: SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26). Conserved residues are highlighted as shown in the legend: polar positively charged amino acids are indicated by blue squares (e.g., arginine and lysine), polar negatively charged amino acids are indicated by red squares (e.g., aspartic acid and glutamic acid), proline is indicated by yellow squares, aromatic amino acids are indicated by pink squares (e.g., tryptophan), non-polar amino acids are indicated by black squares (e.g., leucine, alanine, and valine), and the C-terminal carboxyl group at the end of the polypeptide is represented by red squares with the carboxyl group chemical structure. SPR binding responses were normalized to 1,000 Rubisco RUs (horizontal axis) (±SD; n=3). Non-specific binding was measured relative to three random peptides not containing the W[+]xxΨ[−] motif.

FIGS. 14A-14B show experimental methods and results of experiments to determine the effect of the W[+]xxΨ[−] (SEQ ID NO: 28) motif on FDX1 localization in C. reinhardtii cells. FIG. 14A shows fusion protein constructs that were used to test the effect of the W[+]xxΨ[−] motif on FDX1 localization in C. reinhardtii cells. To determine the normal localization of FDX1, the C-terminus of the protein was fused to the Venus fluorescent protein and a FLAG epitope tag (“Native” construct). To determine the effect of the W[+]xxΨ[−] motif on the localization of FDX1, the C-terminus of the protein was fused to the Venus fluorescent protein, a FLAG epitope tag, and three in-frame copies of the 15 C-terminal PAP2 amino acids (3X MOTIF) (“Retargeted” construct). FIG. 14B provides representative confocal fluorescence microscopy images of C. reinhardtii cells transformed with the “Native” (top row of images) or “Retargeted” FDX1 constructs (bottom row of images). The Venus fluorescent protein channel is shown in the left column, the chlorophyll autofluorescence channel is shown in the middle column, and an overlay of Venus and chlorophyll channels is shown in the right column.

FIG. 15 shows representative confocal fluorescence microscopy images of C. reinhardtii transformant cells expressing the indicated W[+]xxΨ[−] motif-containing proteins fused to the Venus fluorescent protein (i.e., PAP2-Venus, RBMP1-Venus, and RBMP2-Venus). The Venus fluorescent protein channel is shown in the left column, the chlorophyll autofluorescence channel is shown in the middle column, and an overlay of Venus and chlorophyll channels is shown in the right column.

FIGS. 16A-16B provide a model for the organization of the pyrenoid structure. FIG. 16A shows a quick-freeze deep etch electron micrograph of a low CO₂-acclimated wild type pyrenoid in C. reinhardtii. In the micrograph, circled on left is the Rubisco matrix-starch sheath interface; circled on top right is the Rubisco matrix; and circled on bottom right is the Rubisco matrix/membrane interface. The circled regions are enlarged and shown on the right of the image. FIG. 16B illustrates a model of the structure of the pyrenoid. As depicted, the Rubisco matrix is formed by the EPYC1-mediated clustering of Rubisco holoenzymes (EPYC1=black connecting lines; Rubisco=grey shapes). In addition, Rubisco-binding membrane proteins (e.g., RBMP1 and RBM P2) anchor the Rubisco matrix to tubules and starch-binding proteins (e.g., PAP1 and PAP2) enable the formation of a peripheral starch sheath.

FIGS. 17A-17D provide results of SPR experiments to determine the binding affinity for Rubisco of EPYC1 peptides used in the peptide tiling array experiments in FIGS. 3A-3E. FIG. 17A provides the binding affinity of EPYC1 peptides for Rubisco. Each EPYC1 peptide is depicted as grey solid horizontal lines spanning across the amino acid positions of the EPYC1 protein (x-axis). The y-axis provides Rubisco-binding signal measured by SPR in arbitrary units. Below the plot, the ten RBMs identified on EPYC1 are shown in circled numbers, and the EPYC1 repeats (Repeats 1-4) and short N- and C- termini are labeled on the schematic of EPYC1. FIG. 17B provides the response signal of all of the peptides (indicated on the x-axis) used in SPR experiments in FIG. 17A. The y-axis provides Rubisco-binding signal measured by SPR in arbitrary units. FIGS. 17C-17D provide comparisons of the affinity for Rubisco of EPYC1 peptides as measured by SPR (y-axis) and by the peptide array experiments described in FIGS. 3A-3E (x-axis). FIG. 17C is a scatterplot comparing the SPR Rubisco-binding signal in arbitrary units of specific regions of EPYC1 (y-axis) to the peptide tiling array raw Rubisco-binding signal in arbitrary units (x-axis). FIG. 17D is a scatterplot comparing the comparing the SPR Rubisco-binding signal in arbitrary units of specific regions of EPYC1 (y-axis) to the peptide tiling array Rubisco-binding signal running average in arbitrary units across several peptide tiling array peptides that tiled across the corresponding region on EPYC1.

FIGS. 18A-18D show the results of SPR experiments that confirmed the critical residues for interaction between EPYC1 RBM9 and Rubisco. As shown in FIG. 18A, alanine substitutions were made across the middle 16 amino acids of the EPYC1 RBM 2. The original sequence of the 16 middle amino acids of EPYC1 RBM2 is shown across the top in black (grey and black residues in SEQ ID NO: 90). FIG. 18B shows the Rubisco-binding signal measured by SPR in arbitrary units (x-axis) of the full-length peptide (SEQ ID NO: 90) and the peptides with sequence variations indicated on the y-axis (in order: SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100). FIG. 18C depicts truncations of peptides (shown as bars of different lengths with different grey shading) corresponding to the middle 16 amino acids of the EPYC1 RBM 2. The original sequence of the 16 middle amino acids of EPYC1 RBM2 is shown across the top in black (grey and black residues in SEQ ID NO: 90). FIG. 18D shows the response signals in SPR assays on the x-axis of the full-length peptide (SEQ ID NO: 90) and the peptides with sequence truncations indicated on the y-axis (in order: SEQ ID NO: 101, SEQ ID NO: 102, SEQ ID NO: 103, SEQ ID NO: 104, SEQ ID NO: 105, SEQ ID NO: 106).

FIGS. 19A-19C provide the results of peptide tiling array experiments that confirmed critical residues of EPYC1 RBM 9 for binding to Rubisco. FIG. 19A shows the full length EPYC1 protein sequence. The four nearly identical repeats (Repeats 1-4), flanked by short N- and C-termini are indicated. The dark grey boxes represent the ten RBMs on EPYC1. The grey shaded region spans RBM 9 of EPYC1 and represents a peptide that was used for peptide tiling array experiments to determine the critical residues for interaction between EPYC1 RBM 9 and Rubisco. FIG. 19B shows the averaged contribution to Rubisco binding affinity of each residue of EPYC1 (SEQ ID NO: 52) as determined by the peptide tiling array results provided in FIGS. 3A-3E (EPYC1 repeats (Repeats 1-4) and short N- and C- termini labeled on right; shading below the sequence depicts the averaged Rubisco affinities of each residue, with dark shading indicating higher average affinity for Rubisco). The boxed region corresponds to the EPYC1 peptide spanning RBM 9 shown in FIG. 19A that was used in peptide tiling array experiments to confirm critical residues of EPYC1 RBM 9 for binding to Rubisco. FIG. 19C shows a heat-map of the results of a peptide array experiment assaying the effect of substituting every amino acid in the EPYC1 RBM 9 peptide shown in FIGS. 19A-19B. The original amino acids of the EPYC1 RBM 9 are shown along the horizontal axis (SEQ ID NO: 114), along with the corresponding residue numbers in the EPYC1 amino acid sequence. The amino acid substitutions that were made in the sequence of EPYC1 RBM 9 are shown on the vertical axis, along with a description of the biophysical properties of the substituting amino acid (e.g., hydrophobic side chains (aliphatic, aromatic); special cases; polar side chains; charged side chains (negative, positive). The strength of affinity between each EPYC1 RBM 9 modified peptide and Rubisco SSU is indicated by the color of the corresponding pixel in the heat map as shown in the scale on the right (white pixels denote weak or no affinity, pixels with varying shades of yellow indicate stronger affinities, and pixels with varying shades of grey to black indicate intermediate interactions).

FIGS. 20A-20H show phylogenetic trees of green algae, protein sequences of EPYC1 and EPYC1 homologs and an alignment of the same, and sequence features of EPYC1 proteins and Rubisco SSU proteins in green algae. FIG. 20A shows a phylogenetic tree of green algal species. FIG. 20B shows evolutionary developments occurring over the course of green algal evolution as illustrated by specific green algal lineages and species. FIG. 20C shows the C. reinhardtii EPYC1 protein (SEQ ID NO: 52). FIG. 20D shows the protein sequence of the Tetrabaena socialis EPYC1 homolog (SEQ ID NO: 107). FIG. 20E shows the protein sequence of the Gonium pectorale EPYC1 homolog (SEQ ID NO: 108). FIG. 20F shows the protein sequence of the Volvox carteri f. naganensis EPYC1 homolog (SEQ ID NO: 109). FIG. 20G shows an alignment of the protein sequences of the C. reinhardtii EPYC1 protein (SEQ ID NO: 52), the T. socialis EPYC1 homolog (SEQ ID NO: 107), the G. pectorale EPYC1 homolog (SEQ ID NO: 108), and the V. carteri f. naganensis EPYC1 homolog (SEQ ID NO: 109). FIG. 20H shows a table comparing the EPYC1 RBM 2 sequence used for cryo-EM (“EPYC1 peptide for Cryo-EM”; SEQ ID NO: 90; SEQ ID NO: 110) as well as the corresponding Rubisco SSU helix A (SEQ ID NO: 50; SEQ ID NO: 111) and helix B (SEQ ID NO: 112) sequences between the listed green algal species (Chlamydomonas=C. reinhardtii; Tetrabaena=T. socialis; Gonium=G. pectorale; Volvox=V. carteri f. naganensis).

### BRIEF DESCRIPTION OF THE SEQUENCES

The nucleic acid sequences described herein and/or provided in the accompanying Sequence Listing are shown using standard letter abbreviations for nucleotide bases, as defined in 37 C.F.R. § 1.822. Only one strand of each nucleic acid sequence is shown, but the complementary strand is understood as included in embodiments where it would be appropriate. In the accompanying Sequence Listing:

SEQ ID NO: 1 is the amino acid sequence of RBMP1.

SEQ ID NO: 2 is the amino acid sequence of RBMP2.

SEQ ID NOs: 3-26 are the amino acid sequences of representative W[+]xxΨ[−]-motif containing regions.

SEQ ID NO: 27 is the overall consensus sequence of RBMs. The consensus motif emerging from the alignment of putative Rubisco binding sites is H[X1-4][P][X0-1][D/N][W][+][X2][Ψ][−], where [+]=arginine or lysine, [Xi-j]=any amino acid with a minimum number of I and a maximum number of j, [P]=proline, [D/N]=aspartic acid or asparagine, [W]=tryptophan, [4P]=alanine, isoleucine, leucine or valine, and [−]=aspartic acid, glutamic acid or carboxy terminus.

SEQ ID NO: 28 is the consensus motif W[+]xxΨ[−].

SEQ ID NOs: 29 and 30 are amino acid sequences of representative transmembrane domains.

SEQ ID NOs: 31-35 are chloroplast transit peptides.

SEQ ID NO: 36 is the amino acid sequence of the Volvox carteri homolog of RBMP1.

SEQ ID NO: 37 is the amino acid sequence of the Volvox carteri homolog of RBM P2.

SEQ ID NOs: 38-44 are amino acid sequences of representative algal Rubisco SSU proteins.

SEQ ID NOs: 45 and 47 are consensus amino acid sequences of even-numbered Rubisco-binding motifs (RBMs).

SEQ ID NOs: 46 and 48 are consensus amino acid sequences of odd-numbered RBMs.

SEQ ID NOs: 49 and 50 are amino acid sequences of rubisco SSU helix A and Helix B, respectively.

SEQ ID NO: 51 is an EPYC1 peptide.

SEQ ID NO: 52 is the amino acid sequence of Chlamydomonas reinhardtii EPYC1.

SEQ ID NOs: 53-58 are representative RBM amino acid sequences from EPYC1.

SEQ ID NO: 59 is a consensus amino acid sequence of even-numbered RBM.

SEQ ID NOs: 60 and 61 are amino acid sequences of Chlamydomonas reinhardtii Rubisco SSUs.

SEQ ID NOs: 62-67 and 69-85 are the amino acid sequences of representative RBMs.

SEQ ID NO: 68 is the amino acid sequence of Chlamydomonas reinhardtii CSP41A.

SEQ ID NO: 86 is the amino acid sequence of the C-terminal, α-helical region of Rubisco SSU.

SEQ ID NOs: 87 and 88 are peptide linkers.

SEQ ID NO: 89 is the nucleic acid sequence of the EcoRI-PfIMI digestion fragment cloned in frame into pLM005-FDX1.

SEQ ID NO: 90 is the amino acid sequence of the 16 middle amino acids of EPYC1 RBM2.

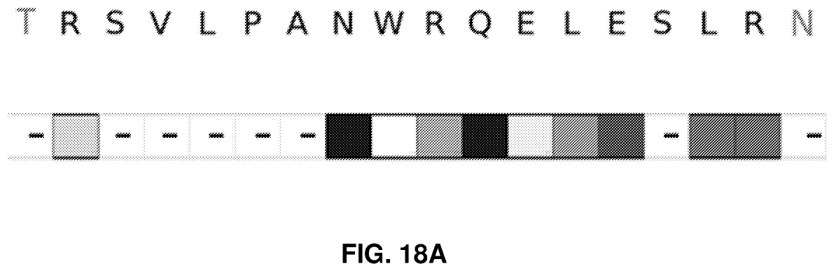

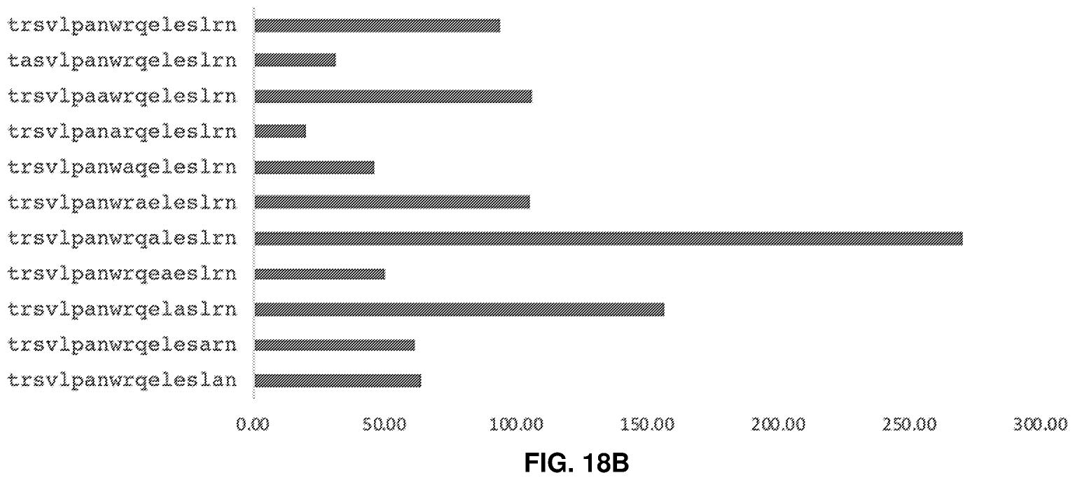

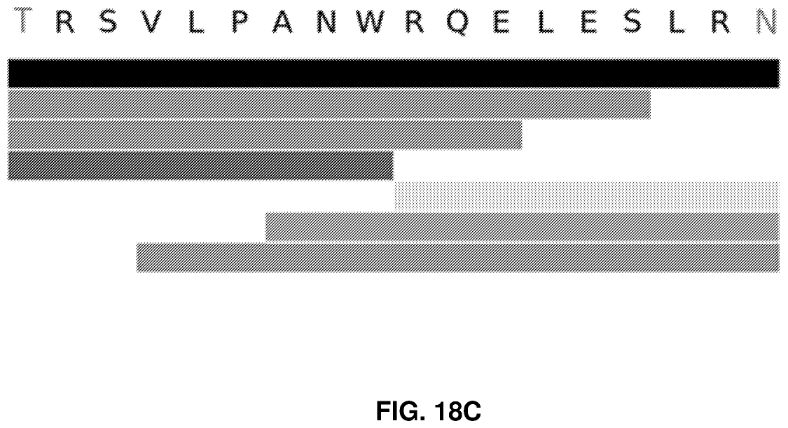

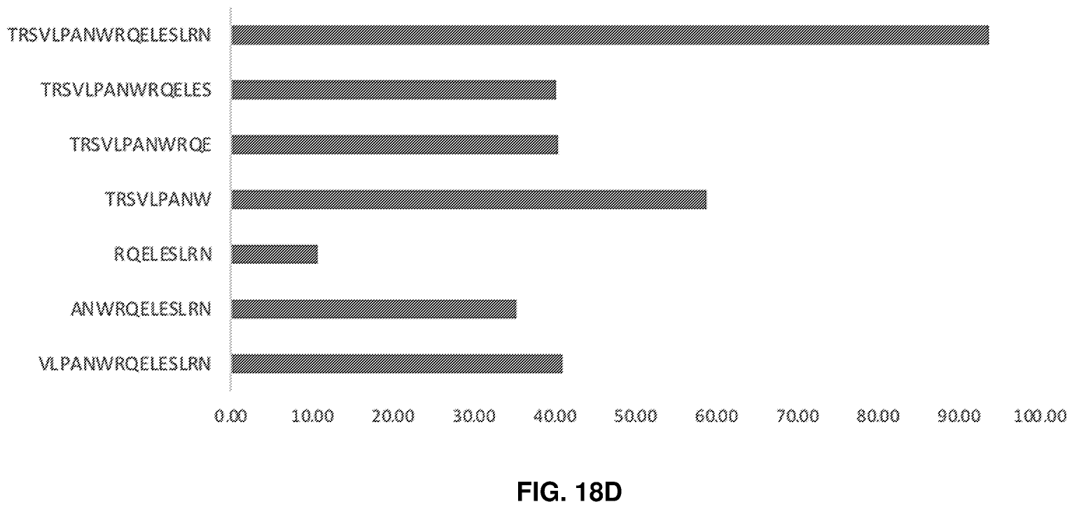

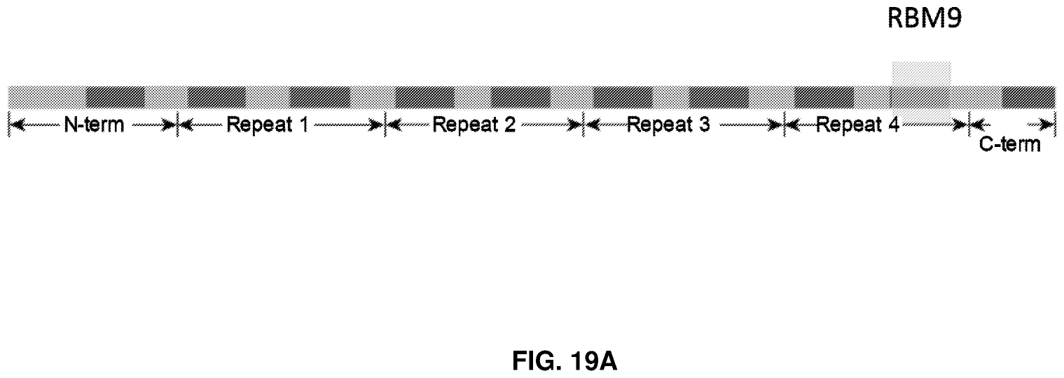

SEQ ID NOs: 91-100 are sequence variant peptides from FIG. 18B.

SEQ ID NOs: 101-106 are truncated peptides from FIG. 18D.

SEQ ID NO: 107 is the amino acid sequence of PNH11430.1, hypothetical protein TSOC_001790 [Tetrabaena socialis].

SEQ ID NO: 108 is the amino acid sequence of KXZ46518.1 hypothetical protein GPECTOR_43g955 [Gonium pectorale].

SEQ ID NO: 109 is the amino acid sequence of XP_002946604.1 hypothetical protein VOLCADRAFT_103023 [Volvox carteri f. nagariensis].

SEQ ID NO: 110 is the amino acid sequence of a Rubisco-binding region of EPYC1.

SEQ ID NOs: 111 and 112 are amino acid sequences of Rubisco SSU helix A and helix B, respectively.

SEQ ID NO: 113 is the amino acid sequence of the C-terminal region of the EPYC1 peptide.

SEQ ID NO: 114 is the amino acid sequence of EPYC1 RBM 9.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

Genetically Altered Plants: An aspect of the disclosure includes a genetically altered higher plant or part thereof including a chimeric (e.g., fusion) polypeptide including one or more Rubisco-binding motifs (RBMs) and a heterologous polypeptide. “Heterologous” in this context refers to a polypeptide that does not occur in nature joined to the RBM; in some embodiments, the heterologous polypeptide is from a different species or different organism than is the RBM. A further embodiment of this aspect includes the chimeric polypeptide includes one or more, two or more, three or more, four or more, five or more, six or more, seven or more, eight or more, nine or more, or ten or more RBMs. An additional embodiment of this aspect includes the chimeric polypeptide including one or more RBMs. Yet another embodiment of this aspect includes the chimeric polypeptide including three or more RBMs. In still another embodiment of this aspect, which may be combined with any of the preceding embodiments, the one or more RBMs are independently polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. In still another embodiment of this aspect, the one or more RBMs are independently SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.

Yet another embodiment of this aspect, which may be combined with any of the preceding embodiments, includes the heterologous polypeptide being selected from the group of a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein. A further embodiment of this aspect includes the heterologous polypeptide being the Rubisco SSU and the one or more RBMs being linked to the N-terminus or C-terminus of the Rubisco SSU, optionally through a linker polypeptide. Yet another embodiment of this aspect includes the linker polypeptide being selected from the group of polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 87 or SEQ ID NO: 88. Still another embodiment of this aspect includes the linker polypeptide being SEQ ID NO: 87 or SEQ ID NO: 88. An additional embodiment of this aspect includes the Rubisco SSU protein being an algal Rubisco SSU protein or a modified higher plant Rubisco SSU protein. In a further embodiment of this aspect, which may be combined with any of the preceding embodiments and any of the following embodiments that have the chimeric polypeptide including one or more RBMs and a heterologous polypeptide, the plant or part thereof further includes an algal Rubisco SSU protein or a modified higher plant Rubisco SSU protein. Yet another embodiment of this aspect, which may be combined with any of the preceding embodiments that have the Rubisco SSU protein, includes the Rubisco SSU protein being the algal Rubisco SSU protein. Still another embodiment of this aspect includes the algal Rubisco SSU protein being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. An additional embodiment of this aspect includes the algal Rubisco SSU protein being SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. In a further embodiment of this aspect, which may be combined with any of the preceding embodiments that have the algal Rubisco SSU protein, the one or more RBMs and the algal Rubisco SSU protein are from the same algal species. In a further embodiment of this aspect, the Rubisco SSU protein is the modified higher plant Rubisco SSU protein. In an additional embodiment of this aspect, the modified higher plant Rubisco SSU includes one or more amino acid substitutions for an algal Rubisco SSU corresponding to residues 23, 24, 87, 90, 91, and 94 in SEQ ID NO: 60. In yet another embodiment of this aspect, the modified higher plant Rubisco SSU includes one or more amino acid substitutions for an algal Rubisco SSU corresponding to residues 23, 87, 90, and 94 in SEQ ID NO: 60. In yet another embodiment of this aspect that can be combined with any preceding embodiment that has the modified higher plant Rubisco SSU including one or more amino acid substitutions, the amino acid substitution is at residue 23 and the substituted amino acid is Glu or Asp; wherein the amino acid substitution is at residue 24 and the substituted amino acid is Glu or Asp; wherein the amino acid substitution is at residue 87 and the substituted amino acid is Ala, Ile, Leu, Met, Phe, Trp, Tyr, or Val; wherein the amino acid substitution is at residue 90 and the substituted amino acid is Ala, Ile, Leu, Met, Phe, Trp, Tyr, or Val; wherein the amino acid substitution is at residue 91 and the substituted amino acid is Arg, His, or Lys; and/or wherein the amino acid substitution is at residue 94 and the substituted amino acid is Ala, Ile, Leu, Met, Phe, Trp, Tyr, or Val. In still another embodiment of this aspect that can be combined with any preceding embodiment that has the modified higher plant Rubisco SSU including one or more amino acid substitutions, the one or more RBMs and the algal Rubisco SSU protein used for the amino acid substitutions are from the same algal species. Still another embodiment of this aspect includes the heterologous polypeptide being the Rubisco LSU and the one or more RBMs are linked to the N-terminus or C-terminus of the Rubisco LSU, optionally through a linker polypeptide. Yet another embodiment of this aspect includes the linker polypeptide being selected from the group of polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 87 or SEQ ID NO: 88. Still another embodiment of this aspect includes the linker polypeptide being SEQ ID NO: 87 or SEQ ID NO: 88. A further embodiment of this aspect includes the heterologous polypeptide being the membrane anchor and the membrane anchor anchoring the heterologous polypeptide to a thylakoid membrane of a chloroplast and being selected from the group of a membrane bound protein, a protein that binds to a membrane-bound protein, a transmembrane domain, or a lipidated amino acid residue in the heterologous polypeptide. Another embodiment of this aspect includes the transmembrane domain being the transmembrane domain of PsaH (Cre07.g330250; SEQ ID NO: 29). An additional embodiment of this aspect includes the transmembrane domain being selected from the group of polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 30. A further embodiment of this aspect includes the transmembrane domain being SEQ ID NO: 30. Yet another embodiment of this aspect includes the heterologous polypeptide being the starch binding protein and the starch binding protein being selected from the group of an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9. Still another embodiment of this aspect includes the alpha-amylase/glycogenase being Cre12.g492750 or Cre12.g551200; the cyclomaltodextrin glucanotransferase being Cre16.g695800, Cre09.g394547, Cre06.g269650, or Cre06.g269601; the protein phosphatase 2C 26 being Cre03.g158050; the alpha-1,4-glucanotransferase being Cre02.g095126; the phosphoglucan, water dikinase being Cre17.g719900, Cre02.g091750, Cre10.g450500, or Cre03.g183300; the glucan 1,4-alpha-glucosidase being Cre09.g407501, Cre17.g703000, or Cre09.g415600; or the LCI9 being Cre09.g394473.

An additional embodiment of this aspect, which may be combined with any of the preceding embodiments, includes the chimeric polypeptide being localized to a chloroplast stroma of at least one chloroplast of a plant cell of the plant or part thereof. A further embodiment of this aspect includes the plant cell being a photosynthetic cell. Yet another embodiment of this aspect includes the plant cell being a leaf mesophyll cell. In yet another embodiment of this aspect, which may be combined with any of the previous embodiments including the chimeric polypeptide being localized to a chloroplast stroma, the chimeric polypeptide is encoded by a first nucleic acid sequence and the first nucleic acid sequence is operably linked to a promoter. An additional embodiment of this aspect includes the promoter being selected from the group of a constitutive promoter, an inducible promoter, a leaf specific promoter, a mesophyll cell specific promoter, or a photosynthesis gene promoter. A further embodiment of this aspect includes the promoter being a constitutive promoter selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. Yet another embodiment of this aspect includes the promoter being a photosynthesis gene promoter selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter. Still another embodiment of this aspect, which may be combined with any previous embodiments including the first nucleic acid sequence include the first nucleic acid sequence being operably linked to a second nucleic acid sequence encoding a chloroplast transit peptide functional in the higher plant cell. In a further embodiment of this aspect, the chloroplast transit peptide is a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35. An additional embodiment of this aspect includes the chloroplast transit peptide being SEQ ID NO: 31, SEQ ID NO: 32, SEQ ID NO: 33, SEQ ID NO: 34, or SEQ ID NO: 35. Yet another embodiment of this aspect, which may be combined with any of the preceding embodiments, includes the plant being any C3 plant, including C3 plants selected from the group of cowpea (e.g., black-eyed pea, catjang, yardlong bean, Vigna unguiculata), soy (e.g., soybean, soya bean, Glycine max, Glycine soja), cassava (e.g., manioc, yucca, Manihot esculenta), rice (e.g., indica rice, japonica rice, aromatic rice, glutinous rice, Oryza sativa, Oryza glaberrima), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), plantain (e.g., cooking banana, true plantain, Musa x paradisiaca, Musa spp.), yam (e.g., Dioscorea rotundata, Dioscorea cayenensis, Dioscorea alata, Dioscorea polystacha, Dioscorea bulbifera, Dioscorea esculenta, Dioscorea dumetorum, Dioscorea trifida), sweet potato (e.g., Ipomoea batatas), potato (e.g., russet potatoes, yellow potatoes, red potatoes, Solanum tuberosum), or any other C3 crop plants. In some embodiments, the plant is tobacco (i.e., Nicotiana tabacum, Nicotiana edwardsonii, Nicotiana plumbagnifolia, Nicotiana longiflora, Nicotiana benthamiana) or Arabidopsis (i.e., rockcress, thale cress, Arabidopsis thaliana).

An additional aspect of the disclosure includes a genetically altered higher plant or part thereof, including a stabilized polypeptide including two or more RBMs and one or both of an algal Rubisco-binding membrane protein (RBMP) and a Rubisco SSU protein. A further embodiment of this aspect includes the stabilized polypeptide having been modified to remove one or more chloroplastic protease cleavage sites. In provided embodiments, “stabilized” is intended to be in comparison to the stability, for instance resistance to proteolytic degradation, of a native EPYC1 or CSP41A polypeptide. An additional embodiment of this aspect, which may be combined with any previous embodiments that have the stabilized polypeptide, includes the stabilized polypeptide being selected from the group of EPYC1 or CSP41A. Yet another embodiment of this aspect includes EPYC1 being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 52, SEQ ID NO: 107, SEQ ID NO: 108, or SEQ ID NO: 109; and wherein CSP41A is selected from the group of polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 68. A further embodiment of this aspect includes EPYC1 being SEQ ID NO: 52, SEQ ID NO: 107, SEQ ID NO: 108, or SEQ ID NO: 109 and CSP41A being SEQ ID NO: 68.

Yet another embodiment of this aspect, which may be combined with any previous embodiments that have the stabilized polypeptide, includes the plant or part thereof including the Rubisco SSU protein, and the Rubisco SSU protein being an algal Rubisco SSU protein or a modified higher plant Rubisco SSU protein. A further embodiment of this aspect includes the Rubisco SSU protein being the algal Rubisco SSU protein. Yet another embodiment of this aspect includes the algal Rubisco SSU protein being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. A further embodiment of this aspect includes the algal Rubisco SSU protein being SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. An additional embodiment of this aspect, which may be combined with any preceding aspect that has an algal Rubisco SSU protein, includes the two or more RBMs and the algal Rubisco SSU protein being from the same algal species. A further embodiment of this aspect includes the Rubisco SSU protein being the modified higher plant Rubisco SSU protein. Still another embodiment of this aspect includes the modified higher plant Rubisco SSU including one or more amino acid substitutions for an algal Rubisco SSU corresponding to residues 23, 24, 87, 90, 91, and 94 in SEQ ID NO: 60, or the modified higher plant Rubisco SSU including one or more amino acid substitutions for an algal Rubisco SSU corresponding to residues 23, 87, 90, and 94 in SEQ ID NO: 60. In a further embodiment of this aspect, the amino acid substitution is at residue 23 and the substituted amino acid is Glu or Asp; wherein the amino acid substitution is at residue 24 and the substituted amino acid is Glu or Asp; wherein the amino acid substitution is at residue 87 and the substituted amino acid is Ala, Ile, Leu, Met, Phe, Trp, Tyr, or Val; wherein the amino acid substitution is at residue 90 and the substituted amino acid is Ala, Ile, Leu, Met, Phe, Trp, Tyr, or Val; wherein the amino acid substitution is at residue 91 and the substituted amino acid is Arg, His, or Lys; and/or wherein the amino acid substitution is at residue 94 and the substituted amino acid is Ala, Ile, Leu, Met, Phe, Trp, Tyr, or Val. In still another embodiment of this aspect that can be combined with any preceding embodiment that has the modified higher plant Rubisco SSU including one or more amino acid substitutions, the one or more RBMs and the algal Rubisco SSU protein used for the amino acid substitutions are from the same algal species. In still another embodiment of this aspect, which may be combined with any of the preceding embodiments, the plant or part thereof includes the algal RBMP, and the RBMP is a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 1, SEQ ID NO: 2, SEQ ID NO: 36, or SEQ ID NO: 37. A further embodiment of this aspect includes the algal RBMP being SEQ ID NO: 1, SEQ ID NO: 2, SEQ ID NO: 36, or SEQ ID NO: 37. An additional embodiment of this aspect, which may be combined with any of the preceding embodiments, includes the two or more RBMs being independently polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. Yet another embodiment of this aspect includes the two or more RBMs being SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. A further embodiment of this aspect, which may be combined with any of the preceding embodiments, includes the stabilized polypeptide, the RBMP, and/or the Rubisco SSU protein being localized to a chloroplast stroma of at least one chloroplast of a plant cell of the plant or part thereof. An additional embodiment includes the plant cell being a photosynthetic cell or a leaf mesophyll cell. Yet another embodiment of this aspect, which may be combined with any of the preceding embodiments, includes the plant being a C3 plant, including for instance a C3 plant selected from the group of cowpea (e.g., black-eyed pea, catjang, yardlong bean, Vigna unguiculata), soy (e.g., soybean, soya bean, Glycine max, Glycine soja), cassava (e.g., manioc, yucca, Manihot esculenta), rice (e.g., indica rice, japonica rice, aromatic rice, glutinous rice, Oryza sativa, Oryza glaberrima), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), plantain (e.g., cooking banana, true plantain, Musa x paradisiaca, Musa spp.), yam (e.g., Dioscorea rotundata, Dioscorea cayenensis, Dioscorea alata, Dioscorea polystacha, Dioscorea bulbifera, Dioscorea esculenta, Dioscorea dumetorum, Dioscorea trifida), sweet potato (e.g., Ipomoea batatas), potato (e.g., russet potatoes, yellow potatoes, red potatoes, Solanum tuberosum), or any other C3 crop plants. In some embodiments, the plant is tobacco (i.e., Nicotiana tabacum, Nicotiana edwardsonii, Nicotiana plumbagnifolia, Nicotiana longiflora, Nicotiana benthamiana) or Arabidopsis (i.e., rockcress, thale cress, Arabidopsis thaliana).

Methods of producing and cultivating genetically altered plants: A further aspect of the disclosure includes methods of producing the genetically altered plant of any one of the preceding embodiments that has a chimeric polypeptide including one or more RBMs and a heterologous polypeptide, including a) introducing a first nucleic acid sequence encoding a chimeric polypeptide including one or more RBMs and a heterologous polypeptide into a plant cell, tissue, or other explant; b) regenerating the plant cell, tissue, or other explant into a genetically altered plantlet; and c) growing the genetically altered plantlet into a genetically altered plant with the first nucleic acid sequence encoding the chimeric polypeptide including one or more RBMs and the heterologous polypeptide. An additional embodiment of this aspect further includes identifying successful introduction of the first nucleic acid sequence by screening or selecting the plant cell, tissue, or other explant prior to step (b); screening or selecting plantlets between step (b) and (c); or screening or selecting plants after step (c). Still another embodiment of this aspect, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation. Yet another embodiment of this aspect, which may be combined with any of the preceding embodiments, includes the first nucleic acid sequence being introduced with a vector. A further embodiment of this aspect includes the first nucleic acid sequence being operably linked to a promoter. An additional embodiment of this aspect includes the promoter being selected from the group of a constitutive promoter, an inducible promoter, a leaf specific promoter, a mesophyll cell specific promoter, or a photosynthesis gene promoter. Yet another embodiment of this aspect includes the promoter being the constitutive promoter and being selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. A further embodiment of this aspect includes the promoter being the photosynthesis gene promoter and being selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter. An additional embodiment of this aspect that may be combined with any of the preceding embodiments includes the first nucleic acid sequence being operably linked to a second nucleic acid sequence encoding a chloroplast transit peptide functional in the higher plant cell. A further embodiment of this aspect includes the chloroplast transit peptide being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35. An additional embodiment of this aspect includes the chloroplast transit peptide being SEQ ID NO: 31, SEQ ID NO: 32, SEQ ID NO: 33, SEQ ID NO: 34, or SEQ ID NO: 35. Still another embodiment of this aspect that can be combined with any of the preceding embodiment includes the chimeric polypeptide including one or more, two or more, three or more, four or more, five or more, six or more, seven or more, eight or more, nine or more, or ten or more RBMs. An additional embodiment of this aspect includes the one or more RBMs being independently polypeptides at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. A further embodiment of this aspect includes the one or more RBMs being SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.

In a further embodiment of this aspect, which may be combined with any of the preceding embodiments, the heterologous polypeptide is selected from the group of a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein. A further embodiment of this aspect includes the heterologous polypeptide being the Rubisco SSU and the one or more RBMs being linked to the N-terminus or C-terminus of the Rubisco SSU, optionally through a linker polypeptide. Yet another embodiment of this aspect includes the linker polypeptide being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 87 or SEQ ID NO: 88. Still another embodiment of this aspect includes the linker polypeptide being SEQ ID NO: 87 or SEQ ID NO: 88. An additional embodiment of this aspect includes the Rubisco SSU protein being an algal Rubisco SSU protein or a modified higher plant Rubisco SSU protein. Yet another embodiment of this aspect includes the Rubisco SSU protein being the algal Rubisco SSU protein, and the algal Rubisco SSU protein being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. An additional embodiment of this aspect includes the algal Rubisco SSU protein being SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. Still another embodiment of this aspect includes the one or more RBMs and the algal Rubisco SSU protein being from the same algal species.

An additional embodiment of this aspect includes the Rubisco SSU protein being the modified higher plant Rubisco SSU protein, and the modified higher plant Rubisco SSU including one or more amino acid substitutions for an algal Rubisco SSU corresponding to residues 23, 24, 87, 90, 91, and 94 in SEQ ID NO: 60. Yet another embodiment of this aspect includes the modified higher plant Rubisco SSU including one or more amino acid substitutions for an algal Rubisco SSU corresponding to residues 23, 87, 90, and 94 in SEQ ID NO: 60. In a further embodiment of this aspect, which may be combined with any of the preceding embodiments including the modified higher plant Rubisco SSU including one or more amino acid substitutions, the amino acid substitution is at residue 23 and the substituted amino acid is Glu or Asp; wherein the amino acid substitution is at residue 24 and the substituted amino acid is Glu or Asp; wherein the amino acid substitution is at residue 87 and the substituted amino acid is Ala, Ile, Leu, Met, Phe, Trp, Tyr, or Val; wherein the amino acid substitution is at residue 90 and the substituted amino acid is Ala, Ile, Leu, Met, Phe, Trp, Tyr, or Val; wherein the amino acid substitution is at residue 91 and the substituted amino acid is Arg, His, or Lys; and/or wherein the amino acid substitution is at residue 94 and the substituted amino acid is Ala, Ile, Leu, Met, Phe, Trp, Tyr, or Val. In still another embodiment of this aspect that can be combined with any preceding embodiment that has the modified higher plant Rubisco SSU including one or more amino acid substitutions, the one or more RBMs and the algal Rubisco SSU protein used for the amino acid substitutions are from the same algal species. An additional embodiment of this aspect, which may be combined with any of the preceding embodiments including the modified higher plant Rubisco SSU including one or more amino acid substitutions, includes the vector including one or more gene editing components that target a nuclear genome sequence operably linked to a nucleic acid encoding an endogenous higher plant Rubisco SSU polypeptide. A further embodiment of this aspect includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence. In yet another embodiment of this aspect that can be combined with any preceding embodiment that includes gene editing components includes the result of gene editing being that at least part of the endogenous higher plant Rubisco SSU polypeptide is replaced with at least part of an algal Rubisco SSU polypeptide.

A further embodiment of this aspect includes the heterologous polypeptide being the Rubisco LSU and the one or more RBMs being linked to the N-terminus or C-terminus of the Rubisco LSU, optionally through a linker polypeptide. Yet another embodiment of this aspect includes the linker polypeptide being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 87 or SEQ ID NO: 88. Still another embodiment of this aspect includes the linker polypeptide being SEQ ID NO: 87 or SEQ ID NO: 88. An additional embodiment of this aspect includes the heterologous polypeptide being the membrane anchor and the membrane anchor anchoring the heterologous polypeptide to a thylakoid membrane of a chloroplast and being selected from the group of a membrane bound protein, a protein that binds to a membrane-bound protein, a transmembrane domain, or a lipidated amino acid residue in the heterologous polypeptide. Another embodiment of this aspect includes the transmembrane domain being the transmembrane domain of PsaH (Cre07.g330250; SEQ ID NO: 29). An additional embodiment of this aspect includes the transmembrane domain being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 30. A further embodiment of this aspect includes the transmembrane domain being SEQ ID NO: 30. Yet another embodiment of this aspect includes the heterologous polypeptide being the starch binding protein and the starch binding protein being selected from the group of an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9. Still another embodiment of this aspect includes the alpha-amylase/glycogenase being Cre12.g492750 or Cre12.g551200; the cyclomaltodextrin glucanotransferase being Cre16.g695800, Cre09.g394547, Cre06.g269650, or Cre06.g269601; the protein phosphatase 2C 26 being Cre03.g158050; the alpha-1,4-glucanotransferase being Cre02.g095126; the phosphoglucan, water dikinase being Cre17.g719900, Cre02.g091750, Cre10.g450500, or Cre03.g183300; the glucan 1,4-alpha-glucosidase being Cre09.g407501, Cre17.g703000, or Cre09.g415600; or the LCI9 being Cre09.g394473.

Still another embodiment of this aspect, which may be combined with any of the preceding embodiments, further includes introducing a third nucleic acid sequence encoding an algal Rubisco SSU protein or a modified higher plant Rubisco SSU protein. Yet another embodiment of this aspect includes the Rubisco SSU protein being the algal Rubisco SSU protein, and the algal Rubisco SSU protein being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. An additional embodiment of this aspect includes the algal Rubisco SSU protein being SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. Still another embodiment of this aspect includes the one or more RBMs and the algal Rubisco SSU protein being from the same algal species. An additional embodiment of this aspect includes the Rubisco SSU protein being the modified higher plant Rubisco SSU protein, and the modified higher plant Rubisco SSU including one or more amino acid substitutions for an algal Rubisco SSU corresponding to residues 23, 24, 87, 90, 91, and 94 in SEQ ID NO: 60. Yet another embodiment of this aspect includes the modified higher plant Rubisco SSU including one or more amino acid substitutions for an algal Rubisco SSU corresponding to residues 23, 87, 90, and 94 in SEQ ID NO: 60. In a further embodiment of this aspect, which may be combined with any of the preceding embodiments including the modified higher plant Rubisco SSU including one or more amino acid substitutions, the amino acid substitution is at residue 23 and the substituted amino acid is Glu or Asp; wherein the amino acid substitution is at residue 24 and the substituted amino acid is Glu or Asp; wherein the amino acid substitution is at residue 87 and the substituted amino acid is Ala, Ile, Leu, Met, Phe, Trp, Tyr, or Val; wherein the amino acid substitution is at residue 90 and the substituted amino acid is Ala, Ile, Leu, Met, Phe, Trp, Tyr, or Val; wherein the amino acid substitution is at residue 91 and the substituted amino acid is Arg, His, or Lys; and/or wherein the amino acid substitution is at residue 94 and the substituted amino acid is Ala, Ile, Leu, Met, Phe, Trp, Tyr, or Val. In still another embodiment of this aspect that can be combined with any preceding embodiment that has the modified higher plant Rubisco SSU including one or more amino acid substitutions, the one or more RBMs and the algal Rubisco SSU protein used for the amino acid substitutions are from the same algal species. A further embodiment of this aspect that can be combined with any of the preceding embodiments includes a plant or plant part produced by the method of any one of the preceding embodiments.

Yet another aspect of the disclosure includes methods of producing the genetically altered plant of any one of the preceding embodiments that has a stabilized polypeptide including two or more RBMs, including a) introducing a first nucleic acid sequence encoding a stabilized polypeptide including two or more RBMs, and introducing one or both of a second nucleic acid sequence encoding an algal RBMP and a third nucleic acid sequence encoding a Rubisco SSU protein into a plant cell, tissue, or other explant; b) regenerating the plant cell, tissue, or other explant into a genetically altered plantlet; and c) growing the genetically altered plantlet into a genetically altered plant including the first nucleic acid sequence encoding the stabilized polypeptide including two or more RBMs, and one or both of the second nucleic acid sequence encoding an algal Rubisco-binding membrane protein (RBMP) and the third nucleic acid sequence encoding a Rubisco SSU protein. An additional embodiment of this aspect includes identifying successful introduction of the first nucleic acid sequence and one or both of the second nucleic acid sequence and the third nucleic acid sequence by screening or selecting the plant cell, tissue, or other explant prior to step (b); screening or selecting plantlets between step (b) and (c); or screening or selecting plants after step (c). A further embodiment of this aspect, which may be combined with any preceding embodiment of this aspect, includes transformation being done using a transformation method selected from the group of particle bombardment (i.e., biolistics, gene gun), Agrobacterium-mediated transformation, Rhizobium-mediated transformation, or protoplast transfection or transformation. Still another embodiment of this aspect, which may be combined with any preceding embodiment of this aspect, includes the first nucleic acid sequence being introduced with a first vector, the second nucleic acid sequence being introduced with a second vector, and the third nucleic acid sequence being introduced with a third vector. Yet another embodiment of this aspect includes the first nucleic acid sequence being operably linked to a first promoter, the second nucleic acid sequence being operably linked to a second promoter, and the third nucleic acid sequence being operably linked to a third promoter. A further embodiment of this aspect includes the first promoter, the second promoter, and/or the third promoter being the constitutive promoter, and the constitutive promoter being selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter. An additional embodiment of this aspect includes the first promoter, the second promoter, and/or the third promoter being the photosynthesis gene promoter, and the photosynthesis gene promoter being selected from the group of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, or a Calvin cycle enzyme promoter.

Still another embodiment of this aspect, which may be combined with any one of the preceding embodiments, includes the first nucleic acid sequence being operably linked to a fourth nucleic acid sequence encoding a chloroplast transit peptide functional in the higher plant cell, the second nucleic acid sequence being operably linked to a fifth nucleic acid sequence encoding a chloroplast transit peptide functional in the higher plant cell, and the third nucleic acid sequence being operably linked to a sixth nucleic acid sequence encoding a chloroplast transit peptide functional in the higher plant cell. A further embodiment of this aspect includes the chloroplast transit peptide being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NOs SEQ ID NO: 31, SEQ ID NO: 32, SEQ ID NO: 33, SEQ ID NO: 34, or SEQ ID NO: 35. Yet another embodiment of this aspect includes the chloroplast transit peptide being SEQ ID NOs SEQ ID NO: 31, SEQ ID NO: 32, SEQ ID NO: 33, SEQ ID NO: 34, or SEQ ID NO: 35. An additional embodiment of this aspect that can be combined with any preceding embodiment includes the stabilized polypeptide having been modified to remove one or more chloroplastic protease cleavage sites. Yet another embodiment of this aspect includes EPYC1 being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 52, SEQ ID NO: 107, SEQ ID NO: 108, or SEQ ID NO: 109; and wherein CSP41A is selected from the group of polypeptides having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 68. A further embodiment of this aspect includes EPYC1 being SEQ ID NO: 52, SEQ ID NO: 107, SEQ ID NO: 108, or SEQ ID NO: 109 and CSP41A being SEQ ID NO: 68.

An additional embodiment of this aspect that may be combined with any preceding embodiment includes the third nucleic acid sequence encoding the Rubisco SSU protein being introduced in step a), and the Rubisco SSU protein being an algal Rubisco SSU protein or a modified higher plant Rubisco SSU protein. Still another embodiment of this aspect includes the Rubisco SSU protein being the algal Rubisco SSU protein, and the algal Rubisco SSU protein being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. An additional embodiment of this aspect includes the algal Rubisco SSU protein being SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44. A further embodiment of this aspect includes the two or more RBMs and the algal Rubisco SSU protein being from the same algal species. Yet another embodiment of this aspect includes the Rubisco SSU protein being the modified higher plant Rubisco SSU protein. Still another embodiment of this aspect includes the modified higher plant Rubisco SSU including one or more amino acid substitutions for an algal Rubisco SSU corresponding to residues 23, 24, 87, 90, 91, and 94 in SEQ ID NO: 60, or including one or more amino acid substitutions for an algal Rubisco SSU corresponding to residues 23, 87, 90, and 94 in SEQ ID NO: 60. In an additional embodiment of this aspect, the amino acid substitution is at residue 23 and the substituted amino acid is Glu or Asp; wherein the amino acid substitution is at residue 24 and the substituted amino acid is Glu or Asp; wherein the amino acid substitution is at residue 87 and the substituted amino acid is Ala, Ile, Leu, Met, Phe, Trp, Tyr, or Val; wherein the amino acid substitution is at residue 90 and the substituted amino acid is Ala, Ile, Leu, Met, Phe, Trp, Tyr, or Val; wherein the amino acid substitution is at residue 91 and the substituted amino acid is Arg, His, or Lys; and/or wherein the amino acid substitution is at residue 94 and the substituted amino acid is Ala, Ile, Leu, Met, Phe, Trp, Tyr, or Val. In still another embodiment of this aspect that can be combined with any preceding embodiment that has the modified higher plant Rubisco SSU including one or more amino acid substitutions, the one or more RBMs and the algal Rubisco SSU protein used for the amino acid substitutions are from the same algal species. In a further embodiment of this aspect, which can be combined with any preceding embodiment that has the modified higher plant Rubisco SSU including one or more amino acid substitutions, the third vector includes one or more gene editing components that target a nuclear genome sequence operably linked to a nucleic acid encoding an endogenous higher plant Rubisco SSU polypeptide. Still another embodiment of this aspect includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence. An additional embodiment of this aspect, which can be combined with any preceding embodiment that has gene editing components, includes the result of gene editing being that at least part of the endogenous higher plant Rubisco SSU polypeptide is replaced with at least part of an algal Rubisco SSU polypeptide.

Still another embodiment of this aspect that can be combined with any one of the preceding embodiments includes the second nucleic acid sequence encoding the algal Rubisco-binding membrane protein (RBMP) being introduced in step a), and the algal RBMP being a polypeptides\ having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 1, SEQ ID NO: 2, SEQ ID NO: 36, or SEQ ID NO: 37. A further embodiment of this aspect includes the algal RBMP being SEQ ID NO: 1, SEQ ID NO: 2, SEQ ID NO: 36, or SEQ ID NO: 37. Yet another embodiment of this aspect that can be combined with any one of the preceding embodiments includes the two or more RBMs being a polypeptide having at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID

NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. Yet another embodiment of this aspect includes the two or more RBMs being SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59. A further embodiment of this aspect that can be combined with any of the preceding embodiments includes a plant or plant part produced by the method of any one of the preceding embodiments.

A further aspect of the disclosure includes methods of cultivating the genetically altered plant of any of the preceding embodiments that has a genetically altered plant, including the steps of: a) planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; b) cultivating the plant to produce harvestable seed, harvestable leaves, harvestable roots, harvestable cuttings, harvestable wood, harvestable fruit, harvestable kernels, harvestable tubers, and/or harvestable grain; and c) harvesting the harvestable seed, harvestable leaves, harvestable roots, harvestable cuttings, harvestable wood, harvestable fruit, harvestable kernels, harvestable tubers, and/or harvestable grain.

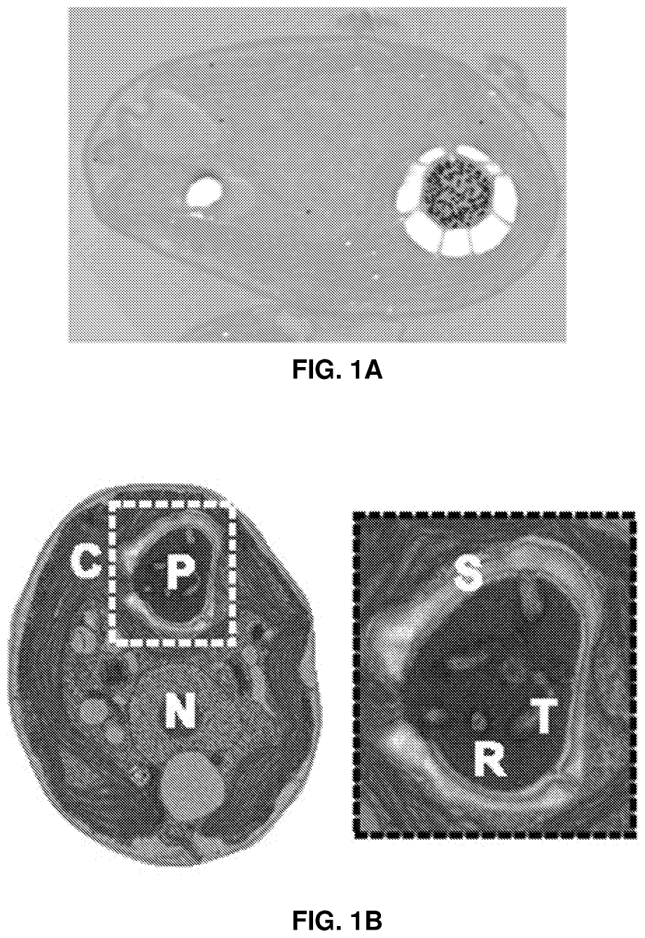

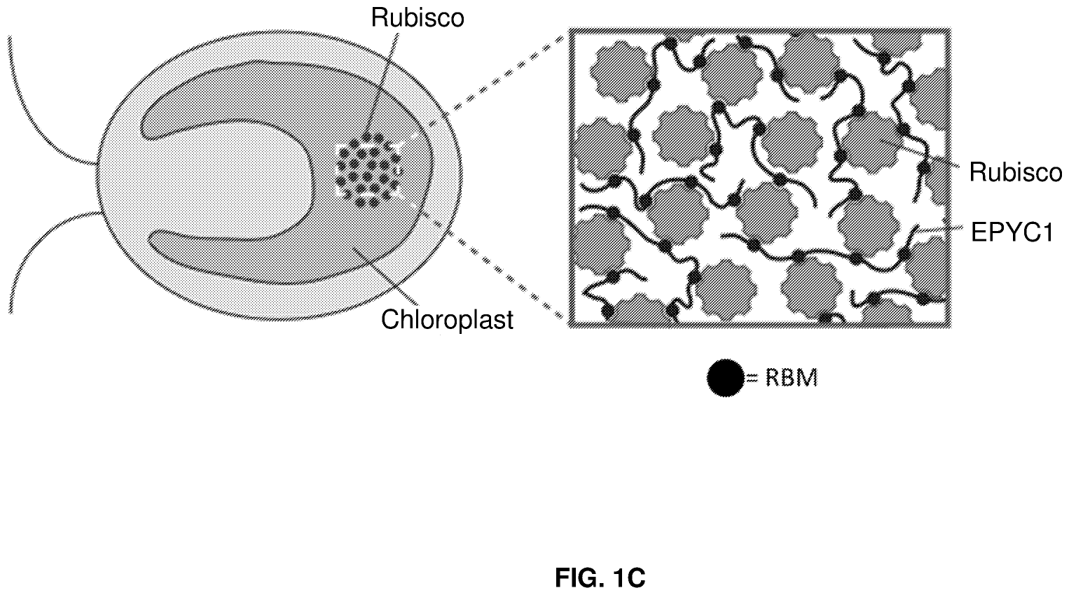

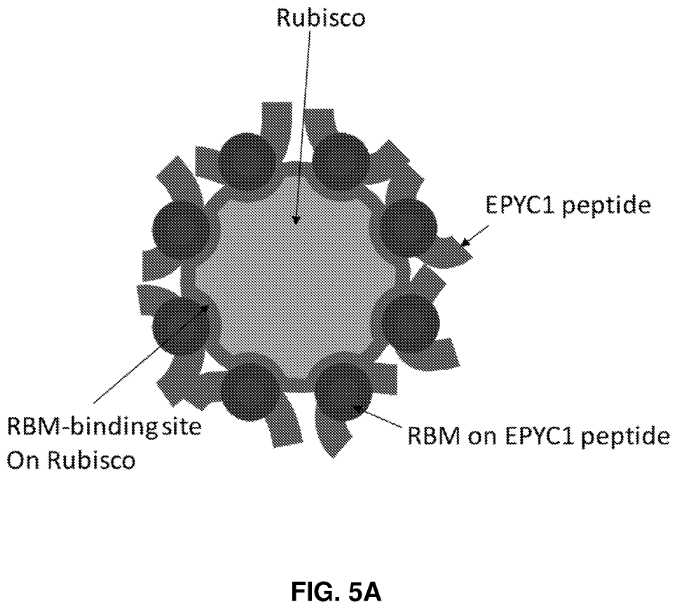

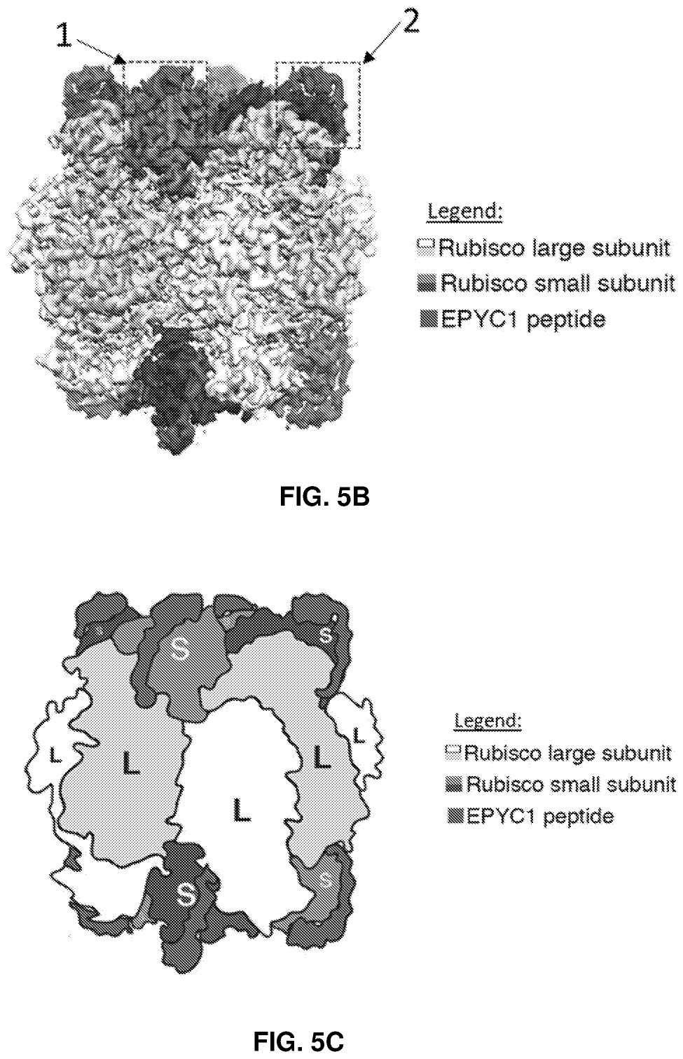

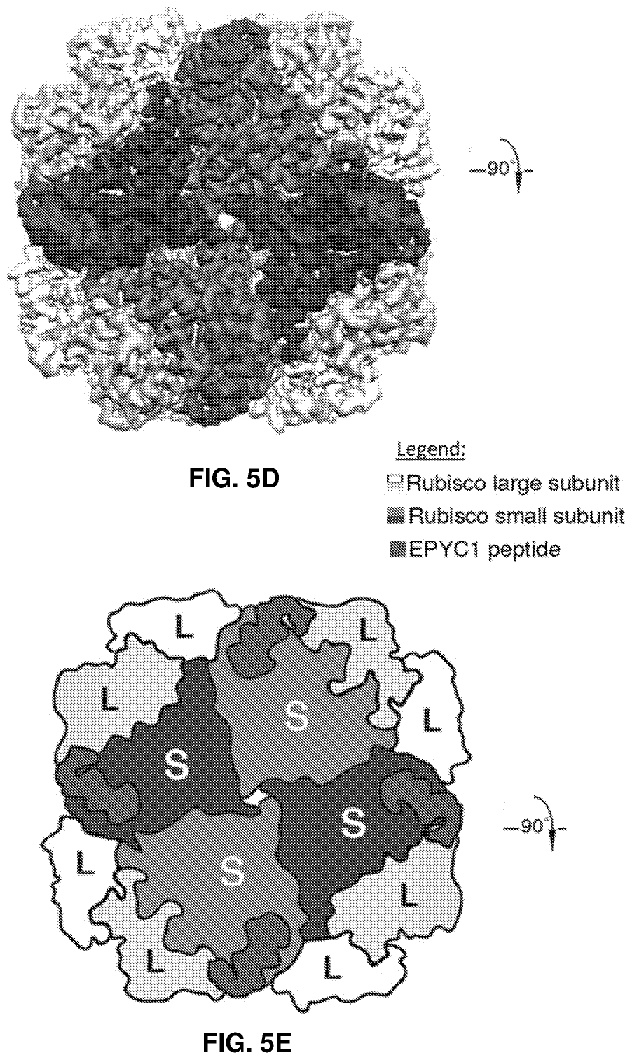

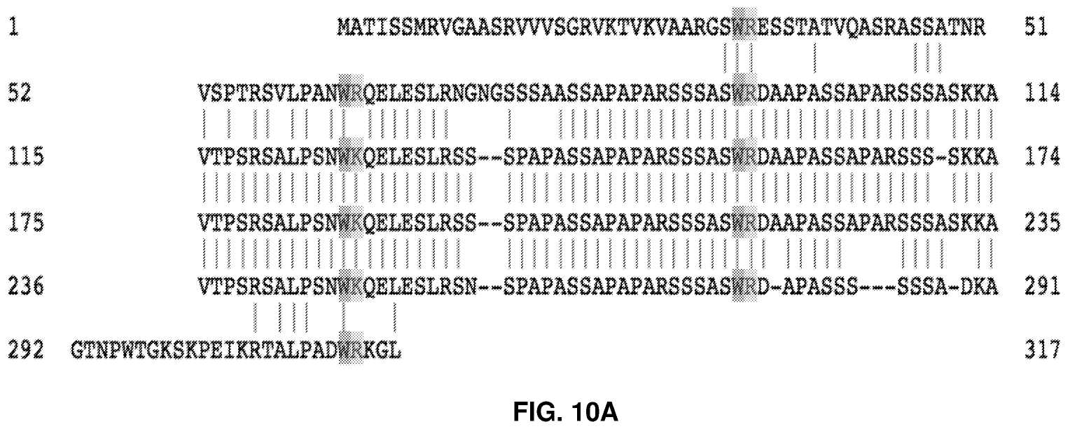

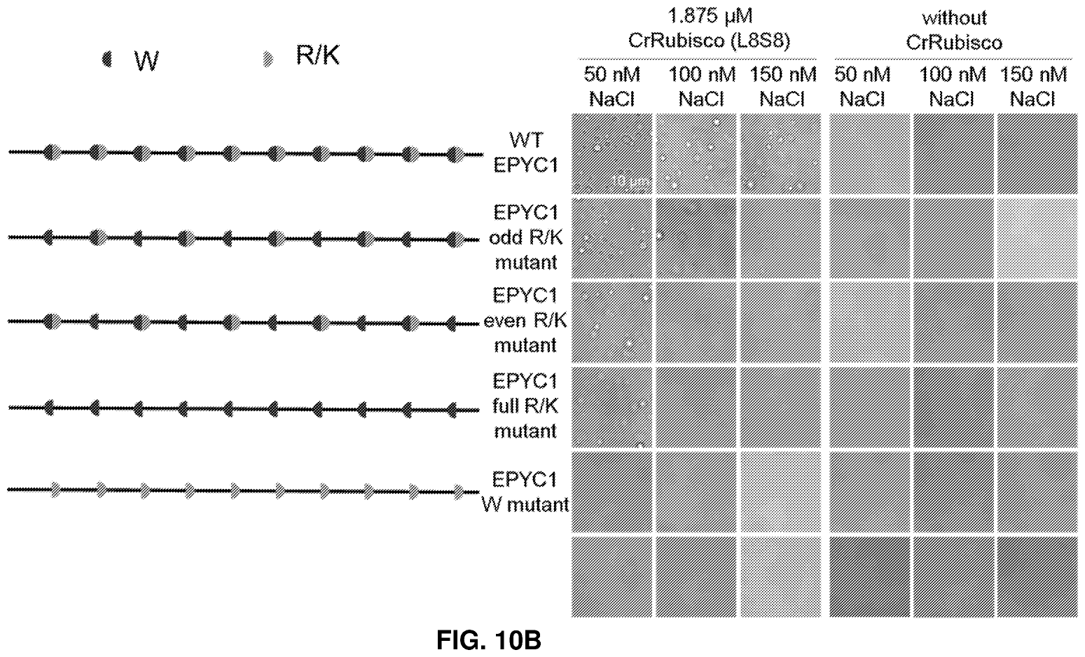

The Green Algal Pyrenoid: FIG. 1A shows an electron micrograph of a C. reinhardtii cell, in which the pyrenoid is identified by the dark spots of anti-Rubisco immuno-gold labeling. FIG. 1B shows a colored electron micrograph of a C. reinhardtii cell, in which the nucleus (N), the chloroplast (C), and the pyrenoid (P) are shown. Each of the three sub-compartments of the pyrenoid is also indicated, namely the Rubisco matrix (R), the thylakoid membrane tubules (T) that deliver CO₂, and the starch sheath (S). FIG. 10 shows a schematic of a C. reinhardtii cell, with a magnification of the Rubisco matrix. It can be seen that the RBMs on EPYC1 bind Rubisco to form the Rubisco matrix. A schematic of a Rubisco holoenzyme fully saturated with the EPYC1 polypeptide is shown in FIG. 5A.

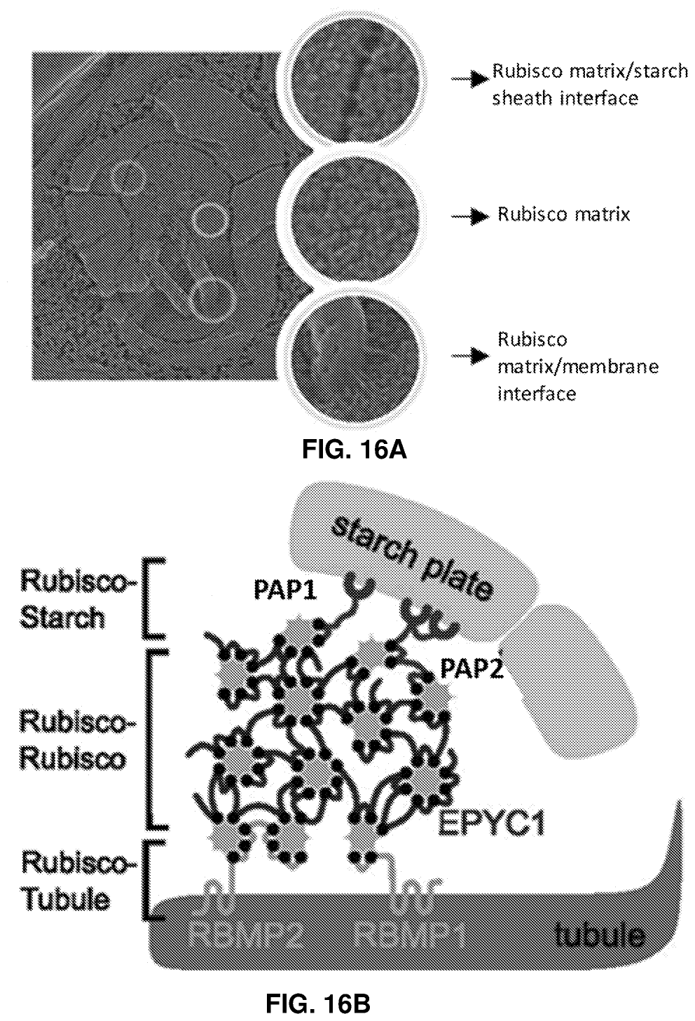

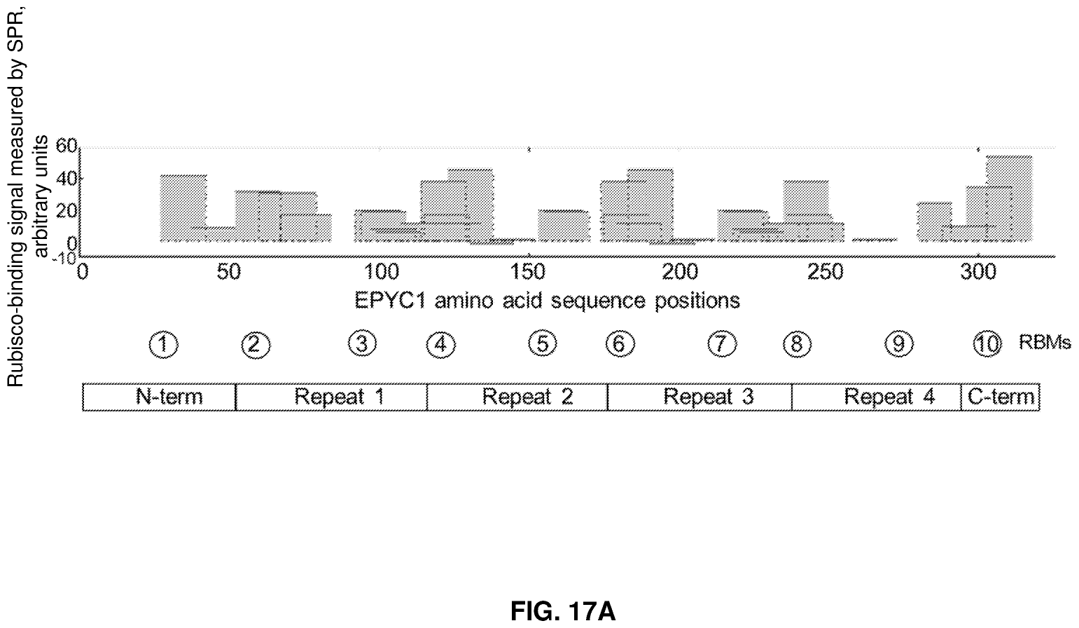

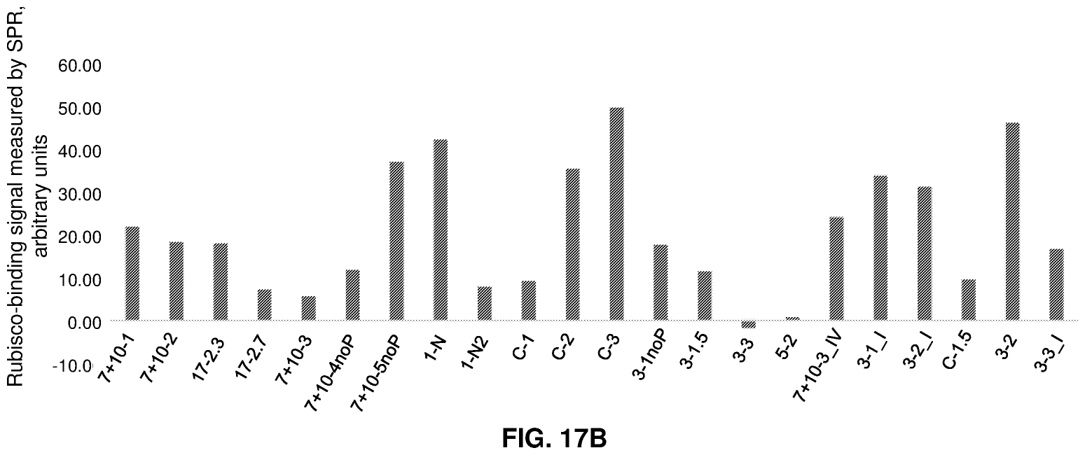

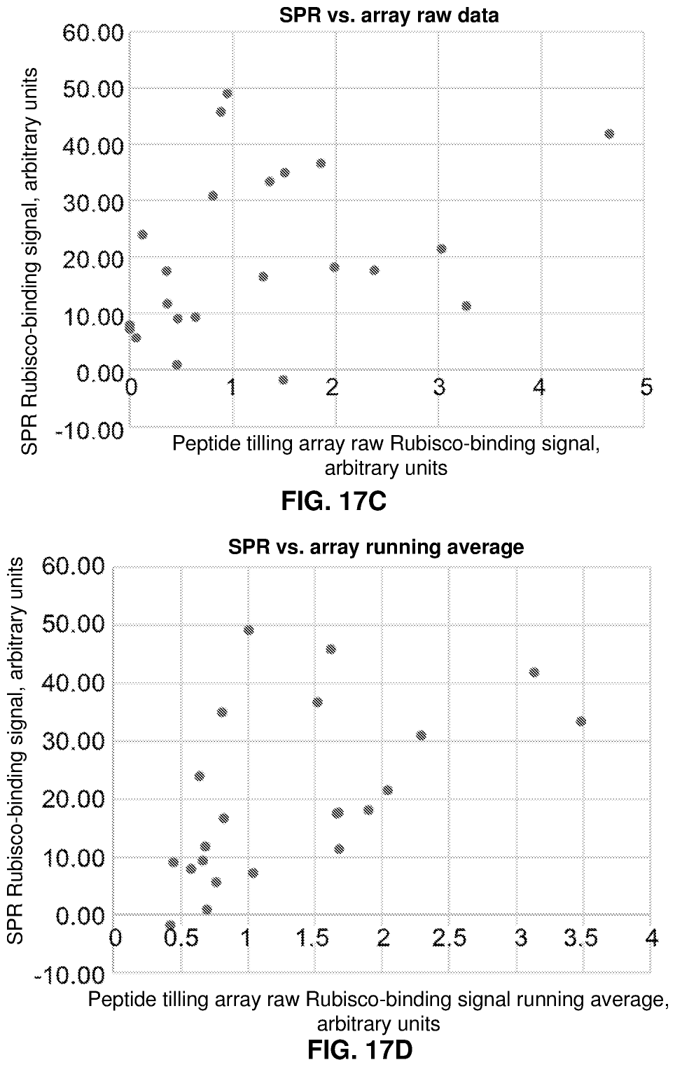

FIG. 16A shows a quick-freeze deep etch electron micrograph of a low CO₂-acclimated wild type pyrenoid in C. reinhardtii. Each of the three pyrenoid sub-compartments is indicated by a colored circle. FIG. 16B shows a cross-section of the pyrenoid sub-compartments, illustrating the role that Rubisco interactions play in each. Rubisco binds to RBMs in starch-binding proteins, EPYC1, and membrane-binding proteins. The three sub-compartments are therefore structured by these interactions.

Molecular Biological Methods to Produce Genetically Altered Plants and Plant Cells: One embodiment of the present disclosure provides a genetically altered plant or plant cell containing a chimeric polypeptide including one or more Rubisco-binding motifs (RBMs) and a heterologous polypeptide. Another embodiment of the present disclosure provides a genetically altered plant or plant cell containing a stabilized polypeptide including two or more RBMs and one or both of an algal Rubisco-binding membrane protein (RBMP) and a Rubisco SSU protein. In provided embodiments, “stabilized” is in comparison to the stability (for instance resistance to proteolytic degradation) of a native EPYC1 or CSP41A polypeptide.

In order to identify RBM motifs of the present invention, a point system may be used to identify motifs, for instance in the C. reinhardtii genome. The motifs are relative to the strictly conserved tryptophan (W), which is assigned to position ‘0’. WR or WK must be present for a sequence to be considered a potential motif. Further points are assigned as follows: R or K in −6 to −8: +1 point; P in −3 or −2: +1 point; D/N at −1: +1 point; optionally D/E at +2 or +3: +1 point; A/I/LJV at +4: +2 points; and D/E/COO⁻ terminus at +5: +1 point. Any sequence that scores 5 or more points using this system is a RBM. Hits are then ranked by decreasing order of RBM score, and homologs in the green algal lineage are searched through the BLAST search in Phytozome v.13 (Goodstein et al., Nucleic Acids Res. 40: D1178-86, 2012).

Transformation and generation of genetically altered monocotyledonous and dicotyledonous plant cells is well known in the art. See, e.g., Weising et al., Ann. Rev. Genet. 22:421-477, 1988; U.S. Pat. No. 5,679,558; Agrobacterium Protocols, ed: Gartland, Humana Press Inc. (1995); and Wang et al., Acta Hort. 461:401-408, 1998. The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and US Patent 4,684,611), plant RNA virus-mediated transformation (as described, for example in EP 0 067 553 and U.S. Pat. No. 4,407,956), liposome-mediated transformation (as described, for example in US Patent 4,536,475), and other methods such as the methods for transforming certain lines of corn (e.g., U.S. Pat. No. 6,140,553; Fromm et al., Bio/Technology 8, 833-839, 1990); Gordon-Kamm et al., The Plant Cell, 2, 603-618, 1990) and rice (Shimamoto et al., Nature, 338, 274-276, 1989; Datta et al., Bio/Technology, 8, 736-740, 1990) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, 6, 915, 1988) and Christou et al. (Trends Biotech, 8, 145, 1990) or the method of WO 00/42207.

Genetically altered plants of the present invention can be used in a conventional plant breeding scheme to produce more genetically altered plants with the same characteristics, or to introduce the genetic alteration(s) in other varieties of the same or related plant species. Seeds, which are obtained from the altered plants, in representative embodiments contain the genetic alteration(s) as a stable insert in nuclear DNA or as modifications to an endogenous gene or promoter. Plants including the genetic alteration(s) in accordance with the invention include plants containing, or derived from, root stocks of plants containing the genetic alteration(s) of the invention, e.g., fruit trees or ornamental plants. Hence, any non-transgenic grafted plant parts inserted on a transformed plant or plant part are included in the invention.

Introduced genetic elements, whether in an expression vector or expression cassette, which result in the expression of an introduced gene, will typically utilize a plant-expressible promoter. A ‘plant-expressible promoter’ as used herein refers to a promoter that ensures expression of the genetic alteration(s) of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, 9, 2871-2887, 1981), CabbB S (Franck et al., Cell 21, 285-294, 1980; Kay et al., Science, 236, 4805, 1987) and CabbB JI (Hull and Howell, Virology, 86, 482-493, 1987); cassava vein mosaic virus promoter (CsVMV); promoters from the ubiquitin family (e.g., the maize ubiquitin promoter of Christensen et al., Plant Mol Biol, 18, 675-689, 1992, or the A. thaliana UBQ10 promoter of Norris et al., Plant Mol. Biol. 21, 895-906, 1993), the gos2 promoter (de Pater et al., The Plant J 2, 834-844, 1992), the emu promoter (Last et al., Theor Appl Genet, 81, 581-588, 1990), actin promoters such as the promoter described by An et al. (The Plant J, 10, 107, 1996), the rice actin promoter described by Zhang et al. (The Plant Cell, 3, 1155-1165, 1991); promoters of the Cassava vein mosaic virus (WO 97/48819, Verdaguer et al. (Plant Mol Biol, 37, 1055-1067, 1998), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, 3, 2723 2730, 1984).

Alternatively, a plant-expressible promoter can be a tissue-specific promoter, i.e., a promoter directing a higher level of expression in some cells or tissues of the plant, e.g., in leaf mesophyll cells. In representative embodiments, leaf mesophyll specific promoters or leaf guard cell specific promoters will be used. Non-limiting examples include the leaf specific Rbcs1A promoter (A. thaliana Rubisco small subunit 1A (AT1G67090) promoter), GAPA-1 promoter (A. thaliana Glyceraldehyde 3-phosphate dehydrogenase A subunit 1 (AT3G26650) promoter), and FBA2 promoter (A. thaliana Fructose-bisphosphate aldolase 2 317 (AT4G38970) promoter) (Kromdijk et al., Science, 354(6314): 857-861, 2016). Further non-limiting examples include the leaf mesophyll specific FBPase promoter (Peleg et al., Plant J, 51(2): 165-172, 2007), the maize or rice rbcS promoter (Nomura et al., Plant Mol Biol, 44(1): 99-106, 2000), the leaf guard cell specific A. thaliana KAT1 promoter (Nakamura et al., Plant Phys, 109(2): 371-374, 1995), the A. thaliana Myrosinase-Thioglucoside glucohydrolase 1 (TGG1) promoter (Husebye et al., Plant Phys, 128(4): 1180-1188, 2002), the A. thaliana rha1 promoter (Terryn et al., Plant Cell, 5(12): 1761-1769, 1993), the A. thaliana AtCHX20 promoter (Padmanaban et al., Plant Phys, 144(1): 82-93, 2007), the A. thaliana HIC (High carbon dioxide) promoter (Gray et al., Nature, 08(6813): 713-716, 2000), the A. thaliana CYTOCHROME P450 86A2 (CYP86A2) mono-oxygenase promoter (pCYP) (Francia et al., Plant Signal & Behav, 3(9): 684-686, 2008; Galbiati et al., The Plant J, 53(5): 750-762, 2008), the potato ADP-glucose pyrophosphorylase (AGPase) promoter (Muller-Rober et al., The Plant Cell 6(5): 601-612, 1994), the grape R2R3 MYB60 transcription factor promoter (Galbiati et al., BMC Plant Bio, 11:142. doi:10.1186/1471-2229-11-142, 2011), the A. thaliana AtMYB60 promoter (Cominelli et al., Current Bio, 15(13): 1196-1200, 2005; Cominelli et al., BMC Plant Bio, 11:162. doi:10.1186/1471-2229-11-162, 2011), the A. thaliana At1g22690-promoter (pGC1) (Yang et al., Plant Methods, 4:6. doi:10.11861746-4811-4-6, 2008), and the A. thaliana AtMYB 61 promoter (Liang et al., Curr Biol, 15(13): 1201-1206, 2005). These plant promoters can be combined with enhancer elements, they can be combined with minimal promoter elements, or can include repeated elements to ensure the expression profile desired. It will also be recognized that some promoters may share two or more identifying characteristics; for instance, a single promoter may be both constitutive (expressed at all times) and cell or tissue specific (regulated by location of expression).

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present invention can be inserted in host cell DNA so that the inserted gene part is upstream (i.e., 5′) of suitable 3′ end transcription regulation signals (e.g., transcript formation and polyadenylation signals). This may be accomplished by inserting the gene in the plant cell genome (nuclear or chloroplast). Appropriate polyadenylation and transcript formation signals include those of the A. tumefaciens nopaline synthase gene (Nos terminator; Depicker et al., J. Molec Appl Gen, 1, 561-573, 1982), the octopine synthase gene (OCS terminator; Gielen et al., EMBO J, 3:835 845, 1984), the A. thaliana heat shock protein terminator (HSP terminator); the SCSV or the Malic enzyme terminators (Schunmann et al., Plant Funct Biol, 30:453-460, 2003), and the T DNA gene 7 (Velten & Schell, Nucleic Acids Res, 13, 6981-6998, 1985), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (e.g., detectable mRNA transcript or protein is produced) throughout subsequent plant generations. Stable integration into and/or editing of the nuclear genome can be accomplished by any method known in the art (e.g., microparticle bombardment, Agrobacterium-mediated transformation, CRISPR/Cas9, electroporation of protoplasts, microinjection, etc.).

The term “recombinant” or “modified” nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions. A protein encoded by a recombinant nucleic acid may be referred to as “chimeric” (literally, made of parts from different sources), particularly where the resultant amino acid sequence contains a combination two otherwise separate segments of sequence.

As used herein, the terms “overexpression” and “upregulation” refer to increased expression (e.g., of mRNA, polypeptides, etc.) relative to expression in a wild type organism (e.g., plant) as a result of genetic modification. In some embodiments, the increase in expression is a slight increase of 10% more than expression in wild type. In some embodiments, the increase in expression is an increase of 50% or more (e.g., 60%, 70%, 80%, 100%, 120%, etc.) relative to expression in wild type. In some embodiments, an endogenous gene is overexpressed. In some embodiments, an exogenous gene is overexpressed by virtue of being expressed. Overexpression of a gene in plants can be achieved through any known method in the art, including but not limited to, the use of constitutive promoters, inducible promoters, high expression promoters, enhancers, transcriptional and/or translational regulatory sequences, codon optimization, modified transcription factors, and/or mutant or modified genes that control expression of the gene to be overexpressed.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In representative embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al., Molecular Cloning: A Laboratory Manual, CSHL Press (Laboratory Manual, 1989 or Ausubel et al., Current Protocols in Molecular Biology, 1995 John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al., Science 230:1350-1354, 1985). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the BLASTN, BLASTP, and BLASTX, programs of Altschul et al. (J. Mol. Biol. 215:402-410, 1990). BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (Nucl. Acids. Res. 25:3389-3402, 1997). When utilizing BLAST and Gapped BLAST programs, the default parameters of the respective programs (BLASTN and BLASTX) are used. See resources on the World Wide Web at ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch -3, Expectation value: 10, Word size: 11, Max scores: 25, and Max alignments: 15).

Specifically contemplated host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art and which is appropriate for the particular type of cell, including without limitation, transformation, lipofection, electroporation or any other methodology known by those skilled in the art.

Plant Breeding Methods: Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (available online at lib.dr.iastate.edu under agron_books/1).

The production of double haploids can also be used for the development of homozygous lines in a breeding program. Double haploids are produced by the doubling of a set of chromosomes from a heterozygous plant to produce a completely homozygous individual. For example, see Wan et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (available online at lib.dr.iastate.edu under agron_books/1).

Synthetic Pyrenoids. With the herein described discovery of RBMs and how they function in the assembly of native algal pyrenoids, and the provision of consensus RBM sequences as well as information on where and how RBMs interact with Rubisco SSU, there are now enabled methods to exploit RBMs and their binding partners in making synthetic pyrenoids. In this context, a “synthetic pyrenoid” is a genetically engineered pyrenoid-like organelle (which is constructed through or involving some element of genetic engineering, such as expression of a chimeric protein or a protein modified as a result of gene editing), and/or a pyrenoid-like organelle that occurs in a non-natural location, such as in the cell of a higher plant cell (rather than an algal cell). Synthetic pyrenoids are characterized by one or more of the following: self-assembly of a matrix containing Rubisco (which is optionally genetically modified) and one or more proteins containing two or more RBMs (which proteins are optionally genetically modified, for instance chimeric polypeptides); self-assembly of CO₂ concentrating membrane structures associated with a Rubisco matrix; self-assembly of proteins (which are optionally genetically modified, for instance chimeric polypeptides) with starch molecules, including formation of starch granules; the ability or function of concentrating CO₂; the ability or function of improving photosynthetic performance of a cell containing the synthetic pyrenoid; the ability or function of improving productivity or growth of a cell containing the synthetic pyrenoid, or of a plant containing such a cell; and/or the ability or function of increasing crop production of plants (such as C3 plants) containing the synthetic pyrenoid.

Thus, also provided in another embodiment is a synthetic pyrenoid that includes at least one chimeric polypeptide described herein. By way of example, the synthetic pyrenoid is contained in a higher plant cell, such as a cell of a C₃ plant. Also provided are genetically altered higher plants and parts thereof, which plants contain one or more cells that contains a synthetic pyrenoid as provided herein. Genetically altered higher plants and parts thereof that contain one or more cells that contain at least one nucleic acid encoding a chimeric polypeptide, the expression of which supports or forms the synthetic pyrenoid, are also provided. In specific examples, the higher plant is a C3 plant. In various embodiments, inclusion of the synthetic pyrenoid in the plant cell, plant, or plant part results in CO₂ concentration in the cell, and/or results in more efficient CO₂ fixation, improved photosynthetic performance, improved cell or plant growth, and/or increased crop production.

First Set of Exemplary Embodiments
1. A genetically altered higher plant or part thereof, comprising a chimeric polypeptide comprising one or more Rubisco-binding motifs (RBMs) and a heterologous polypeptide.
2. The plant or part thereof of embodiment 1, wherein the chimeric polypeptide includes one or more, two or more, three or more, four or more, five or more, six or more, seven or more, eight or more, nine or more, or ten or more RBMs.
3. The plant or part thereof of embodiment 2, wherein the chimeric polypeptide includes one or more RBMs.
4. The plant or part thereof of embodiment 2, wherein the chimeric polypeptide includes three or more RBMs.
5. The plant or part thereof of any one of embodiments 1-4, wherein the one or more RBMs are independently selected from the group consisting of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
6. The plant or part thereof of embodiment 5, wherein the one or more RBMs are independently selected from SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
7. The plant or part thereof of any one of embodiments 1-6, wherein the heterologous polypeptide includes a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein.
8. The plant or part thereof of embodiment 7, wherein the heterologous polypeptide is the Rubisco SSU and the one or more RBMs are linked to the N-terminus or C-terminus of the Rubisco SSU, optionally through a linker polypeptide.
9. The plant or part thereof of embodiment 8, wherein the Rubisco SSU protein is an algal Rubisco SSU protein or a modified higher plant Rubisco SSU protein.
10. The plant or part thereof of any one of embodiments 1-8, wherein the plant or part thereof further includes an algal Rubisco SSU protein or a modified higher plant Rubisco SSU protein.
11. The plant or part thereof of embodiment 9 or embodiment 10, wherein the Rubisco SSU protein is the algal Rubisco SSU protein.
12. The plant or part thereof of embodiment 11, wherein the algal Rubisco SSU protein includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44.
13. The plant or part thereof of embodiment 11 or embodiment 12, wherein the one or more RBMs and the algal Rubisco SSU protein are from the same algal species.
14. The plant or part thereof of embodiment 9 or embodiment 10, wherein the Rubisco SSU protein is the modified higher plant Rubisco SSU protein.
15. The plant or part thereof of embodiment 14, wherein the modified higher plant Rubisco SSU includes one or more amino acid substitutions for an algal Rubisco SSU corresponding to residues 23, 24, 87, 90, 91, and 94 in SEQ ID NO: 60.
16. The plant or part thereof of embodiment 14 or embodiment 15, wherein the modified higher plant Rubisco SSU includes one or more amino acid substitutions for an algal Rubisco SSU corresponding to residues 23, 87, 90, and 94 in SEQ ID NO: 60.
17. The plant or part thereof of embodiment 15 or embodiment 16, wherein: the amino acid substitution is at residue 23 and the substituted amino acid is Glu or Asp;

the amino acid substitution is at residue 24 and the substituted amino acid is Glu or Asp;

the amino acid substitution is at residue 87 and the substituted amino acid is Ala, Ile, Leu, Met, Phe, Trp, Tyr, or Val;

the amino acid substitution is at residue 90 and the substituted amino acid is Ala, Ile, Leu, Met, Phe, Trp, Tyr, or Val;

the amino acid substitution is at residue 91 and the substituted amino acid is Arg, His, or Lys; and/or

the amino acid substitution is at residue 94 and the substituted amino acid is Ala, Ile, Leu, Met, Phe, Trp, Tyr, or Val.
18. The plant or part thereof of embodiment 7, wherein the heterologous polypeptide is the Rubisco LSU and the one or more RBMs are linked to the N-terminus or C-terminus of the Rubisco LSU, optionally through a linker polypeptide.
19. The plant or part thereof of embodiment 7, wherein the heterologous polypeptide is the membrane anchor and the membrane anchor anchors the heterologous polypeptide to a thylakoid membrane of a chloroplast and is optionally selected from the group consisting of a membrane bound protein, a protein that binds to a membrane-bound protein, a transmembrane domain, and a lipidated amino acid residue in the heterologous polypeptide.
20. The plant or part thereof of embodiment 19, wherein the transmembrane domain includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 30.
21. The plant or part thereof of embodiment 7, wherein the heterologous polypeptide is the starch binding protein and the starch binding protein includes an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9.
22. The plant or part thereof of any one of embodiments 1-21, wherein the chimeric polypeptide is localized to a chloroplast stroma of at least one chloroplast of a plant cell of the plant or part thereof.
23. The plant or part thereof of embodiment 22, wherein the plant cell is a photosynthetic cell.
24. The plant or part thereof of embodiment 23, wherein the photosynthetic cell is a leaf mesophyll cell.
25. The plant or part thereof of any one of embodiments 22-24, wherein the chimeric polypeptide is encoded by a first nucleic acid sequence, and the first nucleic acid sequence is operably linked to a promoter.
26. The plant or part thereof of embodiment 25, wherein the promoter includes at least one of a constitutive promoter, an inducible promoter, a leaf specific promoter, a mesophyll cell specific promoter, or a photosynthesis gene promoter.
27. The plant or part thereof of embodiment 26, wherein the promoter is a constitutive promoter selected from the group consisting of a CaMV35S promoter, a derivative of the CaMV35S promoter, a maize ubiquitin promoter, an actin promoter, a trefoil promoter, a vein mosaic cassava virus promoter, and an A. thaliana UBQ10 promoter.
28. The plant or part thereof of embodiment 26, wherein the promoter is a photosynthesis gene promoter selected from the group consisting of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, and a Calvin cycle enzyme promoter.
29. The plant or part thereof of any one of embodiments 25-28, wherein the first nucleic acid sequence is operably linked to a second nucleic acid sequence encoding a chloroplastic transit peptide functional in the higher plant cell.
30. The plant or part thereof of embodiment 29, wherein the chloroplast transit peptide includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35.
31. The plant or part thereof of any one of embodiments 1-30, wherein the plant is a C3 crop plant.
32. The plant or part thereof of embodiment 31, wherein the C3 crop plant selected from the group consisting of cowpea, soybean, cassava, rice, wheat, plantain, yam, sweet potato, and potato.
33. A genetically altered higher plant or part thereof, including: a polypeptide including two or more RBMs, and one or both of: an algal Rubisco-binding membrane protein (RBMP); and a Rubisco SSU protein.
34. The plant or part thereof of embodiment 33, wherein the polypeptide is a stabilized polypeptide that has been modified to remove one or more chloroplastic protease cleavage sites.
35. The plant or part thereof of embodiment 33 or embodiment 34, wherein the polypeptide includes EPYC1 or CSP41A.
36. The plant or part thereof of embodiment 35, wherein EPYC1 includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 52; and wherein CSP41A includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 68.
37. The plant or part thereof of any one of embodiments 32-36, wherein the plant or part thereof includes the Rubisco SSU protein, and wherein the Rubisco SSU protein is an algal Rubisco SSU protein or a modified higher plant Rubisco SSU protein.
38. The plant or part thereof of embodiment 37, wherein the Rubisco SSU protein is the algal Rubisco SSU protein.
39. The plant or part thereof of embodiment 38, wherein the algal Rubisco SSU protein includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44.
40. The plant or part thereof of embodiment 38 or embodiment 39, wherein the two or more RBMs and the algal Rubisco SSU protein are from the same algal species.
41. The plant or part thereof of embodiment 37, wherein the Rubisco SSU protein is the modified higher plant Rubisco SSU protein.
42. The plant or part thereof of embodiment 41, wherein the modified higher plant Rubisco SSU includes one or more amino acid substitutions for an algal Rubisco SSU corresponding to residues 23, 24, 87, 90, 91, and 94 in SEQ ID NO: 60, or wherein the modified higher plant Rubisco SSU includes one or more amino acid substitutions for an algal Rubisco SSU corresponding to residues 23, 87, 90, and 94 in SEQ ID NO: 60.
43. The plant or part thereof of embodiment 42, wherein: the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val.
44. The plant or part thereof of any one of embodiments 32-43, wherein the plant or part thereof includes the algal RBMP, and wherein the RBMP includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 1, SEQ ID NO: 2, SEQ ID NO: 36, or SEQ ID NO: 37.
45. The plant or part thereof of any one of embodiments 32-44, wherein the two or more RBMs independently include a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
46. The plant or part thereof of any one of embodiments 32-45, wherein the stabilized polypeptide, the RBMP, and/or the Rubisco SSU protein are localized to a chloroplast stroma of at least one chloroplast of a plant cell of the plant or part thereof.
47. The plant or part thereof of embodiment 46, wherein the plant cell is a photosynthetic cell or a leaf mesophyll cell.
48. The plant or part thereof of any one of embodiments 32-47, wherein the plant is a C3 crop plant.
49. The plant or part thereof of embodiment 48, wherein the C3 crop plant is selected from the group consisting of cowpea, soybean, cassava, rice, wheat, plantain, yam, sweet potato, and potato.
50. A method of producing the genetically altered plant of any one of embodiments 1-31, including: a) introducing a first nucleic acid sequence encoding a chimeric polypeptide including one or more RBMs and a heterologous polypeptide into a plant cell, tissue, or other explant; b) regenerating the plant cell, tissue, or other explant into a genetically altered plantlet; and c) growing the genetically altered plantlet into a genetically altered plant including the first nucleic acid sequence encoding the chimeric polypeptide including one or more RBMs and the heterologous polypeptide.
51. The method of embodiment 50, further including identifying successful introduction of the first nucleic acid sequence by: screening or selecting the plant cell, tissue, or other explant prior to step (b); screening or selecting plantlets between step (b) and (c); and/or screening or selecting plants after step (c).
52. The method of embodiment 50 or embodiment 51, wherein transformation includes using a transformation method selected from the group consisting of particle bombardment (i.e., biolistics, gene gun), Agrobacterium-mediated transformation, Rhizobium-mediated transformation, and protoplast transfection or transformation.
53. The method of any one of embodiments 51-52, wherein the first nucleic acid sequence is introduced with a vector.
54. The method of embodiment 53, wherein the first nucleic acid sequence is operably linked to a promoter.
55. The method of embodiment 54, wherein the promoter includes one or more of a constitutive promoter, an inducible promoter, a leaf specific promoter, a mesophyll cell specific promoter, or a photosynthesis gene promoter.
56. The method of embodiment 55, wherein the promoter is the constitutive promoter selected from the group consisting of a CaMV35S promoter, a derivative of the CaMV35S promoter, a maize ubiquitin promoter, an actin promoter, a trefoil promoter, a vein mosaic cassava virus promoter, and an A. thaliana UBQ10 promoter.
57. The method of embodiment 55, wherein the promoter is the photosynthesis gene promoter selected from the group consisting of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, and a Calvin cycle enzyme promoter.
58. The method of any one of embodiments 54-57, wherein the first nucleic acid sequence is operably linked to a second nucleic acid sequence encoding a chloroplastic transit peptide functional in the higher plant cell.
59. The method of embodiment 58, wherein the chloroplast transit peptide includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35.
60. The method of any one of embodiments 50-59, wherein the chimeric polypeptide includes one or more, two or more, three or more, four or more, five or more, six or more, seven or more, eight or more, nine or more, or ten or more RBMs.
61. The method of embodiment 60, wherein the one or more RBMs independently include a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
62. The method of embodiment 61, wherein the one or more RBMs are independently selected from SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
63. The method of any one of embodiments 50-62, wherein the heterologous polypeptide includes a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein.
64. The method of embodiment 63, wherein the heterologous polypeptide is the Rubisco SSU and the one or more RBMs are linked to the N-terminus or C-terminus of the Rubisco SSU, optionally through a linker polypeptide.
65. The method of embodiment 64, wherein the Rubisco SSU protein is an algal Rubisco SSU protein or a modified higher plant Rubisco SSU protein.
66. The method of embodiment 65, wherein the Rubisco SSU protein is the algal Rubisco SSU protein, and wherein the algal Rubisco SSU protein includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44.
67. The method of embodiment 66, wherein the one or more RBMs and the algal Rubisco SSU protein are from the same algal species.
68. The plant or part thereof of embodiment 65, wherein the Rubisco SSU protein is the modified higher plant Rubisco SSU protein, and wherein the modified higher plant Rubisco SSU includes one or more amino acid substitutions for an algal Rubisco SSU corresponding to residues 23, 24, 87, 90, 91, and 94 in SEQ ID NO: 60.
69. The method of embodiment 68, wherein the modified higher plant Rubisco SSU includes one or more amino acid substitutions for an algal Rubisco SSU corresponding to residues 23, 87, 90, and 94 in SEQ ID NO: 60.
70. The method of embodiment 68 or embodiment 69, wherein: the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val.
71. The method of any one of embodiments 68-70, wherein the vector includes one or more gene editing components that target a nuclear genome sequence, operably linked to a nucleic acid encoding an endogenous higher plant Rubisco SSU polypeptide.
72. The method of embodiment 71, wherein one or more gene editing components are selected from the group consisting of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; and a vector including a CRISPR/Cas enzyme encoding sequence and a targeting sequence, wherein the targeting sequence targets the nuclear genome sequence.
73. The method of embodiment 71 or embodiment 72, wherein the result of gene editing is that at least part of the endogenous higher plant Rubisco SSU polypeptide is replaced with at least part of an algal Rubisco SSU polypeptide.
74. The method of embodiment 63, wherein the heterologous polypeptide is the Rubisco LSU and the one or more RBMs are linked to the N-terminus or C-terminus of the Rubisco LSU, optionally through a linker polypeptide.
75. The method of embodiment 63, wherein the heterologous polypeptide is the membrane anchor and the membrane anchor anchors the heterologous polypeptide to a thylakoid membrane of a chloroplast and is optionally selected from the group consisting of a membrane bound protein, a protein that binds to a membrane-bound protein, a transmembrane domain, and a lipidated amino acid residue in the heterologous polypeptide.
76. The method of embodiment 75, wherein the transmembrane domain includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 30.
77. The method of embodiment 63, wherein the heterologous polypeptide is the starch binding protein and the starch binding protein includes an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9.
78. The method of any one of embodiments 50-77, further including introducing a third nucleic acid sequence encoding an algal Rubisco SSU protein or a modified higher plant Rubisco SSU protein.
79. A plant or plant part produced by the method of any one of embodiments 50-78.
80. A method of producing the genetically altered plant of any one of embodiments 32-49, including:
a) introducing a first nucleic acid sequence encoding a stabilized polypeptide including two or more RBMs, and introducing one or both of a second nucleic acid sequence encoding an algal RBMP and a third nucleic acid sequence encoding a Rubisco SSU protein into a plant cell, tissue, or other explant; b) regenerating the plant cell, tissue, or other explant into a genetically altered plantlet; and c) growing the genetically altered plantlet into a genetically altered plant including the first nucleic acid sequence encoding the stabilized polypeptide including two or more RBMs, and one or both of the second nucleic acid sequence encoding an algal Rubisco-binding membrane protein (RBMP) and the third nucleic acid sequence encoding a Rubisco SSU protein.
81. The method of embodiment 80, further including identifying successful introduction of the first nucleic acid sequence and one or both of the second nucleic acid sequence and the third nucleic acid sequence by: screening or selecting the plant cell, tissue, or other explant prior to step (b); screening or selecting plantlets between step (b) and (c); or screening or selecting plants after step (c).
82. The method of embodiment 80 or embodiment 81, wherein transformation includes using a transformation method selected from the group consisting of particle bombardment (i.e., biolistics, gene gun), Agrobacterium-mediated transformation, Rhizobium-mediated transformation, and protoplast transfection or transformation.
83. The method of any one of embodiments 80-82, wherein the first nucleic acid sequence is introduced with a first vector, the second nucleic acid sequence is introduced with a second vector, and the third nucleic acid sequence is introduced with a third vector.
84. The method of embodiment 83, wherein the first nucleic acid sequence is operably linked to a first promoter, the second nucleic acid sequence is operably linked to a second promoter, and the third nucleic acid sequence is operably linked to a third promoter.
85. The method of embodiment 84, wherein the first promoter, the second promoter, and the third promoter independently include one or more of a constitutive promoter, an inducible promoter, a leaf specific promoter, a mesophyll cell specific promoter, or a photosynthesis gene promoter.
86. The method of embodiment 85, wherein the first promoter, the second promoter, and/or the third promoter are the constitutive promoter, and wherein the constitutive promoter is selected from the group consisting of a CaMV35S promoter, a derivative of the CaMV35S promoter, a maize ubiquitin promoter, an actin promoter, a trefoil promoter, a vein mosaic cassava virus promoter, and an A. thaliana UBQ10 promoter.
87. The method of embodiment 85, wherein the first promoter, the second promoter, and/or the third promoter are the photosynthesis gene promoter, and wherein the photosynthesis gene promoter is selected from the group consisting of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, and a Calvin cycle enzyme promoter.
88. The method of any one of embodiments 83-87, wherein the first nucleic acid sequence is operably linked to a fourth nucleic acid sequence encoding a chloroplastic transit peptide functional in the higher plant cell, the second nucleic acid sequence is operably linked to a fifth nucleic acid sequence encoding a chloroplastic transit peptide functional in the higher plant cell, and the third nucleic acid sequence is operably linked to a sixth nucleic acid sequence encoding a chloroplastic transit peptide functional in the higher plant cell.
89. The plant or part thereof of embodiment 88, wherein the chloroplast transit peptide includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35.
90. The method of any one of embodiments 80-89, wherein the stabilized polypeptide has been modified to remove one or more chloroplastic protease cleavage sites.
91. The method of embodiment 90, wherein the stabilized polypeptide includes EPYC1 or CSP41A, wherein EPYC1 includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 52; and wherein CSP41A includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 68.
92. The method of any one of embodiments 80-91, wherein the third nucleic acid sequence encoding the Rubisco SSU protein was introduced in step a), and wherein the Rubisco SSU protein is an algal Rubisco SSU protein or a modified higher plant Rubisco SSU protein.
93. The method of embodiment 92, wherein the Rubisco SSU protein is the algal Rubisco SSU protein, and wherein the algal Rubisco SSU protein includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 60, SEQ ID NO: 61, SEQ ID NO: 38, SEQ ID NO: 39, SEQ ID NO: 40, SEQ ID NO: 41, SEQ ID NO: 42, SEQ ID NO: 43, or SEQ ID NO: 44.
94. The method of embodiment 93, wherein the two or more RBMs and the algal Rubisco SSU protein are from the same algal species.
95. The method of embodiment 92, wherein the Rubisco SSU protein is the modified higher plant

Rubisco SSU protein.
96. The method of embodiment 95, wherein the modified higher plant Rubisco SSU includes one or more amino acid substitutions for an algal Rubisco SSU corresponding to residues 23, 24, 87, 90, 91, and 94 in SEQ ID NO: 60, or wherein the modified higher plant Rubisco SSU includes one or more amino acid substitutions for an algal Rubisco SSU corresponding to residues 23, 87, 90, and 94 in SEQ ID NO: 60.
97. The method of embodiment 96, wherein: the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val.
98. The method of any one of embodiments 95-97, wherein the third vector includes one or more gene editing components that target a nuclear genome sequence operably linked to a nucleic acid encoding an endogenous higher plant Rubisco SSU polypeptide.
99. The method of embodiment 98, wherein one or more gene editing components are selected from the group consisting of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; and a vector including a CRISPR/Cas enzyme encoding sequence and a targeting sequence, wherein the targeting sequence targets the nuclear genome sequence.
100. The method of embodiment 98 or embodiment 99, wherein the result of gene editing is that at least part of the endogenous higher plant Rubisco SSU polypeptide is replaced with at least part of an algal Rubisco SSU polypeptide.
101. The method of any one of embodiments 80-100, wherein the second nucleic acid sequence encoding the algal RBMP was introduced in step a), and wherein the algal RBMP includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 1, SEQ ID NO: 2, SEQ ID NO: 36, or SEQ ID NO: 37.
102. The method of any one of embodiments 80-101, wherein the two or more RBMs independently include a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NOs SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
103. A plant or plant part produced by the method of any one of embodiments 80-102.
104. A method of cultivating the genetically altered plant of any one of embodiments 1-49, 79, and 103, including: planting a genetically altered seedling, a genetically altered plantlet, a genetically altered cutting, a genetically altered tuber, a genetically altered root, or a genetically altered seed in soil to produce the genetically altered plant, or grafting the genetically altered seedling, the genetically altered plantlet, or the genetically altered cutting to a root stock or a second plant grown in soil to produce the genetically altered plant; cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain.
105. A chimeric polypeptide including one or more Rubisco-binding motifs (RBMs) and a heterologous polypeptide.
106. The chimeric polypeptide of embodiment 105, wherein the RBM includes the peptide sequence W[+]xxΨ[−] (SEQ ID NO: 28) or SEQ ID NO: 29.
107. The chimeric polypeptide of embodiment 105, wherein the RBM includes an amino acid sequence motif including WR or WK, where the W is assigned to position ‘0’, and which motif scores 5 or higher using the following criteria: points are assigned as follows: R or K in −6 to −8: +1 point; P in -3 or -2: +1 point; D/N at -1: +1 point; optionally D/E at +2 or +3: +1 point; A/I/LJV at +4: +2 points; and D/E/COO⁻ terminus at +5: +1 point.
108. The chimeric polypeptide of any one of embodiments 105-107, wherein the chimeric polypeptide includes two or more RBMs.
109. The chimeric polypeptide of any one of embodiments 105-107, wherein the chimeric polypeptide includes three or more RBMs.
110. The chimeric polypeptide of any one of embodiments 105-109, wherein the one or more RBMs are independently selected from the group consisting of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
111. The chimeric polypeptide of embodiment 110, wherein the one or more RBMs are independently selected from SEQ ID NO: 53, SEQ ID NO: 54, SEQ ID NO: 55, SEQ ID NO: 56, SEQ ID NO: 57, SEQ ID NO: 58, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 62, SEQ ID NO: 63, SEQ ID NO: 64, SEQ ID NO: 65, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 28, SEQ ID NO: 45, SEQ ID NO: 46, SEQ ID NO: 47, SEQ ID NO: 48, or SEQ ID NO: 59.
112. The chimeric polypeptide of any one of embodiments 105-110, wherein the heterologous polypeptide includes a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein.
113. The chimeric polypeptide of embodiment 112, wherein the heterologous polypeptide is the Rubisco SSU and the one or more RBMs are linked to the N-terminus or C-terminus of the Rubisco SSU, optionally through a linker polypeptide.
114. The chimeric polypeptide of embodiment 113, wherein the Rubisco SSU protein is an algal Rubisco SSU protein or a modified higher plant Rubisco SSU protein.
115. The chimeric polypeptide of embodiment 114, wherein the Rubisco SSU protein is the modified higher plant Rubisco SSU protein.
116. The chimeric polypeptide of embodiment 115, wherein the modified higher plant Rubisco SSU includes one or more amino acid substitutions for an algal Rubisco SSU corresponding to residues 23, 24, 87, 90, 91, and 94 in SEQ ID NO: 60.
117. The chimeric polypeptide of embodiment 115 or embodiment 116, wherein the modified higher plant Rubisco SSU includes one or more amino acid substitutions for an algal Rubisco SSU corresponding to residues 23, 87, 90, and 94 in SEQ ID NO: 60.
118. The chimeric polypeptide of embodiment 116 or embodiment 117, wherein: the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val.
119. The chimeric polypeptide of embodiment 112, wherein the heterologous polypeptide is the Rubisco LSU and the one or more RBMs are linked to the N-terminus or C-terminus of the Rubisco LSU, optionally through a linker polypeptide.
120. The chimeric polypeptide of embodiment 112, wherein the heterologous polypeptide is the membrane anchor and the membrane anchor anchors the heterologous polypeptide to a thylakoid membrane of a chloroplast and is optionally selected from the group consisting of a membrane bound protein, a protein that binds to a membrane-bound protein, a transmembrane domain, and a lipidated amino acid residue in the heterologous polypeptide.
121. The chimeric polypeptide of embodiment 120, wherein the transmembrane domain includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 30.
122. The chimeric polypeptide of embodiment 112, wherein the heterologous polypeptide is the starch binding protein and the starch binding protein includes an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9.
123. The chimeric polypeptide of any one of embodiments 105-122, wherein the chimeric polypeptide is localized to a chloroplast stroma of at least one chloroplast of a plant cell of the plant or part thereof.
124. The chimeric polypeptide of embodiment 123, wherein the plant cell is a photosynthetic cell.
125. The chimeric polypeptide of embodiment 124, wherein the photosynthetic cell is a leaf mesophyll cell.
126. The chimeric polypeptide of any one of embodiments 123-125, wherein the chimeric polypeptide is encoded by a first nucleic acid sequence, and the first nucleic acid sequence is operably linked to a promoter.
127. The chimeric polypeptide of embodiment 126, wherein the promoter includes at least one of a constitutive promoter, an inducible promoter, a leaf specific promoter, a mesophyll cell specific promoter, or a photosynthesis gene promoter.
128. The chimeric polypeptide of embodiment 127, wherein the promoter is a constitutive promoter selected from the group consisting of a CaMV35S promoter, a derivative of the CaMV35S promoter, a maize ubiquitin promoter, an actin promoter, a trefoil promoter, a vein mosaic cassava virus promoter, and an A. thaliana UBQ10 promoter.
129. The chimeric polypeptide of embodiment 127, wherein the promoter is a photosynthesis gene promoter selected from the group consisting of a Photosystem I promoter, a Photosystem II promoter, a b6f promoter, an ATP synthase promoter, a sedoheptulose-1,7-bisphosphatase (SBPase) promoter, a fructose-1,6-bisphosphate aldolase (FBPA) promoter, and a Calvin cycle enzyme promoter.
130. The chimeric polypeptide of any one of embodiments 126-129, wherein the first nucleic acid sequence is operably linked to a second nucleic acid sequence encoding a chloroplastic transit peptide functional in the higher plant cell.
131. The chimeric polypeptide of embodiment 130, wherein the chloroplast transit peptide includes a polypeptide having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 31, SEQ ID NO: 32, SEQ ID NO: 33, SEQ ID NO: 34, or SEQ ID NO: 35.
132. A synthetic pyrenoid including at least one chimeric polypeptide described herein.
133. The synthetic pyrenoid of embodiment 132, contained in a higher plant cell.
134. A genetically altered higher plant or part thereof, including the higher plant cell of embodiment 133.
135. A genetically altered higher plant or part thereof, including: an algal Rubisco SSU protein, and at least one of the following: a stabilized polypeptide including two or more RBMs; a polypeptide containing part or all of an algal Rubisco-binding membrane protein (RBMP); or one or more RBMs fused to a heterologous polypeptide that localizes to a thylakoid membrane of a chloroplast.
136. The genetically altered higher plant or part thereof of embodiment 135, wherein the heterologous polypeptide that localizes to a thylakoid membrane of a chloroplast includes at least one of: a membrane bound protein, a protein that binds to a membrane-bound protein, a transmembrane domain, or a lipidated amino acid residue in the heterologous polypeptide.

Second Set of Exemplary Embodiments
1. A genetically altered higher plant or part thereof, including: a stabilized polypeptide including two or more RBMs, or a chimeric polypeptide including one or more Rubisco-binding motifs (RBMs) and a heterologous polypeptide, and a Rubisco SSU protein, wherein the Rubisco SSU protein is an algal Rubisco SSU protein or a modified higher plant Rubisco SSU protein that includes one or more amino acid substitutions for an algal Rubisco SSU corresponding to residues 23, 24, 87, 90, 91, and 94 in SEQ ID NO: 60.
2. A genetically altered higher plant or part thereof, including a chimeric polypeptide including one or more Rubisco-binding motifs (RBMs) and a heterologous polypeptide.
3. The plant or part thereof of embodiment 1 or embodiment 2, wherein the one or more RBMs are independently selected from the group consisting of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 27 or SEQ ID NO: 28.
4. The plant or part thereof of any one of embodiments 1-3, wherein the heterologous polypeptide includes a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein.
5. The plant or part thereof of embodiment 4, wherein the heterologous polypeptide is the Rubisco SSU and the one or more RBMs are linked to the N-terminus or C-terminus of the Rubisco SSU, optionally through a linker polypeptide.
6. The plant or part thereof of any one of embodiments 2-5, wherein the plant or part thereof further includes an algal Rubisco SSU protein or a modified higher plant Rubisco SSU protein.
7. The plant or part thereof of embodiment 6, wherein the Rubisco SSU protein is the algal Rubisco SSU protein, and wherein the one or more RBMs and the algal Rubisco SSU protein are from the same algal species.
8. The plant or part thereof of embodiment 6, wherein the Rubisco SSU protein is the modified higher plant Rubisco SSU protein, and wherein the modified higher plant Rubisco SSU includes one or more amino acid substitutions for an algal Rubisco SSU corresponding to residues 23, 24, 87, 90, 91, and 94 in SEQ ID NO: 60.
9. The plant or part thereof of embodiment 8, wherein: the amino acid substitution is at residue 23 and the substituted amino acid is Glu or Asp; the amino acid substitution is at residue 24 and the substituted amino acid is Glu or Asp; the amino acid substitution is at residue 87 and the substituted amino acid is Ala, Ile, Leu, Met, Phe, Trp, Tyr, or Val; the amino acid substitution is at residue 90 and the substituted amino acid is Ala, Ile, Leu, Met, Phe, Trp, Tyr, or Val; the amino acid substitution is at residue 91 and the substituted amino acid is Arg, His, or Lys; and/or the amino acid substitution is at residue 94 and the substituted amino acid is Ala, Ile, Leu, Met, Phe, Trp, Tyr, or Val.
10. The plant or part thereof of embodiment 4, wherein the heterologous polypeptide is the Rubisco LSU and the one or more RBMs are linked to the N-terminus or C-terminus of the Rubisco LSU, optionally through a linker polypeptide.
11. The plant or part thereof of embodiment 4, wherein the heterologous polypeptide is the membrane anchor and the membrane anchor anchors the heterologous polypeptide to a thylakoid membrane of a chloroplast and is optionally selected from the group consisting of a membrane bound protein, a protein that binds to a membrane-bound protein, a transmembrane domain, and a lipidated amino acid residue in the heterologous polypeptide.
12. The plant or part thereof of embodiment 4, wherein the heterologous polypeptide is the starch binding protein and the starch binding protein includes an alpha-amylase/glycogenase; a cyclomaltodextrin glucanotransferase; a protein phosphatase 2C 26; an alpha-1,4-glucanotransferase; a phosphoglucan, water dikinase; a glucan 1,4-alpha-glucosidase; or a LC19.
13. The plant or part thereof of any one of embodiments 1-12, wherein the chimeric polypeptide is localized to a chloroplast stroma of at least one chloroplast of a plant cell of the plant or part thereof, and wherein the plant cell is a photosynthetic cell.
14. The plant or part thereof of any one of embodiments 1-13, wherein the plant is a C3 crop plant selected from the group consisting of cowpea, soybean, cassava, rice, wheat, plantain, yam, sweet potato, and potato.
15. A genetically altered higher plant or part thereof, including: a polypeptide including two or more RBMs, and one or both of: an algal Rubisco-binding membrane protein (RBMP); and a Rubisco SSU protein.
16. The plant or part thereof of embodiment 15, wherein the polypeptide is a stabilized polypeptide that has been modified to remove one or more chloroplastic protease cleavage sites, and wherein the polypeptide optionally includes EPYC1 or CSP41A.
17. A method of producing the genetically altered plant of any one of embodiments 1-14, including: a) introducing a first nucleic acid sequence encoding the chimeric polypeptide including one or more RBMs and the heterologous polypeptide or the polypeptide including two or more RBMs, and optionally introducing a second nucleic acid sequence encoding the Rubisco SSU protein into a plant cell, tissue, or other explant; b) regenerating the plant cell, tissue, or other explant into a genetically altered plantlet; and c) growing the genetically altered plantlet into a genetically altered plant including the first nucleic acid sequence encoding the chimeric polypeptide including one or more RBMs and the heterologous polypeptide, and optionally, the second nucleic acid sequence.
18. A method of producing the genetically altered plant of embodiment 15, including: a) introducing a first nucleic acid sequence encoding a stabilized polypeptide including two or more RBMs, and introducing one or both of a second nucleic acid sequence encoding the algal RBMP and a third nucleic acid sequence encoding the Rubisco SSU protein into a plant cell, tissue, or other explant; b) regenerating the plant cell, tissue, or other explant into a genetically altered plantlet; and c) growing the genetically altered plantlet into a genetically altered plant including the first nucleic acid sequence encoding the stabilized polypeptide including two or more RBMs, and one or both of the second nucleic acid sequence encoding the algal Rubisco-binding membrane protein (RBMP) and the third nucleic acid sequence encoding the Rubisco SSU protein.
19. A chimeric polypeptide including one or more, two or more, or three or more Rubisco-binding motifs (RBMs) and a heterologous polypeptide, wherein the RBM includes the peptide sequence W[+]xxΨ[−] (SEQ ID NO: 28), SEQ ID NO: 27, or an amino acid sequence motif including WR or WK, where the W is assigned to position ‘0’, and which motif scores 5 or higher using the following criteria: points are assigned as follows: R or K in −6 to −8: +1 point; P in −3 or −2: +1 point; D/N at −1: +1 point; optionally D/E at +2 or +3: +1 point; A/I/LJV at +4: +2 points; and D/E/COO⁻ terminus at +5: +1 point.
20. A synthetic pyrenoid including at least one chimeric polypeptide described herein, wherein the synthetic pyrenoid is contained in a higher plant cell.
21. A genetically altered higher plant or part thereof, including: an algal Rubisco SSU protein, and at least one of the following: a stabilized polypeptide including two or more RBMs; a polypeptide containing part or all of an algal Rubisco-binding membrane protein (RBMP); or one or more RBMs fused to a heterologous polypeptide that localizes to a thylakoid membrane of a chloroplast, wherein the heterologous polypeptide that localizes to a thylakoid membrane of a chloroplast includes at least one of: a membrane bound protein, a protein that binds to a membrane-bound protein, a transmembrane domain, or a lipidated amino acid residue in the heterologous polypeptide.

Having generally described various embodiments of the invention, the same will be better understood by reference to certain specific examples, which are included herein to further illustrate the invention and are not intended to limit the scope of the invention as defined by the embodiments.

### EXAMPLES

The present disclosure is described in further detail in the following examples, which are not in any way intended to limit the scope of the disclosure as embodimented. The attached figures are meant to be considered as integral parts of the specification and description of the disclosure. The following examples are offered to illustrate, but not to limit the embodimented disclosure.

### Example 1

### Identification of Rubisco-Binding Motifs in EPYC1.

This example describes in vitro approaches used to identify and characterize Rubisco-binding motifs (RBMs) in EPYC1.

### Materials and Methods

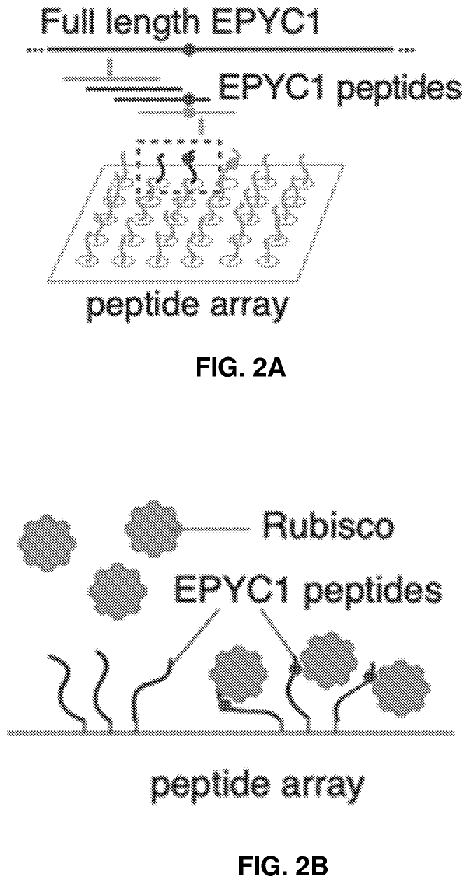

Peptide Tiling Arrays In order to understand the structural basis for EPYC1-Rubisco binding, the motif(s) of EPYC1 that bind to Rubisco needed to be identified. Circular dichroism suggested that purified EPYC1 was intrinsically disordered (Wunder et al., Nat. Commun. 9: 5076, 2018), which was consistent with predictions from the EPYC1 primary sequence (Mackinder et al., PNAS 113: 5958-5963, 2016). On the basis of this observation, and because of the short length of the EPYC1 primary sequence repeats, it was hypothesized that the RBMs of EPYC1 were short and could bind to Rubisco without a need for tertiary folds. Therefore, to identify EPYC1 regions that bind to Rubisco, peptide arrays consisting of 18, 22 or 25 amino acid peptides tiling across the full length EPYC1 sequence were synthesized (FIG. 2A), and probed with Rubisco (FIG. 2B).

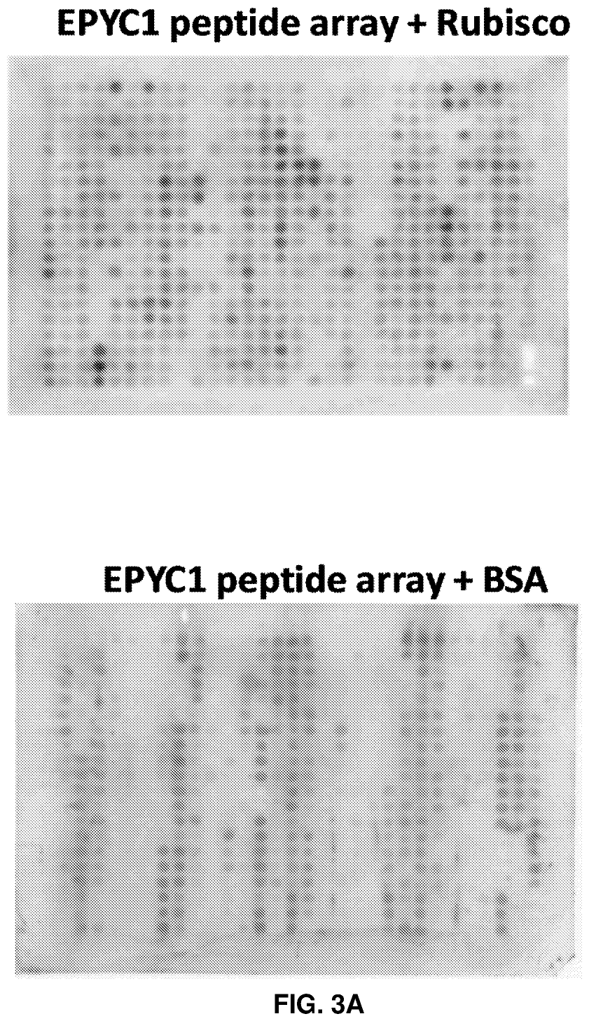

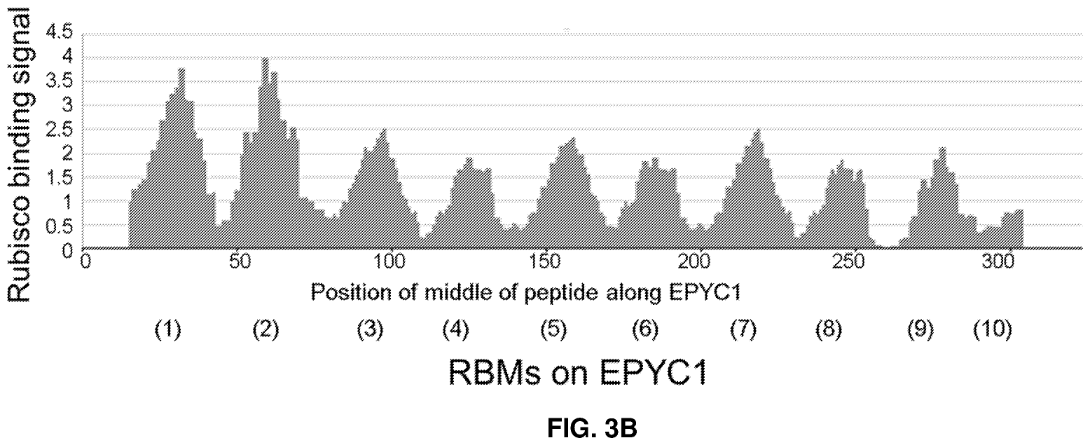

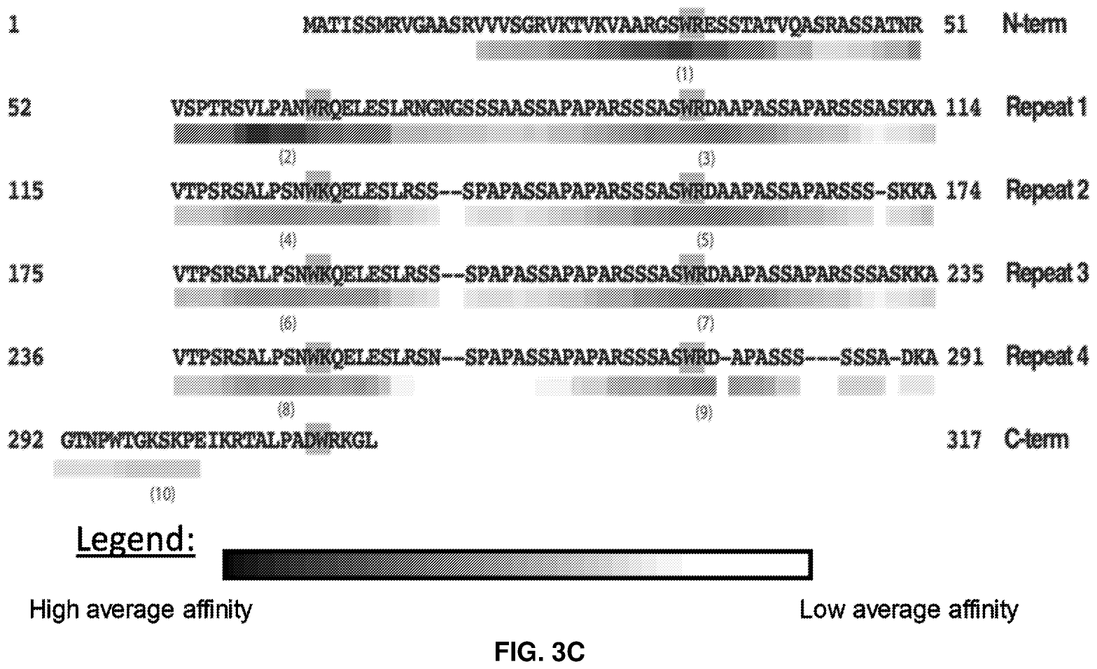

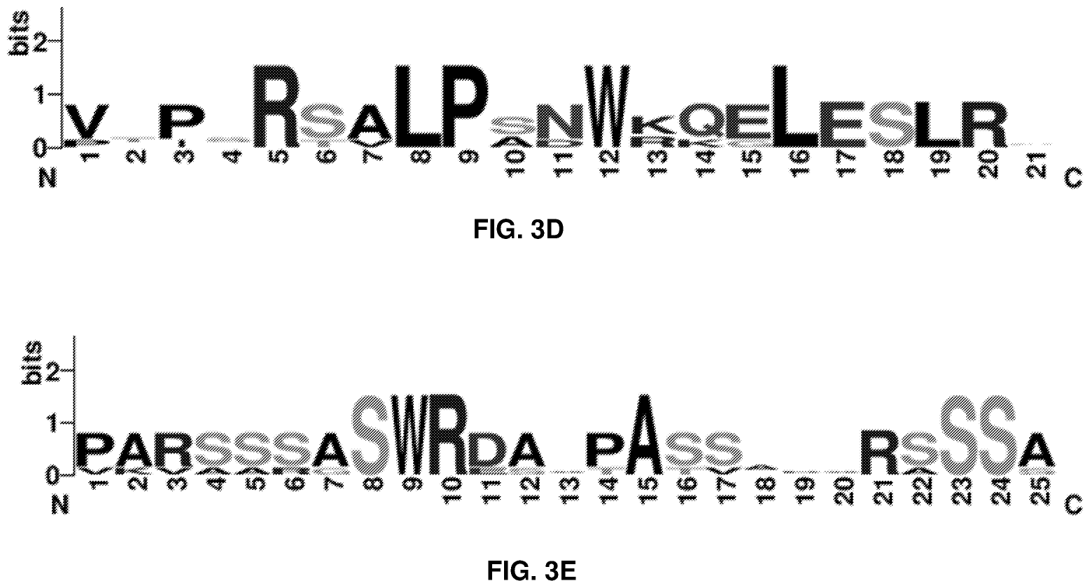

Peptide arrays were purchased from the MIT Biopolymers Laboratory. The tiling array was composed of 18-amino-acid peptides that tiled over the full length EPYC1 sequence with a 3 amino acid step size (FIG. 2A). In the substitution arrays, peptides were synthesized to systematically evaluate every possible one-amino acid substitution in RBM 2 on EPYC1. In each peptide, one of the amino acids was mutated to one of the other 19 amino acids. The arrays were activated by methanol, then incubated in Binding Buffer (50 mM HEPES, 50 mM KOAc, 2 mM Mg(OAc)₂, 1 mM CaCl₂, 200 mM sorbitol) for 3×10 min washes. The arrays were then incubated for at 4° C. with 1mg Rubisco overnight (FIG. 2B). The arrays were washed again in Binding Buffer to remove any unbound Rubisco. Using a semi-dry transfer apparatus, bound Rubisco was transferred to a PVDF membrane and detected with Rubisco antibody (FIG. 3A). Spots with higher binding affinity to Rubisco resulted in stronger signals (FIG. 3B). Bovine serum albumin was used as a negative control to confirm the specificity of binding between the peptide array and Rubisco. Incubation with bovine serum albumin produced a different binding pattern (FIG. 3A).

Surface Plasmon Resonance (SPR) Assay Rubisco was immobilized on a surface and peptides in solution were flowed over the surface. Surface plasmon measurements of binding of individual peptides to Rubisco were assayed (FIG. 3B).

### Results

EPYC1 Contains Ten RBMs The peptide tiling arrays and SPR assays revealed multiple RBMs on EPYC1 (FIGS. 3B-3C). The RBMs were specific to Rubisco, as incubation with bovine serum albumin instead of Rubisco produced a different binding pattern. The observation that short peptides from EPYC1 were able to bind to Rubisco confirmed that EPYC1 RBMs could bind Rubisco in the absence of tertiary folds. Further, it was observed that multiple RBMs along the EPYC1 sequence were able to bind Rubisco (FIGS. 3B-3C). This observation indicated that EPYC1 acted as a “linker”, and would be able to bind several different Rubisco holoenzymes to aggregate them.

In particular, ten RBMs were identified on EPYC1 (FIGS. 3B-3C), suggesting that an EPYC1 protein can bind up to ten Rubisco holoenzymes. This finding was in contrast to previous publications, which had suggested four (Mackinder et al., PNAS 113: 5958-5963, 2016) or five (Wunder et al., Traffic 20(6):380-389, 2019) RBMs on EPYC1. In fact, these results indicated that each of the four previously defined repeats contained two RBMs (FIGS. 3B-3C), and that there were two further RBMs, one at each terminus of the EPYC1 protein. The ten RBMs identified were spaced evenly across the protein, with approximately 30 amino acids between binding peaks. Analysis of the sequences of the RBMs revealed that the ten RBMs shared sequence homology. The homology was strongest among alternating RBMs, referred to as “even” RBMs (RBMs 2, 4, 6, 8, and 10) and “odd” RBMs (RBMs 1, 3, 5, 7, and 9). The even RBMs 2, 4, 6, and 8 shared a sequence V(S/T)P(S/T)RS(A/V)LP(A/S)NW(R/K)QELESLR (SEQ ID NO: 45), and even RBM 10 shared a portion of this sequence: RTALPADWRKGL (SEQ ID NO: 67). FIG. 3D illustrates the consensus sequence for the even RBMs on EPYC1. The odd RBMs 3, 5, 7, and 9 shared a sequence PARSSSASWRD(A)APASS(APAR) (SEQ ID NO: 46). Odd RBM 1 was the most different from the other odd RBMs, but it shared the central sequence SWR and identical or similar amino acids at 4 other positions. FIG. 3E illustrates the consensus sequence for the odd RBMs on EPYC1. Importantly, all ten even and odd RBMs shared a central WR/K sequence (FIGS. 3D-3E). This shared central sequence, and the homology between the RBMs, indicated that the RBMs bound to Rubisco using a common mechanism.

The results from the SPR assay indicated that the Kd for each RBM was in the range of 3 mM.

### Conclusions

Whereas previous publications had suggested four (Mackinder et al., PNAS 113: 5958-5963, 2016) or five (Wunder et al., Traffic 20(6):380-389, 2019) RBMs on EPYC1, the results presented above surprisingly identified ten RBMs. This higher number of RBMs would be favorable for the phase separation observed in pyrenoids, as higher valencies of binding sites have previously been shown to promote phase separation (Li et al., Nature 483(7389):336-40, 2012).

The regular distance between RBMs on EPYC1 (approximately 30 amino acids between binding peaks) was hypothesized to be an indication of selective pressure for an optimal distance between RBMs. Placing binding sites too close together could prevent efficient interaction with multiple Rubiscos, whereas placing the binding sites too far apart could produce a matrix where Rubisco was not sufficiently dense for optimal CO₂ concentration.

Finally, the low affinity of individual RBMs on EPYC1 could explain how the pyrenoid matrix is able to mix internally on the timescale of seconds in spite of the high valency of both Rubisco and EPYC1. In this scenario, the high valency of RBMs on EPYC1 would compensate for their low individual affinities, leading to a high overall avidity that keeps the pyrenoid matrix together. Indeed, multivalent weak interactions have been identified as a hallmark of phase-separated organelles (Li et al., Nature 483(7389):336-40, 2012).

### Example 2

### Characterization of the Rubisco-EPYC1 Interaction and Identification of Critical Residues

This example describes the characterization of the Rubisco-EPYC1 interaction using a cryoelectron microscopy (cryoEM) structure of Rubisco bound to a fragment of EPYC1. In addition, the example describes in vitro and in vivo approaches that identified critical residues on EPYC1 and on Rubisco for the interaction between EPYC1 and Rubisco.

### Materials and Methods

Strains and culture conditions: Chlamydomonas reinhardtii strain cMJ030 wild-type (WT) was maintained in the dark or low light (-10 pmol photons m⁻² s⁻¹) on 1.5% agar plates containing TAP with revised trace elements (Kropat et al., Plant J. 66: 770-780, 2011). For Rubisco extraction, a loopful of cells was inoculated into 500 mL TAP medium in a 1L flask and grown to ˜4×10⁶ cells/mL at room temperature, 100 μmol photons m⁻² s⁻¹, at 3% CO₂, shaking at 200 rpm.

### Protein Extraction

Rubisco was purified from Chlamydomonas reinhardtii strain cMJ030. Cells were disrupted by ultrasonication in lysis buffer (10 mM MgCl₂, 50 mM Bicine, 10 mM NaHCO₃, 1 mM dithiothreitol (DTT) pH 8.0) supplemented with Halt Protease Inhibitor Cocktail, EDTA-Free (Fisher Scientific). The soluble lysate was fractionated by ultracentrifuge in a 10%-30% sucrose gradient in a SW 41 Ti rotor at 35,000 rpm for 20 hours at 4° C. Rubisco-containing fractions were applied to an anion exchange column (MONO Q 5/50 GL, GE Healthcare) and fractionated by using a linear salt gradient from 0 to 0.5 M NaCl (10 mM MgCl₂, 50 mM Bicine, 10 mM NaHCO₃, 1 mM dithiothreitol pH 8.0).

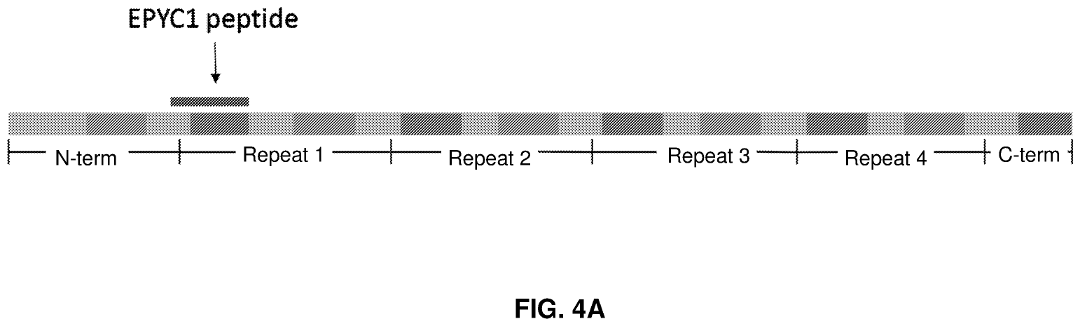

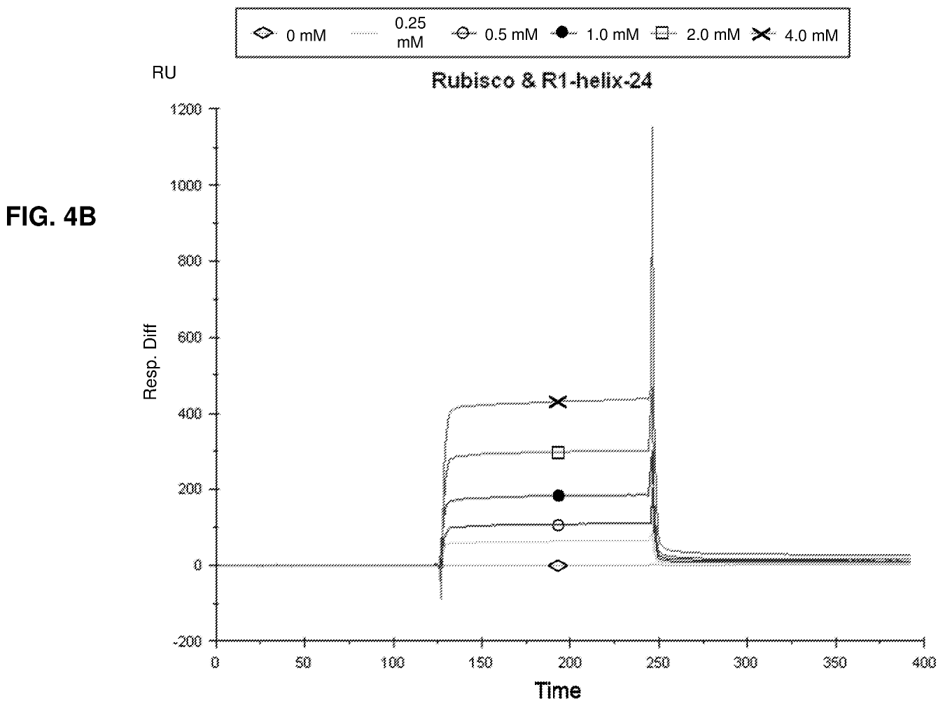

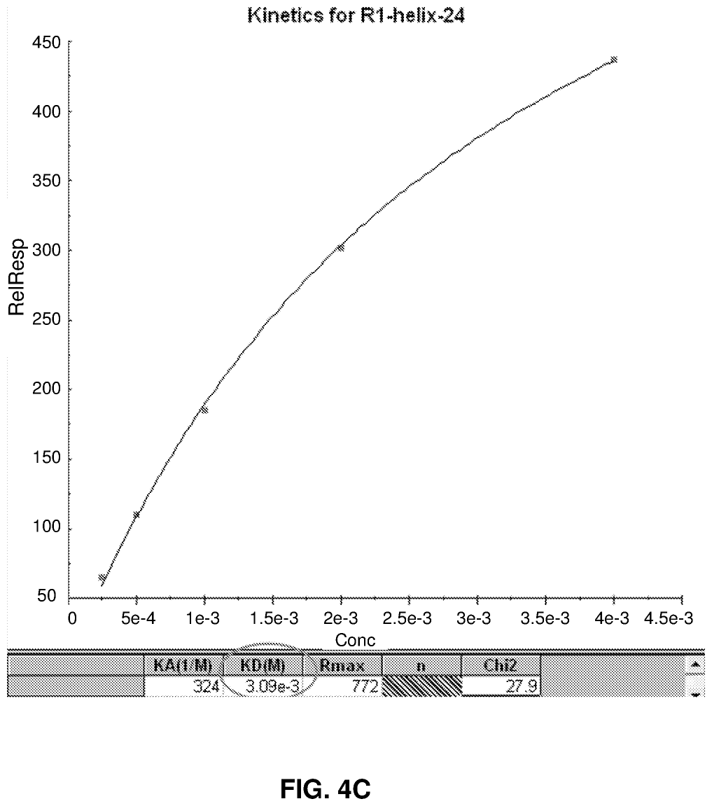

Cryoelectron Microscopy: Single particle cryoelectron microscopy on Rubisco bound to a peptide fragment of EPYC1 was performed. A peptide fragment of EPYC1 representing a single RBM (FIG. 4A) was used rather than the entire EPYC1 protein because mixing complete EPYC1 with Rubisco has been shown to lead to phase separation (Wunder et al., Nat. Commun. 9(1):5076, 2018). This would have interfered with identification of single Rubisco particles for classification and structural analysis. The EPYC1 fragment used in these experiments corresponded to RBM 2 of EPYC1 (FIG. 4A). RBM 2 was chosen because this 24 amino acid fragment had the highest binding affinity (Kd=3 mM) of all peptides tested (FIGS. 4B-4C).

The low Rubisco-binding affinity of individual EPYC1 RBMs meant that millimolar concentrations of peptide were needed to approach full occupancy of Rubisco (FIG. 5A). This led to challenges including peptide insolubility and high background signal in the electron micrographs. Despite these challenges, a 2.8 Å structure of Rubisco bound to the 24 amino acid EPYC1 fragment was obtained.

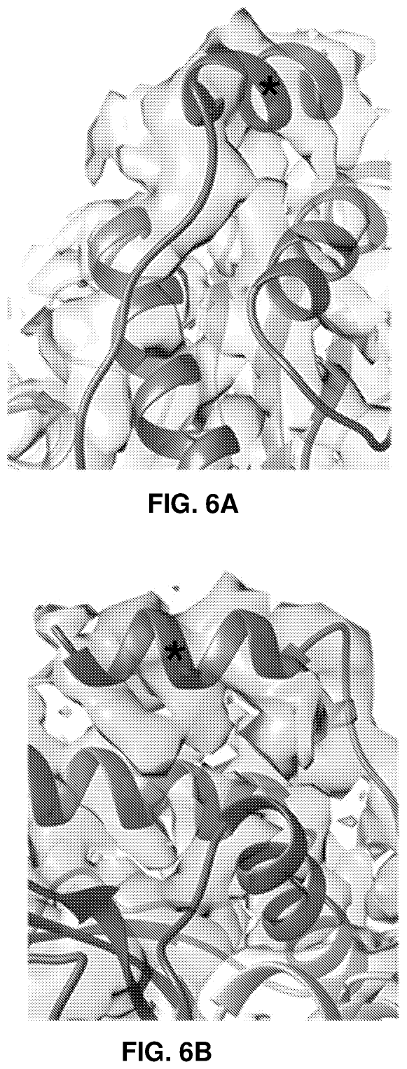

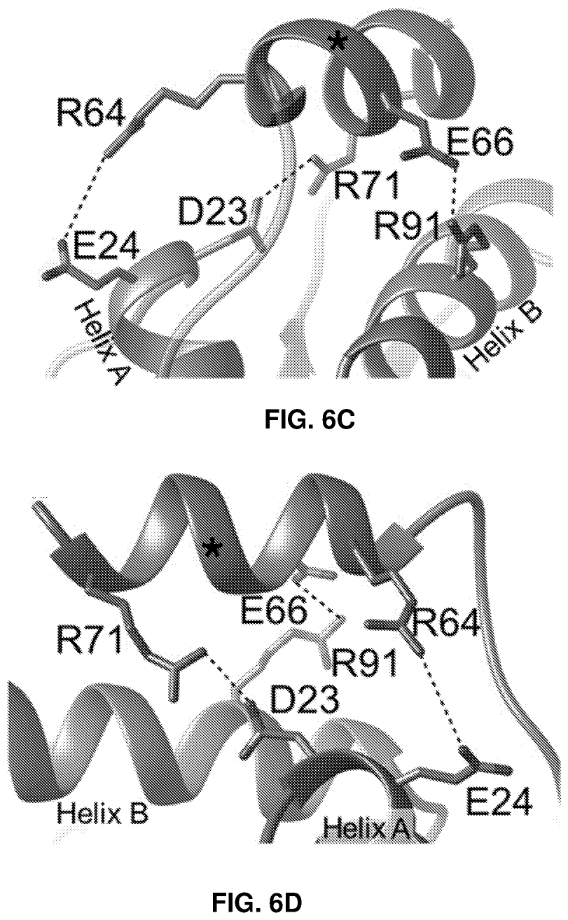

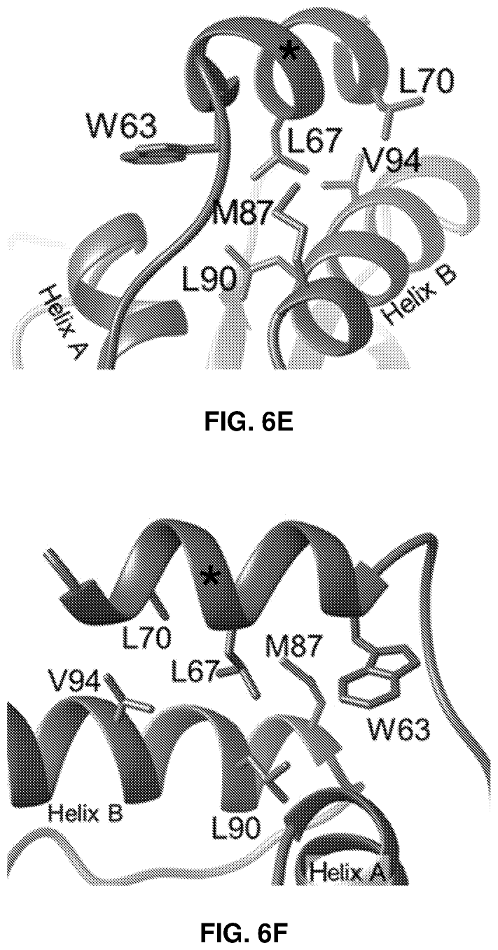

Atomic Modeling: A full model for C. reinhardtii Rubisco was produced from an X-ray structure (PDB entry 1GK8; Taylor et al., J. Biol. Chem. 276: 48159-48164, 2001) and used for rigid body fitting to a local resolution filtered cryo-EM map with an average resolution of 2.8 Å using UCSF Chimera (Pettersen et al., J. Comput. Chem. 25: 1605-1612, 2004). After rigid body fitting of the full complex, initial flexible fitting was performed in COOT (Emsley et al., Acta. Crystallogr. D. Biol. Crystallogr. 66: 486-501, 2010) by manually going through the entire peptide chain of a single large and small Rubisco subunit before applying the changes to the other seven large and seven small subunits. The sequence of the peptide was used to predict secondary structure elements using JPred4 (Drozdetskiy et al., Nucl. Acids Res. 43: W1, W389-W394, 2015), which resulted in the prediction of the C-terminal region (NWRQELES; SEQ ID NO: 86) to be α-helical. With that knowledge, the peptide was built manually into the density using COOT. 3D structure predictions results did not fit the density well. After a rough fit using COOT, additional real space refinement of the entire complex was performed using Phenix (Adams et al., Acta. Crystallogr. D. Biol. Crystallogr. 66: 213-221, 2010). Models were subjected to an all-atom structure validation using MolProbity (Chen et al., Acta. Crystallogr. D. Biol. Crystallogr. 66: 12-21, 2010). FIGS. 5A-5E and 6A-6F were produced using UCSF Chimera.

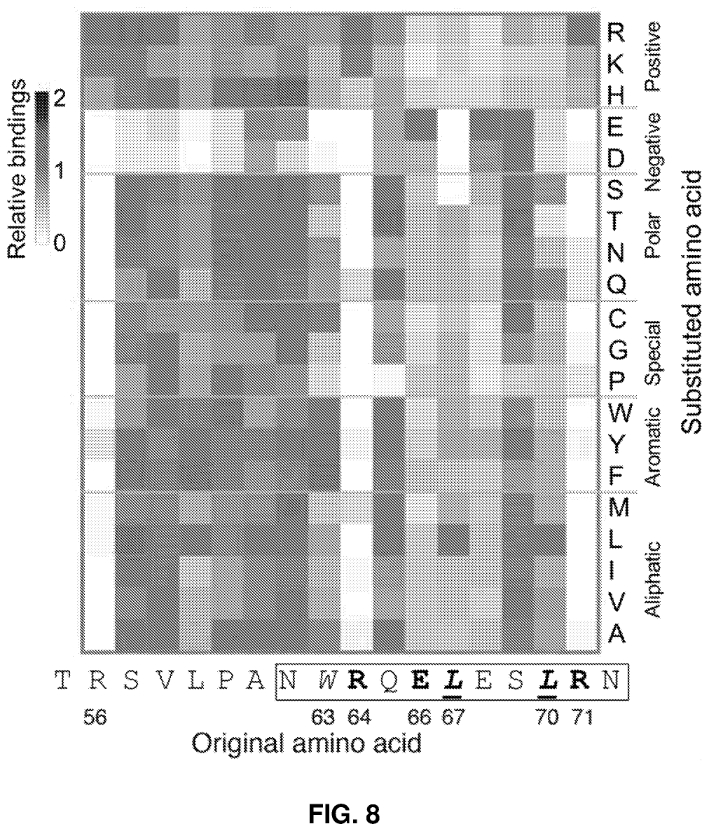

Mutagenesis of EPYC1, Mutagenesis of Rubisco, Yeast Two-hybrid, Peptide Arrays and SPR Assays To determine the importance of individual EPYC1 residues for binding to Rubisco, the impact on Rubisco binding of every possible single amino acid substitution for EPYC1 RBM 2 was determined (FIG. 8). To determine the importance of individual Rubisco residues for binding to EPYC1, targeted mutations of the amino acids identified from atomic modeling were tested in a yeast two-hybrid assay as in van Nues and Beggs (Genetics 157: 1451-1467, 2000). Peptide arrays and SPR assays were performed as in Example 1.

### Results

Structural Characterization Showed That Rubisco Bound Eight EPYC1 Fragments: The Rubisco holoenzyme consists of eight large subunits and eight small subunits, which come together to form an L8S8 holoenzyme. The eight large subunits (LSUs) form the core of the holoenzyme, and four small subunits (SSUs) “cap” each end of this core. Analysis of the 2.8 A structure of Rubisco bound to the 24 amino acid EPYC1 fragment revealed that the EPYC1 peptides were clearly visible and bound to the Rubisco small subunits (FIGS. 5B-5E). Each Rubisco holoenzyme was shown to bind up to eight EPYC1 molecules. This structural result further supported a model where EPYC1 and Rubisco formed a multivalent network.

The observation that EPYC1 bound to the Rubisco SSUs was consistent with the assembly mechanism of Rubisco. During Rubisco holoenzyme biogenesis, the eight LSUs first assemble together into an intermediate complex, and then eight SSUs are added to the complex. If EPYC1 bound to the large subunit, the intermediate complex could be recruited into the pyrenoid. In contrast, EPYC1′s interaction with the Rubisco SSU ensures that Rubisco is not recruited into the pyrenoid until it is fully assembled. Further, unassembled Rubisco small subunits likely do not have sufficient valency on their own to be recruited into the Rubisco matrix in the pyrenoid.

Comparison of the electron density map of the 2.8 Å structure of Rubisco bound to the 24 amino acid EPYC1 fragment with a published X-ray structure revealed important differences.

Characterization of the Rubisco-EPYC1 Interaction Mechanism: As shown in FIGS. 6A-6B, the EPYC1 peptide (in red) formed an extended chain, a portion of which formed an alpha example helix that sat on top of the Rubisco SSU's two α-helices (in blue). The location of the peptide binding site on Rubisco was consistent with a previous study, which found that mutations in these a-helices disrupted Rubisco's assembly into a pyrenoid in vivo (Meyer et al., PNAS 109(47):19474-9, 2012). The C-terminal region of the EPYC1 peptide (NWRQELESLRN; SEQ ID NO: 113) was well resolved and formed a helix that ran parallel to helix B of the Rubisco small subunit. The N-terminus of the EPYC1 peptide extended the trajectory of the helix and followed the surface of the Rubisco SSU. The side chains of the N-terminus could not be resolved, suggesting that this region was more conformationally flexible.

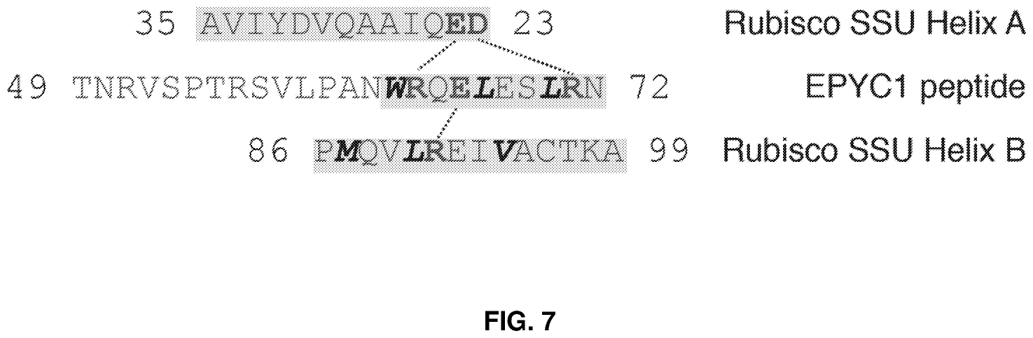

To gain insights into the mechanism of binding, an atomic model was fit to the electron density map. The atomic model suggested that binding between EPYC1 and Rubisco was mediated by salt bridges (FIGS. 6C-6D) and a hydrophobic pocket (FIGS. 6E-6F). As shown in FIGS. 6C-6D, three prominent residue pairs likely formed salt bridges. These residue pairs were EPYC1 residues R64 and R71, which interacted with E24 and D23 of Rubisco SSU a-helix A, respectively, and EPYC1 residue E66, which interacted with R91 of Rubisco small subunit a-helix B (FIG. 7). In addition, as shown FIGS. 6E-6F, a hydrophobic pocket was formed by L67 of EPYC1 and M87, L90 and V94 of Rubisco small subunit helix B (FIG. 7).

Biochemical Methods Confirmed Critical Residues in RBM 2 of EPYC1 for the EPYC1-Rubisco Interaction: As shown in FIG. 8, the results obtained from mutating individual amino acids supported the structural model for the EPYC1-Rubisco interaction. Notably, the three arginines of the EPYC1 peptide, R56, R64 and R71, appeared to be most critical for binding to Rubisco, as substitution of any of those residues with almost any other amino acid eliminated binding. The requirement for an arginine or lysine at R64 and R71 was explained by their interactions with Rubisco SSU E24 and D23, respectively. While R56 was not well resolved in the structure, it was thought that it may interact with the backbone oxygen of E433 of the Rubisco LSU.

In addition, the residues W63, L67, and L70 of the EPYC1 fragment also appeared to be important, as most substitutions decreased binding (FIG. 8). These results were consistent with the structural results, as W63, L67, and L70 contributed to the hydrophobic pocket. Further, E66 and E68 of the EPYC1 fragment appeared to be important. The importance of E66 was explained by the structure, where this residue interacted with R91 of Rubisco SSU alpha helix B. The EPYC1 region between R56 and W63 exhibited few sequence requirements other than a preference against negatively charged residues, which was likely due to the proximity of negatively charged Rubisco SSU residues E24 and D31.

Surface plasmon resonance (SPR) experiments supported the results discussed above. In the SPR assays, each residue in the EPYC1 peptide was mutated to alanine individually. The results showed that mutation of R56, W63, R64, L67, L70, or R71 led to a decrease in binding affinity to the Rubisco SSU. Significantly, the importance of R64, L67, and R71 was consistent with the cryoelectron microscopy results discussed above. Mutation of N62 or Q65 did not significantly alter the binding affinity of EPYC1 for the Rubisco SSU.

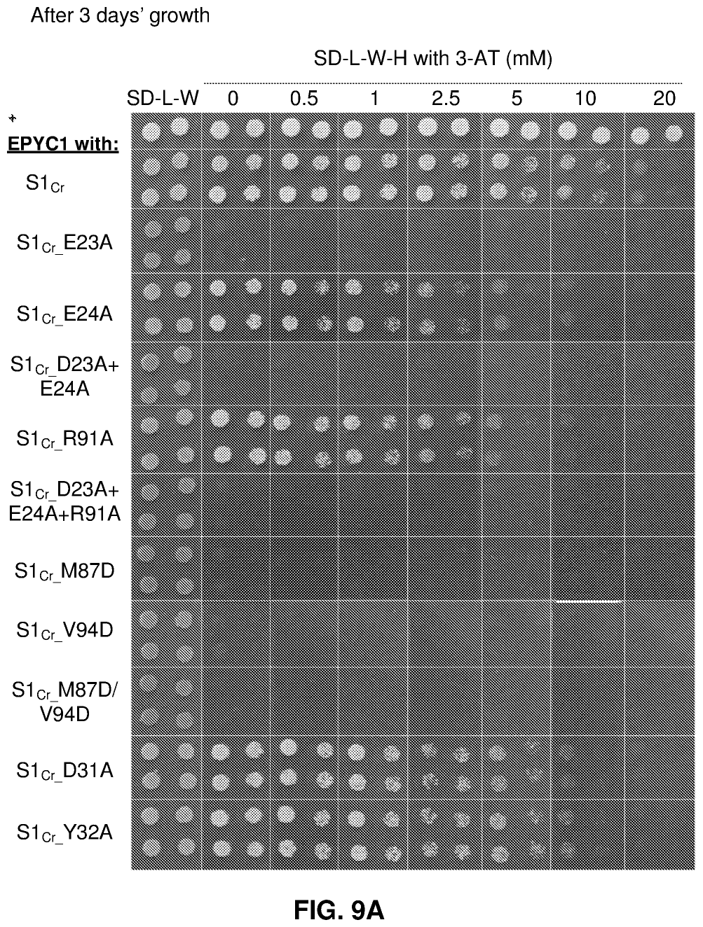

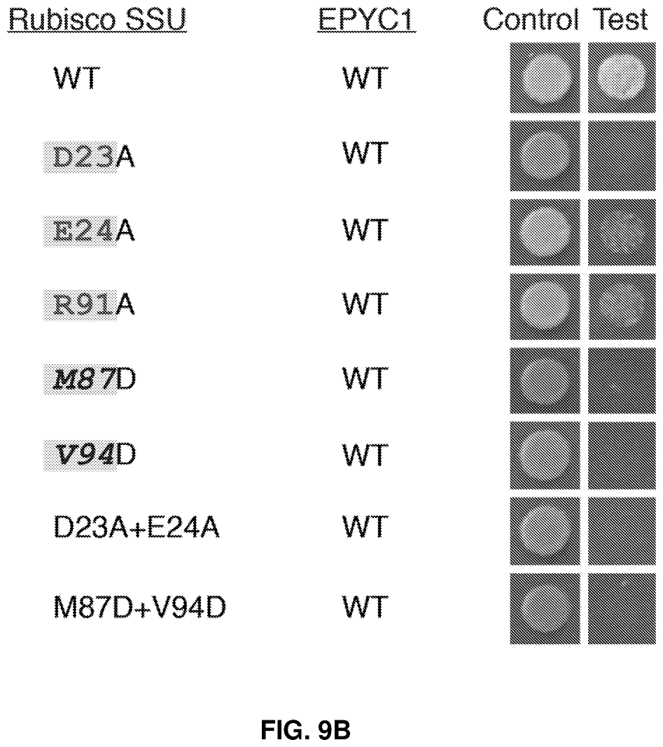

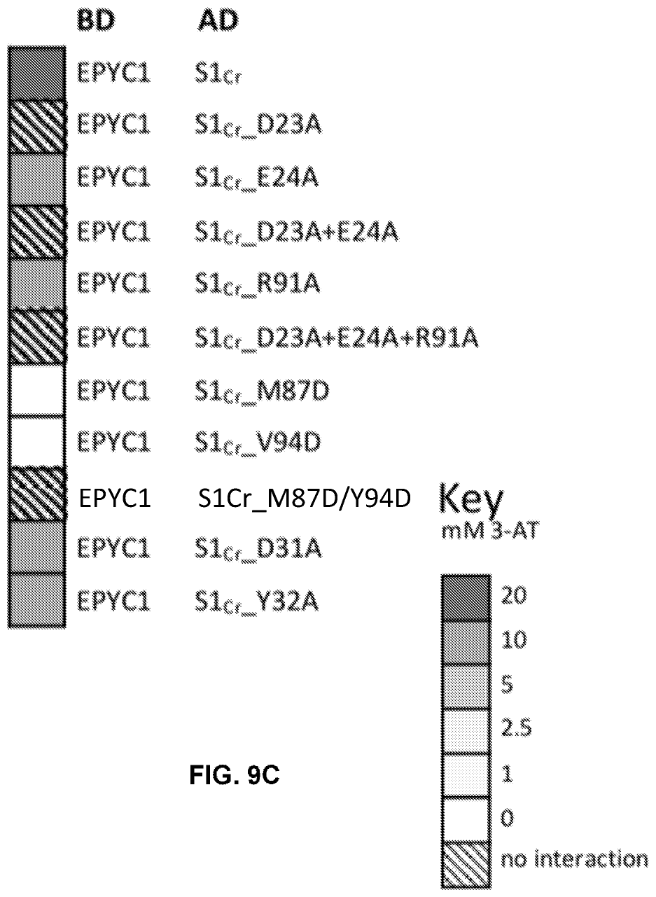

Confirmation of Critical Rubisco Residues for the EPYC1-Rubisco Interaction: To validate the importance of Rubisco residues for binding to EPYC1, the impact of mutations in critical Rubisco SSU residues on interactions between EPYC1 and the Rubisco SSU was determined in a yeast two-hybrid assay. As shown in FIGS. 9A-9C, the mutation D23A had a severe impact on the Rubisco SSU-EPYC1 interaction, which was expected from the contribution of this residue to a salt bridge with R71 of EPYC1. In addition, the mutations E24A and R91A each showed a moderate defect in binding between Rubisco SSU and EPYC1, consistent with their contributions to salt bridges with R64 and E66 of EPYC1, respectively. Further, the mutations M87D and V94D each had a severe impact on the Rubisco SSU-EPYC1 interaction, as was expected from their participation in the hydrophobic pocket. Combinations of these mutations abolished the interactions completely.

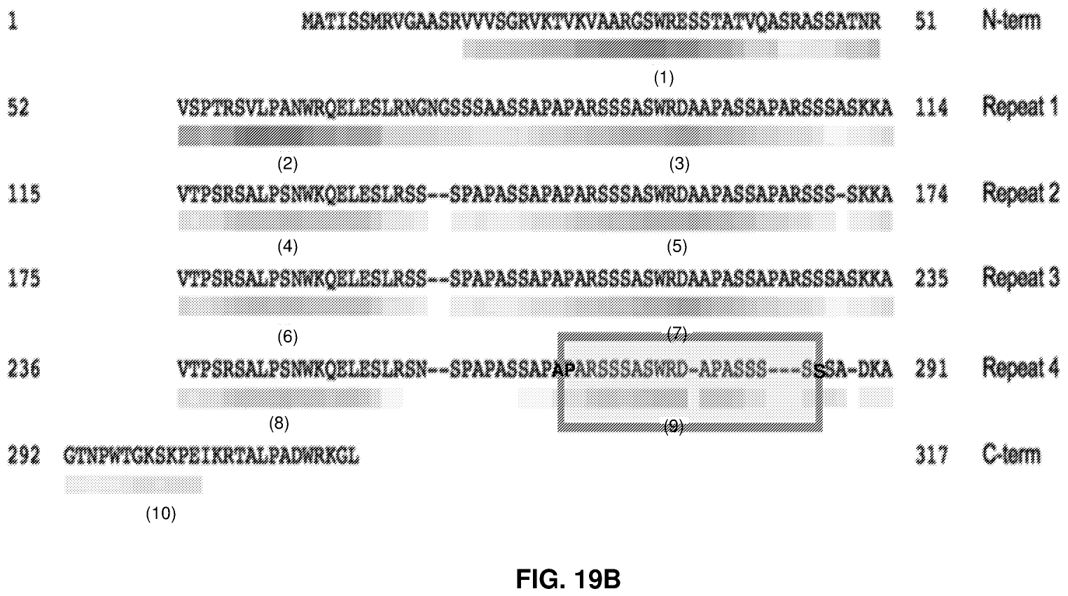

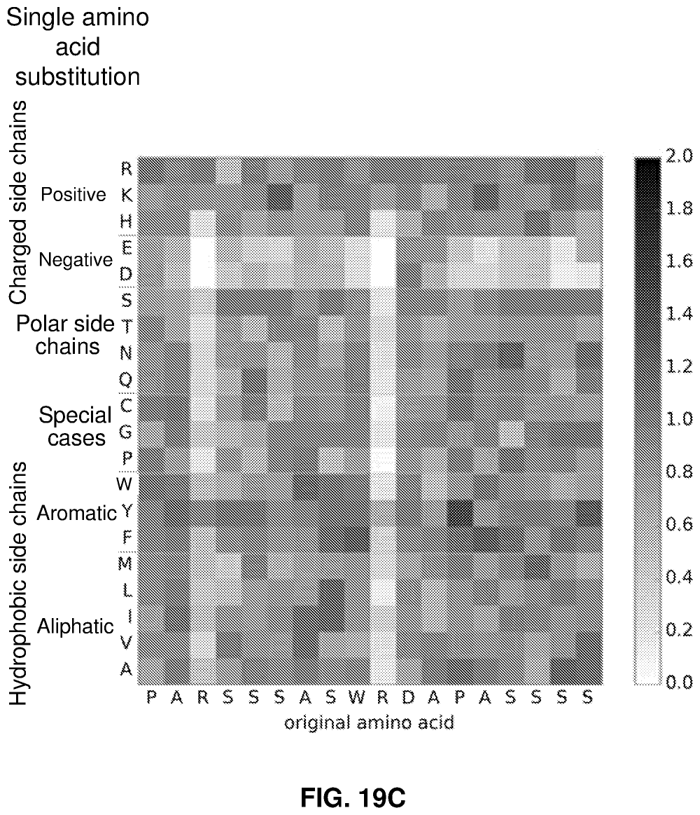

Even and Odd RBMs on EPYC1 Bind the Same Site on Rubisco As described in Example 1, the near-identity of the sequences of all the even RBMs on EPYC1 (2, 4, 6, 8) strongly suggested that all of these RBMs bound to Rubisco in the same way. To determine whether the odd RBMs bound to the same site on Rubisco as the even RBMs, the impact of every single amino acid substitution in RBM 9 on binding to Rubisco was systematically tested. The results shown in FIGS. 19A-19C revealed a pattern similar to that observed with RBM 2, with two arginines that were found 7 residues apart in the RBM 9 amino acid sequence proving to be very important for binding to Rubisco. Additionally, negative charges on RBM 9, which were found in similar locations as in RBM 2, disrupted binding to Rubisco. The amino acid substitution array shown in FIG. 19C confirmed the importance of the charged residues in RBM 9.

One notable difference between RBM 9 and RBM 2 was that most mutations after the WR in RBM 9 did not disrupt binding. This difference may be due to the observation that RBM 2 formed an alpha helix, whereas RBM 1, RBM 3, RBM 5, RBM 7, and RBM 9 were predicted to be disordered. The similarity of the mutational sensitivity pattern between RBM 2 and RBM 9 suggested that all RBMs of EPYC1 bound to the same site on Rubisco.

Overall, the data presented in this example demonstrated that EPYC1 RBM 2 bound to the Rubisco small subunit alpha helices via specific salt bridge interactions and a hydrophobic pocket. Further, the results indicated that all RBMs of EPYC1 bound to the same site on Rubisco, as similar results were obtained with RBM 2 and RBM 9.

### Example 3

### RBMs on EPYC1 are Required for Phase Separation with Rubisco

This example describes in vitro phase separation experiments using EPYC1 mutants that showed RBMs of EPYC1 were required for phase separation of EPYC1 with Rubisco. In addition, this example provides a model for EPYC1-mediated formation of the Rubisco matrix in the pyrenoid.

### Materials and Methods

Mutagenesis of EPYC1: The central W and R/K of each RBM were mutated because those residues were present in all RBMs and their mutation disrupted binding in SPR and peptide array experiments (FIG. 10A).

In Vitro Phase Separation Assays: To determine the importance of the EPYC1 RBMs for pyrenoid Rubisco matrix formation, the impact of mutations in the RBMs on formation of phase separated EPYC1-Rubisco droplets was assayed in low (50 mM NaCl) and high (150 mM NaCl) salt concentrations. Liquid-liquid phase separation assays were performed as described in Wunder et al., Nat. Commun. 9: 5076, 2018.

### Results

RBMs are Required for Phase Separation of EPYC1 and Rubisco As shown in FIG. 10B, mutation of the central W in each RBM to alanine (A) completely abolished phase separation. In addition, mutation of the central K or R in RBM to A disrupted phase separation, and this effect was much more pronounced at the higher salt concentration of 150 mM NaCl. Importantly, mutating the WK or WR in either even or odd motifs alone disrupted phase separation, supporting the idea that both even and odd motifs contribute to Rubisco binding (FIG. 10B)

Overall, these results showed that the RBMs on EPYC1 were required for EPYC1-Rubisco phase separation in vitro.

### Example 4

### RBMs are Present in pyrenoid-Associated Proteins

This example describes proteomics and biochemical methods that revealed the presence of RBMs on pyrenoid-associated proteins.

### Materials and Methods

Electron Microscopy Cells were fixed and embedded in a low viscosity epoxy resin as described in Mackinder et al. (PNAS 113: 5958-5963, 2015; doi: 10.1073/pnas.1522866113). Thin sectioning was performed by the Core Imaging Lab, Department of Pathology, Rutgers University, and imaging was performed at the Imaging and Analysis Center, Princeton University, on a Philips CM100 FEG with an electron beam intensity of 100 keV.

Immunoprecipitation and Mass Spectrometry: A protein immunoprecipitation (IP) experiment was carried out using a polyclonal anti-RBM antibody in C. reinhardtii homogenates. The immunoprecipitate was analyzed by mass spectrometry (IP-MS). The immunopurification protocol, described in Mackinder et al. (Mackinder et al., PNAS 113: 5958-5963, 2015), was amended as follows. An anti-RBM antibody (YenZym Antibodies, South San Francisco) was immobilized on magnetic beads, in place of an anti-FLAG M2 antibody. Bound proteins were released and denatured in 1× Laemmli buffer with 50 mM beta-mercaptoethanol at 70° C. for 10 minutes. Samples were run on 10% SDS-PAGE gels, then Coomassie stained, and sectioned into four fragments of equal length, prior to protein digestion and mass spectrometric analysis.

Immunoblotting: To identify proteins that bound directly to the anti-RBM antibody, a Western blot was performed on SDS-PAGE separated total cell homogenates using the anti-RBM antibody. Total proteins were extracted, normalized to chlorophyll, separated by SDS-PAGE and western blotted as described in Heinnickel et al. (J. Biol. Chem. 288: 7024-7036, 2013). The primary anti-RBM antibody was used at a 1:7,500 concentration and the secondary horseradish-peroxidase conjugated goat anti-rabbit (Life Technologies) at a 1:15,000 concentration. To ensure even loading, technical replicated of the gels were stained with Coomassie.

Protein Sequence Alignment: Protein sequences were aligned with Clustal Omega (Sievers et al., Mol. Sys. Biol. 7: 539, 2011).

SPR Assays: The Rubisco-binding capacity for the C-terminal motif variant (W[+]xxΨ ) was determined using SPR by probing purified Rubisco with fifteen amino acid-long synthetic peptides. SPR assays were performed as in Example 1.

### Results

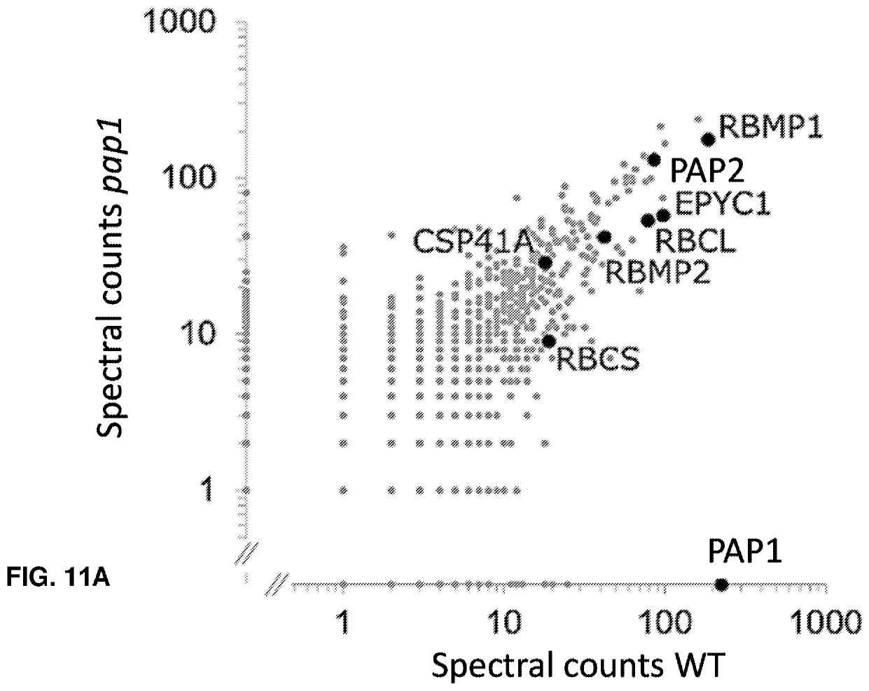

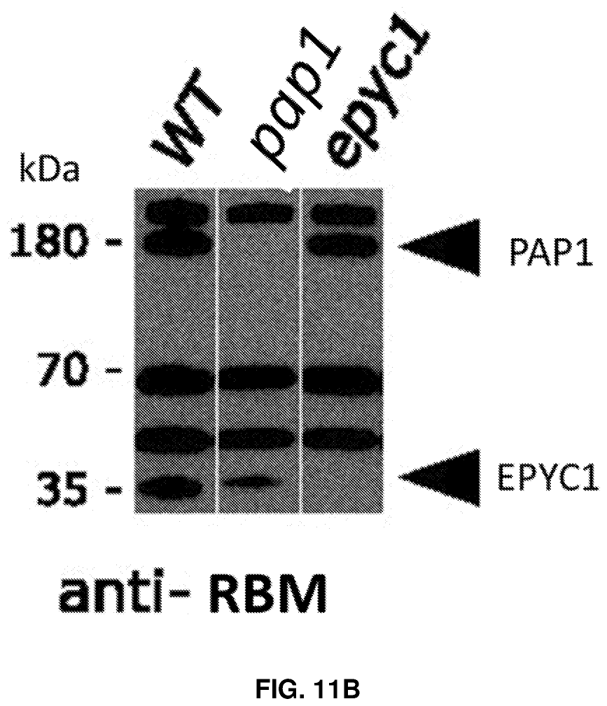

An Anti-RBM Antibody Binds Pyrenoid Proteins: The analysis of the IP experiment revealed that the anti-RBM antibody immunoprecipitated Rubisco as well as Rubisco-interacting proteins. These Rubisco-interacting proteins were EPYC1 and four previously uncharacterized proteins (Pyrenoid Associated Protein 1 (PAP1), PAP2, Rubisco-Binding Membrane Protein 1 (RBMP1), RBMP2, and CSP41A) (FIG. 11A). Immunoblotting consistently resolved five polypeptides (FIG. 11B). Polypeptides corresponding to the Rubisco large and small subunits were never detected. Strikingly, there was a remarkable agreement between the polypeptides observed in the Western blot and the predicted size of four of the five top anti-RBM antibody interactors identified by IP-MS. These proteins were not only present in the Rubisco interactome (Table S5 of Mackinder et al., Cell 171: 133-147, 2017), but they had also been identified as likely pyrenoid proteins in a recent proteome study of this organelle in C. reinhardtii (Table S1 of Zhan et al., PloS One 13: e0185039, 2018).

As shown in FIG. 11B, EPYC1 was conclusively identified by the absence of a matching polypeptide when performing an anti-RBM immunoblot on homogenates from a mutant lacking EPYC1 (epyc1). Similarly, PAP1 was conclusively identified by the absence of a matching polypeptide when performing an anti-RBM immunoblot on homogenates from a mutant lacking PAP1 (pap1).

Proteomic analysis by bins of the extract of the IP identified the following proteins (listed in order of increasing size). EPYC1 was identified as an approximately 35 kDa protein. As noted above, EPYC1 was conclusively identified by the absence of a matching polypeptide when performing an anti-RBM immunoblot on homogenates from epyc1. CSP41A, a chloroplast NAD-dependent epimerase, was identified as an approximately 45 kDa protein. An approximately 70 kDa protein with high homology to a Ca²⁺-binding anion channel of the bestrophin family was identified. This protein was previously uncharacterized, and so was named Rubisco-Binding Membrane Protein 1 (RBMP1). An approximately 166 kDa protein with three predicted transmembrane domains but no functional annotations was identified. The protein was also previously uncharacterized, and so was named Rubisco-Binding Membrane Protein 2 (RBMP2). No polypeptide matching the size of RBMP2 was observed in anti-RBM antibody immunoblots. PAP1 was identified as an approximately 180 kDa protein. As noted above, PAP1 was conclusively identified by the absence of a matching polypeptide when performing an anti-PAP1 immunoblot on homogenates from pap1 mutants. PAP2 was identified as an approximately 190 kDa protein with two predicted starch binding domains. The protein was previously uncharacterized but was identified as a PAP1 homolog, and was therefore named PAP2.

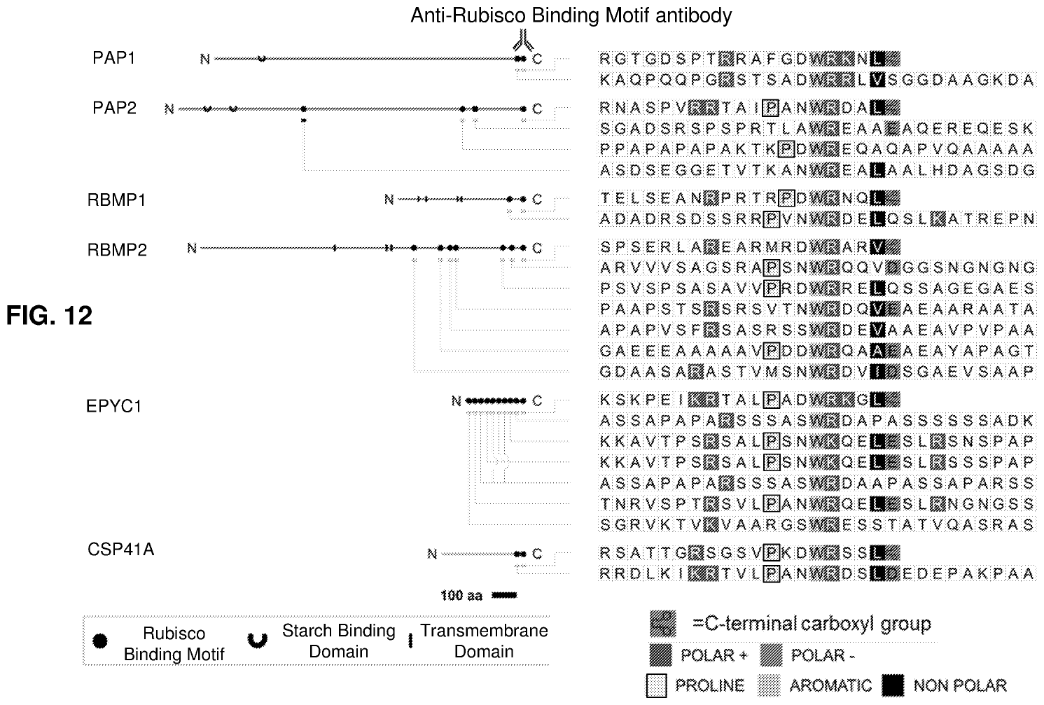

All Anti-RBM Antibody-Precipitated Proteins Share a Multivalent W[+]xxΨ[−] Motif The above results suggested that the six proteins shared the same epitope, allowing all of the proteins to be recognized and immunoprecipitated by the anti-RBM antibody. To identify shared epitopes on all proteins that might be recognized by the anti-RBM antibody, the peptide used to generate the anti-RBM antibody was aligned with the full-length sequence of all six pyrenoid proteins (FIG. 12). This peptide corresponded to the last nineteen residues of anti-RBM.

Significantly, the sequences of all six proteins ended with W[+]xxΨ, immediately followed by the stop codon (FIG. 12). Further iterations of the sequence analysis identified additional variants of the motif at internal positions in all six proteins. Strikingly, nearly all internal occurrences of W[+]xxΨ were immediately followed by an aspartic acid (D) or a glutamic acid (E). Given that D and E both contain carboxyl groups, this finding suggested that a carboxyl group was important for the motif at that position, and the group was provided by either the carboxyl group at the C-terminus of the protein when the motif was found at the C-terminus, or by the D or E side chains when the motif was found internally (FIG. 12). These results suggested that the proteins shared a common motif, which had a consensus sequence of W[+]xxΨ[−] (SEQ ID NO: 28).

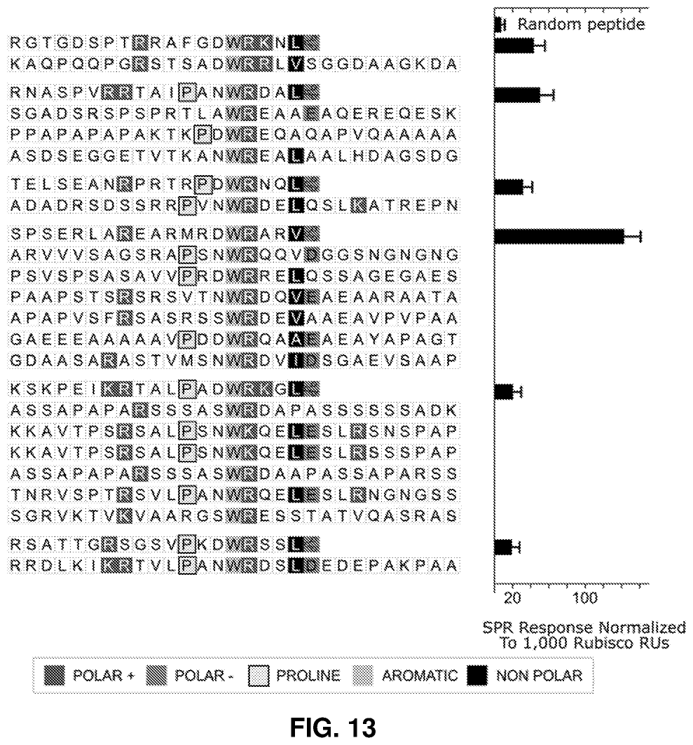

The W[+]xxΨ[−] Motif Binds to Rubisco Given that all six anti-RBM antibody interacting proteins also co-immunoprecipitated with Rubisco, and that the W[+]xxΨ motifs overlapped with RBMs in EPYC1, it was hypothesized that the W[+]xxΨ[−] motif bound to Rubisco. The results of the SPR assays showed that all of the synthetic peptides tested bound to Rubisco in vitro (FIG. 13).

The results presented in this example showed that multiple pyrenoid-associated proteins contained a W[+]xxΨ[−] motif that was recognized by an anti-RBM antibody, and that the W[+]xxΨ[−] motif bound to Rubisco.

### Example 5

### The W[+]xxΨ[−] Motif Targets Proteins to the Pyrenoid and Directs the Structural Organization of the Pyrenoid.

This example describes in vivo imaging experiments demonstrating that the W[+]xxΨ[−] RBM was sufficient to target proteins to the C. reinhardtii pyrenoid and that proteins containing the motif were localized to the pyrenoid.

### Materials and Methods

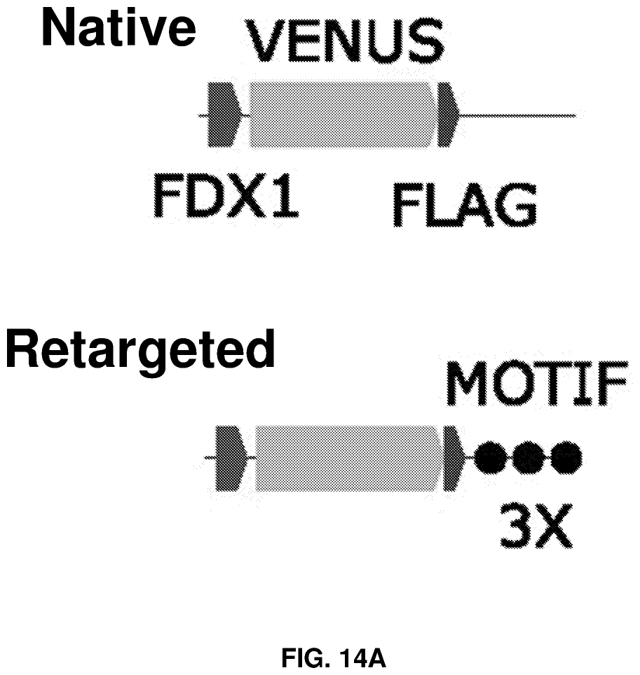

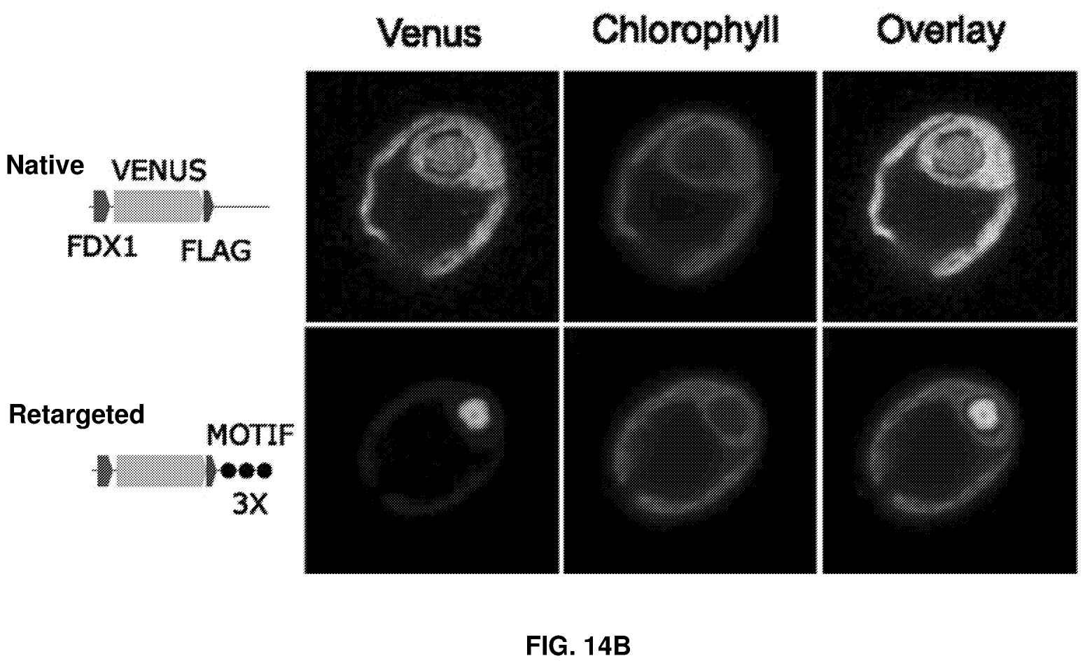

FDX1 Construct: As shown in FIG. 14A, the small highly abundant ferredoxin 1 protein (FDX1) was fused to the Venus fluorescent protein, three copies of the SAGA2 C-terminal 15 amino acids, and a FLAG tag. FDX1 natively localized throughout the chloroplast, including the pyrenoid matrix (FIG. 14B). A synthetic peptide (Invitrogen) containing a 643 bp restriction fragment containing the C-terminus of Venus, followed by the sequence coding for the FLAG-tag sequence, and a sequence coding for three repetitions of the 15 C-terminal amino acids of SAGA2, was cloned into pLM005-FDX1, after restriction digestion with EcoRl and PfIMI. GenBank accession number of the empty pLM005 is KX077945.1. The plasmid pLM005-FDX1 is identical to pLM005 with the genomic sequence of FDX1 cloned in frame by Gibson Assembly (Mackinder et al., PNAS 113: 5958-5963, 2015) between residues 2698 and 3234. The terminal 85 amino acids of resultant mature fusion protein, immediately downstream of the FLAG-tag, were a 5 aa linker (GGGGS; SEQ ID NO: 87), a first copy of SAGA2 15 C-terminal aa, followed by a 10 aa linker (2X GGGGS; SEQ ID NO: 88), a second copy of SAGA2 15 last aa, another 10 aa linker (2X GGGGS; SEQ ID NO: 88), and finally a third copy of SAGA2 15 last aa. The sequence of the EcoRI-PfIMI digestion fragment (SEQ ID NO: 89) was cloned in frame into pLM005-FDX1.

Culturing and Transformation of C. reinhardtii: Culturing and transformation of C. reinhardtii for fluorescence localization of protein and imaging was performed as described in Mackinder et al. (PNAS 113: 5958-5963, 2015).

Confocal Microscopy: Imaging was performed as described in Mackinder et al. (PNAS 113: 5958-5963, 2015), using a Leica SP5 equipped with high sensitivity hybrid detectors.

Electron Microscopy: QFDE microscopy was performed as described in Mackinder et al. (PNAS 113: 5958-5963, 2015).

### Results

The W[+]xxΨ[−] Motif is Sufficient to Target a Soluble Chloroplast Protein to the Pyrenoid: The interactions of the W[+]xxΨ[−] motif with Rubisco suggested that the motif mediated the localization of proteins containing the motif to the pyrenoid. The capacity of the motif to re-target FDX1, a ubiquitous chloroplast protein, to the pyrenoid was therefore determined by the fusion of FDX1 with three copies of the SAGA2 C-terminal 15 amino acids (“Retargeted”) (FIG. 14A).

As shown in FIG. 14B, FDX1 fused to the Venus fluorescent protein localized to throughout the chloroplast, including the pyrenoid matrix (“Native”). In contrast, “Retargeted” FDX1 fused to the Venus fluorescent protein localized almost exclusively to the pyrenoid (FIG. 14B).

The retargeting of the relatively small FDX1 fusion protein to the pyrenoid matrix did not violate the size exclusion principle that had been proposed, since the total size of the FDX1 fusion protein was approximately 43 kDa (<13 kDa FDX1, about 27 kDa fluorophore, about 3 kDa FLAG tag).

These results demonstrated that the W[+]xxΨ[−] motif was sufficient to recruit a protein to the pyrenoid.

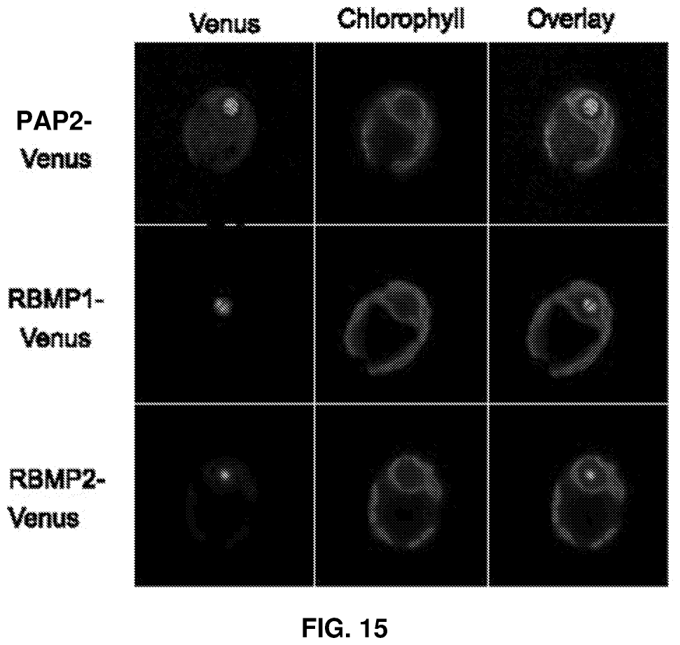

Four Previously-Uncharacterized Proteins with W[+]xxΨ[−] Motifs Localize to Regions of the Pyrenoid that Interact with the Matrix: The prediction from the pyrenoid proteome in FIG. 11A that the previously-uncharacterized Rubisco-binding proteins uncovered in Example 4 were bona fide pyrenoid-localized proteins was tested. Fluorescently-tagged PAP2-Venus, RBM P1-Venus and RBMP2-Venus all localized to the pyrenoid (FIG. 15). However, the fluorescence signals observed were quite distinct from the matrix-wide distribution of EPYC1.

PAP2 had a relatively uniform and continuous localization at the periphery of the Rubisco matrix surface but within the starch sheath. RBM P2 was confined to the very heart of the pyrenoid, a locus where tubules are known to intersect into a knot-like network. The observed localization pattern of PAP2 suggested that the protein acted as a bridge between the Rubisco matrix and the starch sheath.

The RBMP1 signal was more widespread than RBMP2 but distinctively limited to an inner sphere of the Rubisco matrix, and was bisected by a signal-less area. The observed localization patterns of RBMP1 and RBMP2 suggested that the proteins bridged the Rubisco matrix and intra-pyrenoidal photosynthetic membrane tubules.

These results suggested a simple model for the assembly of the pyrenoid structure (FIG. 16A) that centers around the binding of proteins to Rubisco via RBMs (FIG. 16B). Although proteins with RBMs likely compete for binding to the same site on Rubisco, the eight-fold symmetry of Rubisco allows for multiple and not necessarily competing interactions with multiple proteins. Thus, RBMs mediate interaction between Rubisco and EPYC1, as well as between the Rubisco matrix and other pyrenoid features, such as membrane tubules and starch sheaths.

### Example 6

### RBMs are Conserved Across Species

This example describes phylogenetic analyses that revealed RBMs were conserved across several algal species.

### Materials and Methods

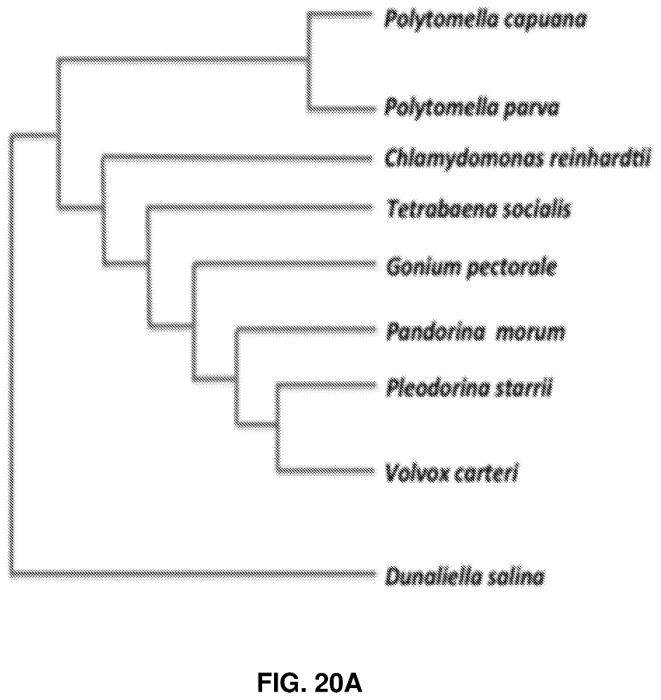

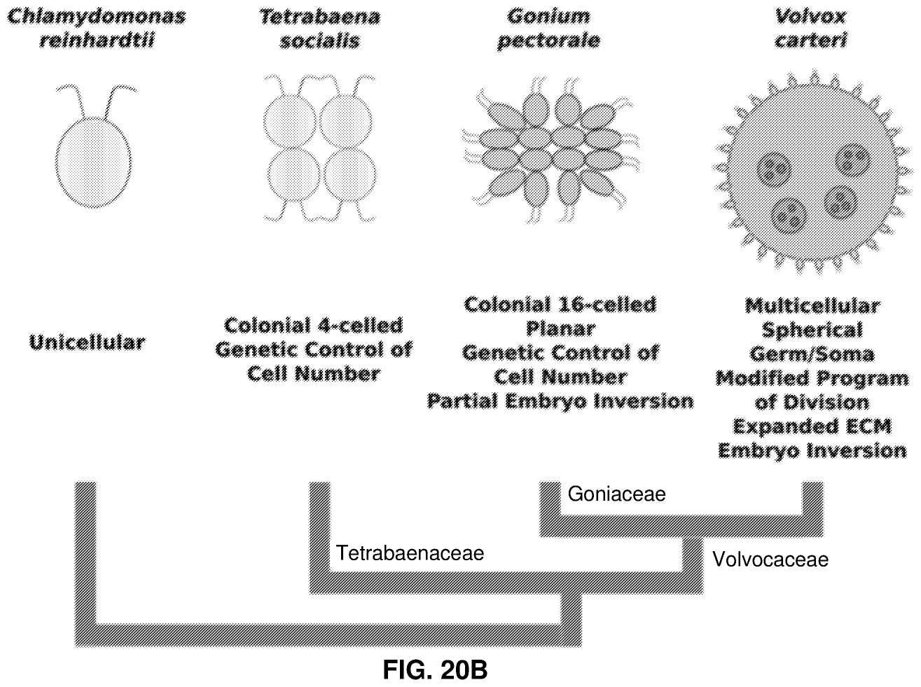

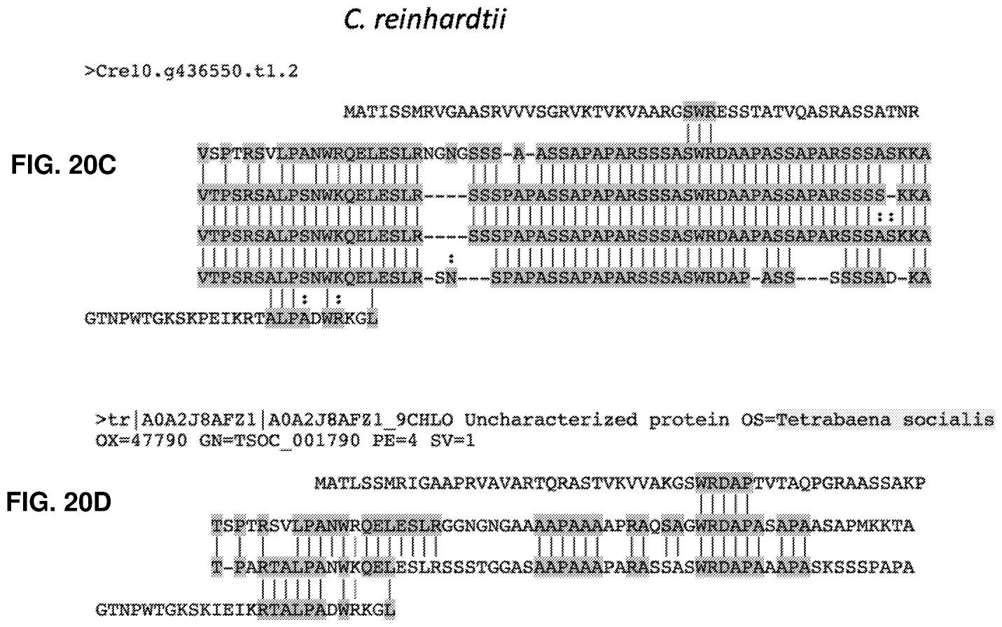

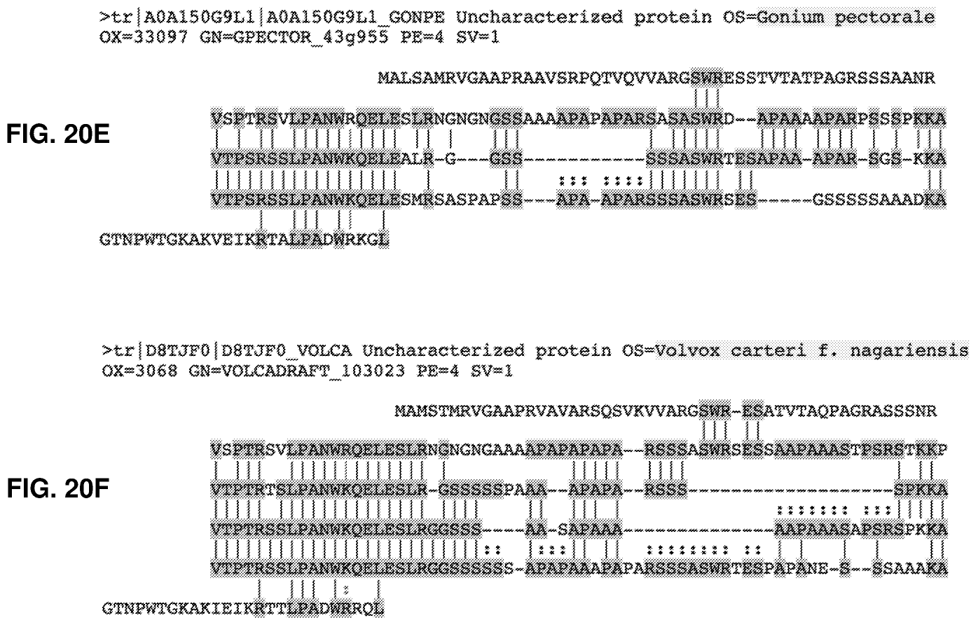

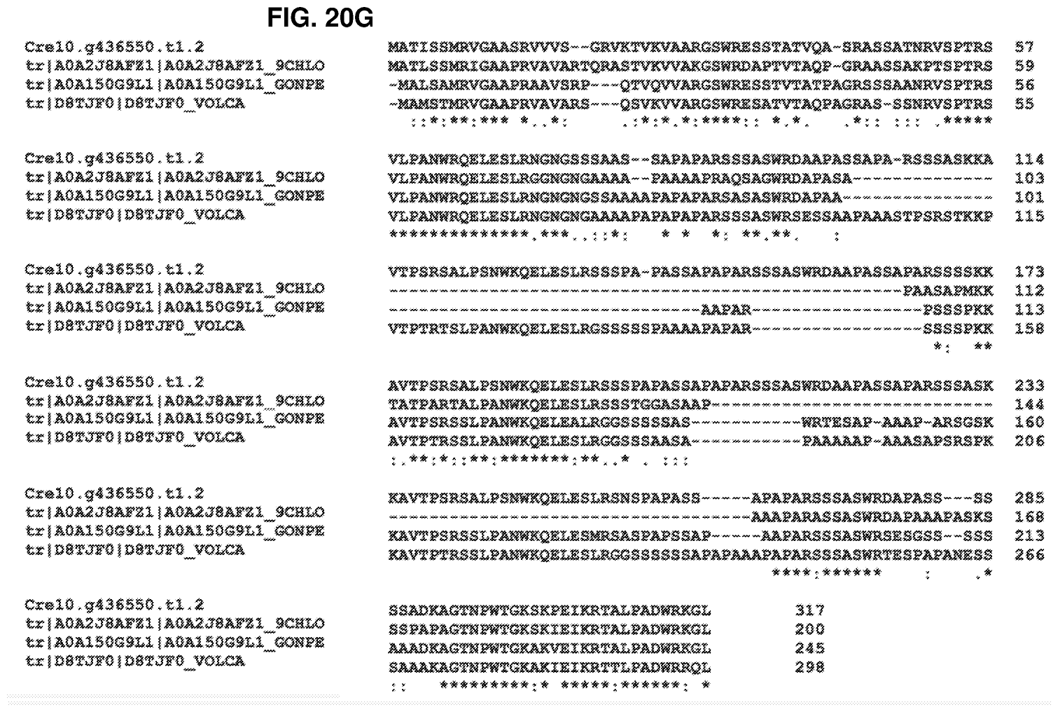

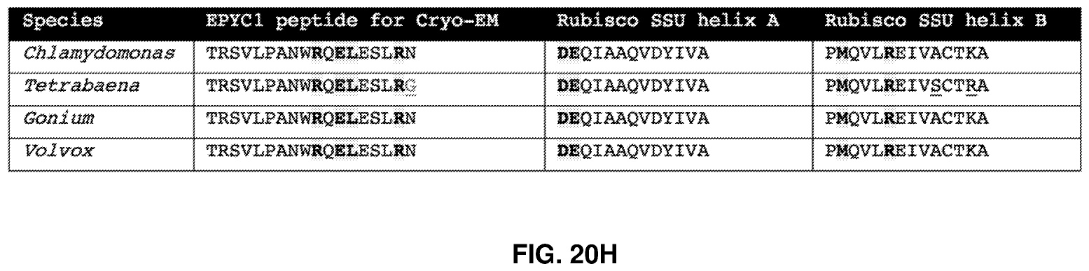

Phylogenetic Analysis: The sequences of EPYC1, EPYC1-like proteins, and Rubisco SSUs were analyzed in green algal species Chlamydomonas reinhardtii, Tetrabaena socialis, Gonium pectorale and Volvox carteri. FIG. 20A shows a phylogenetic tree of green algal species. FIG. 20B shows evolutionary trends during green algal evolution.

### Results

RBMs From EPYC1 Are Conserved Across Algal Species: An alignment of EPYC1 and EPYC1-like full length protein sequences from the four species revealed that the number of RBMs was not conserved between species. For example, C. reinhardtii EPYC1 had ten RBMs, whereas the EPYC1 or EPYC1-like proteins in T. socialis, G. pectorale, and V. carteri (FIGS. 20C-20F) had six, eight, and eight RBMs, respectively. This variation in the number of RBMs suggested that the exact number of binding sites may not be critical for function. This again supported the model that the formation of the Rubisco matrix primarily depends on multivalent interactions between EPYC1 and Rubisco (see Example 1).

As shown in FIG. 20H, comparison of the amino acid sequences of the helix region of EPYC1 RBM 2 showed that key residues were conserved among the four species. Moreover, alignment of the amino acid sequences of the α-helices of Rubisco SSU in the four species showed that key residues for binding to RBMs were conserved, including those residues that were identified as critical for binding to EPYC1 (compare with FIG. 9C). These results suggested that RBMs on EPYC1 and RBM-binding sites on Rubisco have co-evolved during algal evolution (FIG. 20B).

Alignment of the amino acid sequences of Rubisco SSUs from C. reinhardtii and Spinacia oleracea revealed that the key EPYC1-binding residues of the C. reinhardtii SSU were not conserved in S. oleracea. This result demonstrates that plant Rubisco SSUs do not contain the key EPYC1-binding residues required for interaction with EPYC1 RBMs.

### Example 7

### Addition of RBMs to Rubisco Induces EPYC1-Independent Rubisco Matrix Formation

This example describes representative methods for engineering Rubisco to form a Rubisco matrix independent of EPYC1. In addition, methods for determining whether an EPYC1-independent Rubisco matrix is formed by engineered Rubisco are provided.

### Materials and Methods

Fusion of Rubisco to RBMs: A Rubisco subunit protein is fused to one or more RBMs. RBMs are fused to either the small or large subunit of Rubisco. The RBM is appended to the RBM-binding site on Rubisco, such that it does not bind to any of that Rubisco holoenzyme's own RBM-binding sites.

Generation of Plants with Modified Rubisco SSU: The Rubisco SSU in plants, such as C3 plants, is modified to contain one or more RBM-binding sites, such as the RBM-binding sites or critical residues for binding to RBMs described in Example 2. In addition, the SSU is modified as described above to also include one or more RBMs. The RBMs and RBM-binding sites or critical residues for binding to RBMs in some embodiments are from the same algal species, e.g., C. reinhardtii.

Generation of Plants with a Rubisco SSU From Chlamydomonas reinhardtii: The Rubisco SSU in plants, such as C3 plants, is replaced with the Rubisco SSU from C. reinhardtii. In addition, the SSU is modified as described above to also include one or more RBMs. The RBMs and RBM-binding sites are from the same algal species, e.g., C. reinhardtii.

Pyrenoid-ready variants of the Rubisco SSU now exist in A. thaliana (Atkinson et al., New Phytol. 214: 655-667, 2017). These plants will be used as hosts to introduce Rubisco subunit proteins fused to one or more RBMs, using the same techniques and expression vectors that have been developed and tested previously (Atkinson et al., Plant Biotechnol. J. 14: 1302-1312, 2016).

### Results

Fusion of Rubisco to RBMs is Sufficient to Induce Rubisco Clustering and Matrix Formation in Chlamydomonas reinhardtii: As shown in the preceding Examples, RBMs interact with the Rubisco SSU of Chlamydomonas reinhardtii.

Thus, fusion of one or more RBMs to the Rubisco SSU will lead to clustering of Rubisco holoenzymes through the interaction between Rubisco SSU (either algal Rubisco SSU or modified Rubisco SSU) and the RBMs fused to Rubisco SSU. Similarly, fusion of one or more RBMs to the large subunit of Rubisco (LSU) will lead to clustering of Rubisco holoenzymes through the interaction between Rubisco SSU (either algal Rubisco SSU or modified Rubisco SSU) and the one or more RBMs fused to Rubisco LSU.

Clustering of Rubisco will lead to the formation of a Rubisco matrix in the chloroplast, independent of EPYC1.

In vitro phase separation experiments will show clustering of modified Rubisco in the absence of EPYC1.

In vivo imaging experiments using confocal fluorescence microscopy or electron microscopy will show clustering of modified Rubisco and formation of a Rubisco matrix in C. reinhardtii cells even when functional EPYC1 is not present.

Fusion of Rubisco to RBMs and Modification of Rubisco SSU are Sufficient to Induce Rubisco Clustering and Matrix Formation in Plants: To engineer Rubisco holoenzymes in plants to bind to RBMs, the Rubisco SSU in plant cells will be replaced with the SSU from C. reinhardtii. Consequently, assembled Rubisco holoenzymes will contain SSUs from C. reinhardtii, which, as shown in the preceding Examples, is capable of binding to RBMs. Further modification of Rubisco by the fusion of the LSU and/or SSU to one or more RBMs will lead to clustering of Rubisco holoenzymes through the interaction between the C. reinhardtii SSU and RBMs.

Alternatively, Rubisco holoenzymes in plants will be engineered to bind to RBMs by modifying the plant SSU with the addition of one or more RBM-binding sites. Consequently, assembled Rubisco holoenzymes will include SSUs that are capable of binding to RBMs. Further modification of Rubisco by fusion of the LSU and/or SSU to one or more RBM will lead to clustering of Rubisco holoenzymes through the interaction between modified SSUs and RBMs.

In vitro phase separation experiments will show clustering of modified Rubisco in the absence of EPYC1. Immunoprecipitation assays on non-denatured total protein extracts from the engineered plants described above will show clustering of modified Rubisco in the absence of EPYC1.

In vivo imaging experiments using confocal fluorescence microscopy or electron microscopy will show clustering of modified Rubisco and formation of a Rubisco matrix in plant cells even when functional EPYC1 is not present.

### Example 8

### Addition of RBMs to Proteins Promotes their Binding to Rubisco in Plants

This example describes representative methods for engineering proteins to bind to Rubisco. In addition, representative methods for determining whether an engineered protein binds Rubisco are provided.

### Materials and Methods

Fusion of Proteins to RBMs: A target protein is modified by addition of one or more RBMs. FDX1 is modified by addition of RBMs, as described in Example 5.

Generation of Plants with a Modified Rubisco SSU or a Rubisco SSU from C. reinhardtii and a Target Protein Fused to RBMs: The plants containing Modified Rubisco SSU or C. reinhardtii Rubisco SSU (generated in Example 7) are engineered to also contain target protein fused to RBMs. The plants containing Modified Rubisco SSU or C. reinhardtii Rubisco SSU (generated in Example 7) are engineered to also contain FDX1 fused to RBMs. In some embodiments, the RBMs are from the same algal species as the algal Rubisco SSU or the RBM-binding sites or critical residues for binding to RBMs of the modified Rubisco SSU, e.g., C. reinhardtii.

### Results

Recruitment of Proteins to Rubisco and to the Pyrenoid in Plants: A target protein will be modified by the addition of one or more RBMs.

Plant Rubisco will be modified by replacing the plant Rubisco SSU with the C. reinhardtii SSU. Alternatively, the plant Rubisco SSU will be modified by addition of one or more RBM-binding sites.

In vitro co-immunoprecipitation will show that the modified target protein binds to the modified plant Rubisco through the interaction between the one or more RBMs and modified Rubisco SSU.

In vivo co-immunoprecipitation experiments from plant cell lysates will show that the modified target protein binds and co-immunoprecipitates with modified plant Rubisco.

In vivo imaging experiments using confocal microscopy or electron microscopy will show that the modified target protein co-localizes with modified plant Rubisco.

In addition, in vivo imaging experiments using confocal microscopy or electron microscopy will show that the modified target protein localizes to the Rubisco matrix in pyrenoids through its interaction with modified plant Rubisco.

Further, in vivo imaging experiments using confocal microscopy or electron microscopy will show that the modified FDX1 localizes to the Rubisco matrix in the pyrenoid through its interaction with Rubisco.

### Example 9

### Addition of RBMs to Proteins Promotes their Recruitment to Specific Regions of the Pyrenoid

This example describes representative methods for engineering proteins to be recruited to specific regions of the pyrenoid. In addition, methods for determining the localization of engineered proteins are provided.

### Materials and Methods

Fusion of Proteins to RBMs: A soluble target protein is modified by the addition of one or more RBMs. Plant cells are transformed with a construct encoding the modified target protein. Cloning green algal genes into a higher plant expression vector, and optimizing chloroplast targeting, is done as previously described (Atkinson et al., Plant Biotech. J. 14: 1302-1312, 2016).

A target protein containing a starch-binding domain or a binding domain for a protein that binds starch is modified by the addition of one or more RBMs. The starch binding domain or the binding domain for a protein that binds starch can be native to the target protein or is fused to the target protein.

A target protein containing a membrane-associated domain (e.g., a thylakoid membrane-associated domain or a membrane tubule-associated domain) or a membrane protein binding domain (e.g., a thylakoid membrane protein binding domain or a membrane tubule protein binding domain) is modified by the addition of one or more RBMs. The RBMs are added to the target protein in a location that exposes the RBMs to the external surface of the membrane. The membrane-associated or membrane protein binding domain can be native to the target protein or will be fused to the target protein. The membrane associated protein is an algal RBMP. The membrane associated protein is C. reinhardtii RBMP1 or RBMP2.

Generation of Plants with a Modified Rubisco SSU or a Rubisco SSU from C. reinhardtii and a Target Protein Fused to RBMs: The plants containing Modified Rubisco SSU or C. reinhardtii Rubisco SSU (generated in Example 7) are engineered to also contain a target protein containing a starch-binding domain fused to RBMs. The plants containing Modified Rubisco SSU or C. reinhardtii Rubisco SSU (generated in Example 7) are engineered to also contain a target protein containing a membrane-associated domain fused to RBMs. The plants containing Modified Rubisco SSU or C. reinhardtii Rubisco SSU (generated in Example 7) are engineered to also contain RBMPs fused to RBMs. In representative embodiments, the RBMs are from the same algal species as the algal Rubisco SSU or the RBM-binding sites or critical residues for binding to RBMs of the modified Rubisco SSU, e.g., C. reinhardtii.

### Results

Recruitment of Proteins to the Rubisco Matrix in the Pyrenoid in Plants: In vivo imaging experiments using confocal microscopy or electron microscopy will show that a soluble target protein modified by the addition of one or more RBMs localizes to the Rubisco matrix in pyrenoids through its interaction with the Rubisco SSU from C. reinhardtii or a plant SSU modified by addition of one or more RBM-binding sites or critical residues for binding to RBMs.

Recruitment of Proteins to Rubisco Matrix-Starch Sheath Interface in the Pyrenoid Plants: In vivo imaging experiments using confocal microscopy or electron microscopy will show that a modified target protein (containing a starch-binding domain or a binding domain for a protein that binds starch) that is modified by the addition of one or more RBMs localizes to the Rubisco matrix-starch sheath interface in pyrenoids through its interaction with modified plant Rubisco.

A target protein may have one or more activities that will be localized to the Rubisco matrix-starch sheath interface using the methods described in this example.

Recruitment of Proteins to Rubisco Matrix-Membrane Interface in the Pyrenoid in Plants: In vivo imaging experiments using confocal microscopy or electron microscopy will show that the a modified target protein containing a membrane-associated domain (e.g., a thylakoid membrane-associated domain or a membrane tubule-associated domain) or a membrane protein binding domain (e.g., a thylakoid membrane protein binding domain or a membrane tubule protein binding domain) modified by the addition of one or more RBMs localizes to the Rubisco matrix-membrane interface in pyrenoids through its interaction with modified plant Rubisco and association with the membrane.

A target protein may have one or more activities that will be localized to the Rubisco matrix-membrane interface using the methods described in this example.

As will be understood by one of ordinary skill in the art, each embodiment disclosed herein can include, consist essentially of or consist of its particular stated element, step, ingredient or component. Thus, the terms “include” or “including” should be interpreted to recite: “include, consist of, or consist essentially of.” The transition term “include” or “includes” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect, in this context, is a measurable change in binding between two proteins or a protein and a peptide, or a measurable change in the CO₂ fixation rate or efficiency of a plant or plant cell.

Unless otherwise indicated, all numbers expressing quantities of ingredients, properties such as molecular weight, reaction conditions, and so forth used in the specification and embodiments are to be understood as being modified in all instances by the term “about.” Accordingly, unless indicated to the contrary, the numerical parameters set forth in the specification and attached embodiments are approximations that may vary depending upon the desired properties sought to be obtained by the present invention. At the very least, and not as an attempt to limit the application of the doctrine of equivalents to the scope of the embodiments, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following embodiments) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise embodimented. No language in the specification should be construed as indicating any non-embodimented element essential to the practice of the invention.

Groupings of alternative elements or embodiments of the invention disclosed herein are not to be construed as limitations. Each group member may be referred to and embodimented individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended embodiments.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the embodiments appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, sequence database entries (current as of Aug. 2, 2019), and other written text throughout this specification (referenced materials herein). Each of the referenced materials is individually incorporated herein by reference in its entirety for its referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the figures/drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. (canceled)

2. A genetically altered higher plant or part thereof, comprising:
a chimeric polypeptide comprising one or more Rubisco-binding motifs (RBMs) and a heterologous polypeptide; or
a stabilized polypeptide comprising two or more RBMs and a Rubisco SSU protein, wherein the Rubisco SSU protein is an algal Rubisco SSU protein or a modified higher plant Rubisco SSU protein that comprises one or more amino acid substitutions for an algal Rubisco SSU corresponding to residues 23, 24, 87, 90, 91, and 94 in SEQ ID NO: 60.

3. The plant or part thereof of claim 2, wherein the one or more RBMs are independently selected from the group consisting of polypeptides having at least 80% sequence identity, at least 85% sequence identity, at least 90% sequence identity, at least 95% sequence identity, at least 96% sequence identity, at least 97% sequence identity, at least 98% sequence identity, or at least 99% sequence identity to at least one of SEQ ID NO: 27 or SEQ ID NO: 28.

4. The plant or part thereof of claim 2, wherein the heterologous polypeptide comprises a Rubisco Small Subunit (SSU), a Rubisco Large Subunit (LSU), a 2-carboxy-d-arabinitol-1-phosphatase (CA1P), a xylulose-1,5-bisphosphate (XuBP), a Rubisco activase, a protease-resistant non-EPYC1 linker, a membrane anchor, or a starch binding protein.

5. The plant or part thereof of claim 4, wherein the heterologous polypeptide is the Rubisco SSU and the one or more RBMs are linked to the N-terminus or C-terminus of the Rubisco SSU, optionally through a linker polypeptide.

6. The plant or part thereof of claim 2, wherein the plant or part thereof further comprises an algal Rubisco SSU protein or a modified higher plant Rubisco SSU protein.

7. The plant or part thereof of claim 6, wherein the Rubisco SSU protein is the algal Rubisco SSU protein, and wherein the one or more RBMs and the algal Rubisco SSU protein are from the same algal species.

8. The plant or part thereof of claim 6, wherein the Rubisco SSU protein is the modified higher plant Rubisco SSU protein, and wherein the modified higher plant Rubisco SSU comprises one or more amino acid substitutions for an algal Rubisco SSU corresponding to residues 23, 24, 87, 90, 91, and 94 in SEQ ID NO: 60.

9. The plant or part thereof of claim 8, wherein:
the amino acid substitution is at residue 23 and the substituted amino acid is Glu or Asp;
the amino acid substitution is at residue 24 and the substituted amino acid is Glu or Asp;
the amino acid substitution is at residue 87 and the substituted amino acid is Ala, Ile, Leu, Met, Phe, Trp, Tyr, or Val;
the amino acid substitution is at residue 90 and the substituted amino acid is Ala, Ile, Leu, Met, Phe, Trp, Tyr, or Val;
the amino acid substitution is at residue 91 and the substituted amino acid is Arg, His, or Lys; and/or
the amino acid substitution is at residue 94 and the substituted amino acid is Ala, Ile, Leu, Met, Phe, Trp, Tyr, or Val.

10. The plant or part thereof of claim 4, wherein the heterologous polypeptide is the Rubisco LSU and the one or more RBMs are linked to the N-terminus or C-terminus of the Rubisco LSU, optionally through a linker polypeptide.

11. The plant or part thereof of claim 4, wherein the heterologous polypeptide is the membrane anchor and the membrane anchor anchors the heterologous polypeptide to a thylakoid membrane of a chloroplast and is optionally selected from the group consisting of a membrane bound protein, a protein that binds to a membrane-bound protein, a transmembrane domain, and a lipidated amino acid residue in the heterologous polypeptide.

12. The plant or part thereof of claim 4, wherein the heterologous polypeptide is the starch binding protein and the starch binding protein comprises an alpha-amylase/glycogenase;
a cyclomaltodextrin glucanotransferase; a protein phosphatase 2C 26; an alpha-1,4-glucanotransferase; a phosphoglucan, water dikinase; a glucan 1,4-alpha-glucosidase; or a LCI9.

13. The plant or part thereof of claim 2, wherein the chimeric polypeptide is localized to a chloroplast stroma of at least one chloroplast of a plant cell of the plant or part thereof, and wherein the plant cell is a photosynthetic cell.

14. The plant or part thereof of claim 2, wherein the plant is a C3 crop plant selected from the group consisting of cowpea, soybean, cassava, rice, wheat, plantain, yam, sweet potato, and potato.

15. A genetically altered higher plant or part thereof, comprising:
(A) a polypeptide comprising two or more RBMs, and one or both of:
an algal Rubisco-binding membrane protein (RBMP); and
a Rubisco SSU protein; or
(B) an algal Rubisco SSU protein, and
at least one of the following:
a stabilized polypeptide comprising two or more RBMs;
a polypeptide containing part or all of an algal Rubisco-binding membrane protein (RBMP); or
one or more RBMs fused to a heterologous polypeptide that localizes to a thylakoid membrane of a chloroplast, wherein the heterologous polypeptide that localizes to a thylakoid membrane of a chloroplast comprises at least one of:
a membrane bound protein,
a protein that binds to a membrane-bound protein,
a transmembrane domain, or
a lipidated amino acid residue in the heterologous polypeptide.

16. The plant or part thereof of claim 15, wherein the polypeptide is a stabilized polypeptide that has been modified to remove one or more chloroplastic protease cleavage sites, and wherein the polypeptide optionally comprises EPYC1 or CSP41A.

17. A method of producing the genetically altered plant of claim 2, comprising:
a) introducing a first nucleic acid sequence encoding the chimeric polypeptide comprising one or more RBMs and the heterologous polypeptide or the polypeptide comprising two or more RBMs, and optionally introducing a second nucleic acid sequence encoding the Rubisco SSU protein into a plant cell, tissue, or other explant;
b) regenerating the plant cell, tissue, or other explant into a genetically altered plantlet; and
c) growing the genetically altered plantlet into a genetically altered plant comprising the first nucleic acid sequence encoding the chimeric polypeptide comprising one or more RBMs and the heterologous polypeptide, and optionally, the second nucleic acid sequence.

18. A method of producing the genetically altered plant of claim 15, comprising:
a) introducing a first nucleic acid sequence encoding a stabilized polypeptide comprising two or more RBMs, and introducing one or both of a second nucleic acid sequence encoding the algal RBMP and a third nucleic acid sequence encoding the Rubisco SSU protein into a plant cell, tissue, or other explant;
b) regenerating the plant cell, tissue, or other explant into a genetically altered plantlet; and
c) growing the genetically altered plantlet into a genetically altered plant comprising the first nucleic acid sequence encoding the stabilized polypeptide comprising two or more RBMs, and one or both of the second nucleic acid sequence encoding the algal Rubisco-binding membrane protein (RBMP) and the third nucleic acid sequence encoding the Rubisco SSU protein.

19. A chimeric polypeptide comprising one or more, two or more, or three or more Rubisco-binding motifs (RBMs) and a heterologous polypeptide, wherein the RBM comprises the peptide sequence W[+]xxΨ[−] (SEQ ID NO: 28), SEQ ID NO: 27, or an amino acid sequence motif comprising WR or WK, where the W is assigned to position ‘0’, and which motif scores 5 or higher using the following criteria:
points are assigned as follows:
R or K in −6 to −8: +1 point;
P in −3 or −2: +1 point;
D/N at −1: +1 point;
optionally D/E at +2 or +3: +1 point;
A/I/L/V at +4: +2 points; and
D/E/COO— terminus at +5: +1 point.

20. (canceled)

21. (canceled)
